(19) United States
(12) Patent Application Publication
(10) Pub. No.: US 2023/0358740 A1
(43) Pub. Date: Nov. 9, 2023

(54) METHODS FOR DETECTION OF MICROBES AND MICROBE COMPONENTS

(71) Applicant: PRESIDENT AND FELLOWS OF HARVARD COLLEGE, Cambridge, MA (US)

(72) Inventors: Mark Joseph CARTWRIGHT, Newton, MA (US); Michael SUPER, Lexington, MA (US); Donald E. INGBER, Boston, MA (US); Jennifer GRANT, Cambridge, MA (US); Justin SCOTT, Cambridge, MA (US); Shannon Catherine DUFFY, Cambridge, MA (US); Sahil LOOMBA, Cambridge, MA (US)

(73) Assignee: PRESIDENT AND FELLOWS OF HARVARD COLLEGE, Cambridge, MA (US)

(21) Appl. No.: 17/791,609

(22) PCT Filed: Jan. 8, 2021
(86) PCT No.: PCT/US2021/012717

§ 371 Date: Jul. 8, 2022

Related U.S. Application Data
(60) Provisional application No. 62/959,286, filed on Jan. 10, 2020.

(51) Int. Cl. G01N 33/569; G01N 33/68

(52) U.S. Cl. CPC G01N 33/56911; G01N 33/6851

(57) ABSTRACT

The technology described herein is directed to methods for detection of microbes and microbe components. In some embodiments of any of the aspects, the methods comprise methods of microbe isolation, sample preparation, mass spectrometry, or analysis. In some embodiments of any of the aspects, such methods can be applied to detect at least one microbe or at least one microbial component in a sample, including not limited to a patient sample, an animal model sample, an environmental sample, or a non-biological sample.

### CROSS-REFERENCE TO RELATED APPLICATIONS

This application claims benefit under 35 U.S.C. § 119(e) of U.S. Provisional Application No. 62/959,286 filed Jan. 10, 2020, the contents of which are incorporated herein by reference in their entirety.

### GOVERNMENT SUPPORT

This invention was made with government support under Grant Nos. W911NF-16-C-0050 and W911NF1920027 awarded by the Defense Advanced Research Projects Agency. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jan. 8, 2021, is named 002806-095320WOPT_SL.txt and is 29,480 bytes in size.

### TECHNICAL FIELD

The technology described herein relates to methods for detection of microbes and microbe components.

### BACKGROUND

The detection of microbes, whether in a biological or non-biological sample, is a fundamental need that spans the fields of medicine and research. In time-sensitive situations such as a septic infection, it is critical that such methods detect microbes in a short period of time. A matter of minutes can mean life or death in a patient.

For example, septic infections occur when pathogens or pathogen-derived toxins enter the circulation and trigger a rapid systemic inflammatory response. There are over 30 million cases of sepsis associated with over 5 million deaths every year. Septic patients require fast-acting treatment and are typically initially cared for with empirical, broad-spectrum antibiotics until an identification can be made. Broad-spectrum antibiotics are associated with higher toxicity, increased healthcare costs, and may not be optimal for treatment of the infectious agent. Moreover, the patient probability of survival can decrease by 7 to 12% for every hour in which an appropriate antibiotic is not administered. Alternatively, patient outcomes and patient management can be improved by pathogen identification and subsequent application of targeted therapies.

The implementation of Matrix-Assisted Laser Desorption-Ionization Time-of-Flight mass spectrometry (MALDI-TOF MS) in the analysis of positive cultures from infected patients has emerged over recent years as a highly effective method for identifying microbial species. Commercially available MALDI-TOF MS systems such as the Biotyper from Bruker Daltonics Inc. or the VITEK MS from bioMérieux are in use in clinical laboratories for identification of species from culture positive septic patients (see e.g., Biswas and Rolain, 2013, J of Micro Methods 92, 14-24; Cherkaoui et al., 2010, J Clin Microbiol. 48(4), 1169-1175; Croxatto et al., 2011, FEMS Microbiol Rev. 36 (2), 380-407; Martin, 2017, Mass Spectrometry for the Clinical Laboratory. 11, 231-245; Saffert et al, 2011. J Clin Microbiol. 49 (3), 887-892; Singhal et al, 2015. Front Microbiol. 6:791; each of which is incorporated by reference herein in its entirety).

These systems can provide a rapid and accurate method of identification in a subset of cases, however they have several limitations that severely restrict their applicability as a sepsis diagnostic. Most notably, a patient must be blood culture positive, meaning clinicians must be able to extract and cultivate at least 1×10⁵ colony-forming units per milliliter (CFU/mL) of live pathogen from patient blood (see e.g., Christner et al., 2010, J Clin Microbiol. 48 (5), 1585-1591; Biswas and Rolain, supra; Cherkaoui et al., 2010, supra; Martin, 2017, supra; Cartwright et al., 2016, EBioMedicine 9, 217-227; Ho and Reddy, 2010, Clin Chem. 56(4), 525-536; Idelevich et al, 2014, Clin Micro and Infect. 20 (10), 1001-1006; each of which is incorporated by reference herein in its entirety).

However, only 15-30% of septic patients are blood culture positive (see e.g., Cartwright et al., 2016, supra; Gille-Johnson et al., 2013, Scand. J. Infect. Dis. 45 (3), 186-193; Tsalik et al., 2012, J. Emerg. Med. 43 (1), 97-106; each of which is incorporated by reference herein in its entirety). Most patients have only <1 to 100 CFU/mL of live pathogen present in their blood during infection (see e.g., Li et al., 2017, SLAS Technol. 22(6): 545-608, which is incorporated by reference herein in its entirety). Moreover, current standard-of-care with simultaneous antibiotic treatment may render bacteria nonviable; leading the majority of septic patients to yield blood culture negative results (see e.g., Li et al., 2017, supra; Bacconi et al., 2014, J. Clin. Microbiol. 52(9), 3164-3174; Kang et al., 2014, Nat. Med. 20 (10), 1211-1216; Prost et al., 2013, Crit Care. 17(5) 1001; each of which is incorporated by reference herein in its entirety). The applicability of these systems is further constrained by the 1-5 days required for a positive blood culture to appear. Although molecular methods, such as polymerase chain reaction (PCR), negate this cultivation requirement, the case applicability remains very similar to culture-based methods due to a lack of microbial DNA presence in the blood.

Furthermore, MALDI-TOF MS methods using cultured samples are still often unable to distinguish closely related species, such as Shigella spp. and E. coli (see e.g., van den Beld et al, 2019, Matrix-Assisted Laser Desorption-Ionization Time-of-Flight mass spectrometry using a custom-made database, biomarker assignment or mathematical classifiers does not differentiate Shigella spp. and Escherichia coli; accessible on the world wide web at biorxiv.org/content/10.1101/714295v1). There is a vital need for a technique capable of rapidly identifying the pathogen from the large majority of septic patients who are blood culture negative. This information would act as a highly effective guide for physicians to provide targeted, more effective therapy and reduce patient mortality.

### SUMMARY

The technology described herein is directed to methods for detection of microbes and microbe components. Described herein are methods of detecting at least one microbe or at least one microbe component, the method comprising the following steps: (i) contacting a sample with at least one engineered microbe-targeting molecule linked to a support; (ii) isolating the microbe(s) or microbe components bound to the at least one engineered microbe-targeting molecule; (iii) contacting the microbe(s) or microbe components with a matrix and/or matrix solution on a target substrate; and (iv) detecting the microbe(s) or microbe components using a mass spectrometric method.

Of note, the methods described herein comprise a step of isolating with an engineered microbe targeting molecule, which can eliminate the need for a culturing step. Accordingly, in some embodiments of any of the aspects, the methods described herein do not comprise a culturing step, e.g., a step involving culturing and/or maintaining the microbe(s) ex vivo or in vitro. Furthermore, the methods described herein can comprise a detection step, wherein the signal to noise ratio of data points obtained from a mass spectrometric method can be increased. In some embodiments of any of the aspects, the detection step comprises a clustering process and/or at least one weighting parameter, as described further herein.

Also described herein are methods of producing a profile for a microbe, e.g., for the identification of at least one microbe or at least one microbe component in a sample. In some embodiments of any of the aspects, the results of the detection step comprise a profile, wherein said profile indicates the presence or absence of at least one microbe or at least one microbe component. In some embodiments of any of the aspects, the profile is unique to a specific microbe or microbe component and as such can be used to identify an unknown microbe in sample.

According in one aspect, described herein is a method of detecting a microbe or microbe component, the method comprising the following steps: (i) contacting a sample with an engineered microbe-targeting molecule linked to a support; (ii) isolating the microbe or microbe components bound to the engineered microbe-targeting molecule; (iii) contacting the microbe or microbe components with a matrix or matrix solution on a target substrate; and (iv) detecting the microbe or microbe components using a mass spectrometric method.

In another aspect, described herein is a method of detecting a microbial infection in a patient, the method comprising the following steps: (i) contacting a patient sample with an engineered microbe-targeting molecule linked to a support; (ii) isolating the microbe or microbe components bound to the engineered microbe-targeting molecule; (iii) contacting the microbe or microbe components with a matrix or matrix solution on a target substrate; and (iv) detecting the microbe or microbe components using a mass spectrometric method.

In some embodiments of any of the aspects, the microbe components comprise microbe-associated molecular patterns (MAMPs).

In some embodiments of any of the aspects, the microbe components comprise pathogen-associated molecular patterns (PAMPs).

In some embodiments of any of the aspects, the detecting of step iv outputs mass spectrometric data obtained from the sample as a sample library.

In some embodiments of any of the aspects, the detecting of step iv comprises comparing the sample library with at least one control library of mass spectrometric data.

In some embodiments of any of the aspects, the at least one control library of mass spectrometric data comprises data obtained from at least one control sample not comprising any known microbes or microbe components.

In some embodiments of any of the aspects, the detecting of step iv comprises comparing the sample library with at least one reference library of mass spectrometric data.

In some embodiments of any of the aspects, the at least one reference library of mass spectrometric data comprises data obtained from at least one sample comprising a known microbe or components of at least one known microbe.

In some embodiments of any of the aspects, the detecting of step iv comprises analyzing the sample library with a control system comprising one or more processors, the control system configured to execute machine executable code using a clustering process, wherein each cluster comprises a cluster of data points from a single molecular signal of interest.

In some embodiments of any of the aspects, each cluster of data points is at least 1 Dalton (Da) wide.

In some embodiments of any of the aspects, the cluster of data points is based on m/z peaks identified by the maximum intensity of that cluster.

In some embodiments of any of the aspects, the cluster of data points is based on m/z peaks identified by the mean m/z value of that cluster.

In some embodiments of any of the aspects, the detection process further comprises removing at least one data point of the sample library or the control library, wherein the at least one data point comprises a repeatability value at or below a pre-determined threshold.

In some embodiments of any of the aspects, the clustering process further comprises removing at least one data point of the sample library that matches a data point in a control library within +/−0.3 Da.

In some embodiments of any of the aspects, the clustering process further comprises removing at least one data point of the sample library that does not match a data point in at least one reference library within +/−0.3 Da.

In some embodiments of any of the aspects, the detecting of step iv comprises determining a peak area ratio of at least one pair of data points within the sample library and at least one pair of data points in at least one reference library.

In some embodiments of any of the aspects, at least one peak area ratio of the sample library is compared to at least one corresponding peak area ratio of at least one reference library.

In some embodiments of any of the aspects, a score is calculated based on the comparison of at least one peak area ratio of the sample library and at least one corresponding peak area ratio of at least one reference library.

In some embodiments of any of the aspects, the clustering process further comprises applying a weighting parameter, comprising a frequency weighting parameter and/or a trustworthiness weighting parameter, wherein the weighting parameter identifies the proportion of data points that are unique to the sample library or common with a reference library or a control library.

In some embodiments of any of the aspects, the frequency weighting parameter increases the weight of a data point if the cluster containing the data point comprises additional other data points.

In some embodiments of any of the aspects, the trustworthiness weighting parameter decreases the weight of a data point if the data point is found within multiple clusters in the sample library, reference library, and/or control library.

In some embodiments of any of the aspects, the method further comprises: (a) assigning a score to the sample based on similarity with a reference library; and/or (b) identifying the microbe in the sample as belonging to a reference library based on the score being above a predetermined threshold.

In some embodiments of any of the aspects, the method further comprises identifying the species of the microbe detected in the sample according to the data points analyzed and outputting said species on a display.

In some embodiments of any of the aspects, the method further comprises identifying the strain of the microbe detected in the sample according to the data points analyzed and outputting said strain on a display.

In some embodiments of any of the aspects, the method further comprises determining whether the microbe detected in the sample is sensitive or resistant to an antimicrobial therapeutic according to the data points analyzed and outputting said sensitivity on a display.

In some embodiments of any of the aspects, the method further comprises assigning the patient to an infection category according to the data points analyzed and outputting the infection category on a display.

In some embodiments of any of the aspects, the results of step iv comprise a profile, wherein said profile indicates the presence or absence of at least one microbe or microbe component.

In some embodiments of any of the aspects, the profile is specific to at least one microbe or microbe component.

In some embodiments of any of the aspects, the profile comprises a set of data points for a specific microbe or specific set of microbes, and wherein each profile comprises a set of m/z peaks clustered for a single molecular signal of interest.

In some embodiments of any of the aspects, the profile for the specific microbe does not include any of the set of data points associated with a control library.

In some embodiments of any of the aspects, the profile is distinguishable from the profiles of other microbes or microbe components or sets thereof.

In some embodiments of any of the aspects, the profile is set forth in any one of FIG. 4A-4B, FIG. 5A-5G, FIG. 8B-8J, or FIG. 9A-9B.

In some embodiments of any of the aspects, the support is a magnetic support.

In some embodiments of any of the aspects, the support is a non-magnetic support.

In some embodiments of any of the aspects, the support is a non-magnetic nanoparticle.

In some embodiments of any of the aspects, the support is a mesoporous nanoparticle.

In some embodiments of any of the aspects, the support is mesoporous silica.

In some embodiments of any of the aspects, the step of isolating comprises applying a magnet to the sample.

In some embodiments of any of the aspects, the step of isolating comprises washing the support with a buffer to remove unbound cells or biomolecules.

In some embodiments of any of the aspects, the step of isolating further comprises eluting the microbe or microbe components from the support.

In some embodiments of any of the aspects, the step of eluting comprises heating to a temperature of at least 70° C. and/or shaking at a speed of at least 950 rpm for no longer than 30 minutes.

In some embodiments of any of the aspects, the heating to a temperature of at least 70° C. is performed in calcium-free water.

In some embodiments of any of the aspects, the step of eluting comprises treatment with ethylenediaminetetraacetic acid (EDTA).

In some embodiments of any of the aspects, the step of isolating does not comprise eluting the microbe or microbe components from the support.

In some embodiments of any of the aspects, the step of isolating comprises concentrating the microbe or microbe components into a smaller volume from a larger volume of the sample.

In some embodiments of any of the aspects, the isolated volume is less than the volume of the sample.

In some embodiments of any of the aspects, the target substrate is evenly sprayed with matrix solution prior to step iii to generate a homogenous layer of crystallized matrix on top of the target substrate.

In some embodiments of any of the aspects, the matrix solution comprises a matrix selected from the group consisting of 2′,6′-dihydroxyacetophenone (DHAP), α-Cyano-4-hydroxycinnamic acid (CHCA), sinapic acid (SA), super DHB, 2′,4′,6′-trihydroxyacetophenone monohydrate (THAP), and 9-aminoacridine (9-AA), and the matrix is dissolved in an organic, aqueous solution.

In some embodiments of any of the aspects, the matrix solution is 40 mg/mL 2,5-Dihydroxybenzoic acid (DHB) in 50% methanol, 50% water, 0.1% formic acid.

In some embodiments of any of the aspects, the mass spectrometric method is Matrix-Assisted Laser Desorption Ionization (MALDI-TOF).

In some embodiments of any of the aspects, the mass spectrometric method is automated.

In some embodiments of any of the aspects, the sample comprises blood, serum, plasma, sputum, urine, joint fluid, or any other tissue or biological sample.

In some embodiments of any of the aspects, the patient has been treated with antibiotics.

In some embodiments of any of the aspects, the sample contains at least one antibiotic.

In some embodiments of any of the aspects, the sample contains at least two antibiotics.

In some embodiments of any of the aspects, the patient has been treated with antifungals.

In some embodiments of any of the aspects, the sample contains antifungals.

In some embodiments of any of the aspects, the patient has been treated with antivirals.

In some embodiments of any of the aspects, the sample contains antivirals.

In some embodiments of any of the aspects, the sample has not been cultured.

In some embodiments of any of the aspects, the time from the step of collecting the sample to the end of detecting takes equal to or less than 90 minutes.

In some embodiments of any of the aspects, the engineered microbe-targeting molecule comprises a microbe surface-binding domain.

In some embodiments of any of the aspects, the microbe surface-binding domain comprises a mannose-binding lectin (MBL).

In some embodiments of any of the aspects, the microbe surface-binding domain comprises a human mannose-binding lectin (MBL).

In some embodiments of any of the aspects, the microbe surface-binding domain comprises a carbohydrate recognition domain (CRD) of MBL.

In some embodiments of any of the aspects, the CRD is linked to an immunoglobulin or fragment thereof.

In some embodiments of any of the aspects, the CRD is linked to an Fc component of human IgG1 (FcMBL).

In some embodiments of any of the aspects, the magnetic support is a superparamagnetic support.

In some embodiments of any of the aspects, the magnetic support comprises a magnetic bead, a superparamagnetic bead, or a magnetic microbead.

In some embodiments of any of the aspects, the engineered microbe-targeting molecule comprises FcMBL streptavidin linked to superparamagnetic beads.

In some embodiments of any of the aspects, the engineered microbe-targeting molecule comprises FcMBL linked to mesoporous silica particles.

In some embodiments of any of the aspects, the engineered microbe-targeting molecule is linked to an ELISA plate.

In some embodiments of any of the aspects, the microbe comprises a Gram-positive bacterial species, a Gram-negative bacterial species, a mycobacterium, a fungus, a parasite, or a virus.

In some embodiments of any of the aspects, the microbial component comprises a component from a Gram-positive bacterial species, a Gram-negative bacterial species, a mycobacterium, a fungus, a parasite, or a virus.

In some embodiments of any of the aspects, the virus is a coronavirus.

In some embodiments of any of the aspects, the virus is a severe acute respiratory syndrome coronavirus 2 (SARS-CoV-2).

In some embodiments of any of the aspects, the Gram-positive bacterial species comprises bacteria from the class Bacilli.

In some embodiments of any of the aspects, the Gram-negative bacterial species comprises bacteria from the class Gammaproteobacteria.

In some embodiments of any of the aspects, the mycobacterium comprises bacteria from the class Actinobacteria.

In some embodiments of any of the aspects, the fungus comprises fungus from the class Saccharomycetes.

In some embodiments of any of the aspects, the microbe is selected from the group consisting of Staphylococcus aureus, Streptococcus pyogenes, Klebsiella pneumoniae, Pseudomonas aeruginosa, Mycobacterium tuberculosis, Candida albicans, or Escherichia coli.

In some embodiments of any of the aspects, the microbe is a human pathogen.

In some embodiments of any of the aspects, the sample contains at least one pathogen.

In some embodiments of any of the aspects, the sample contains more than one pathogen.

In some embodiments of any of the aspects, the species of the pathogen is identified.

In some embodiments of any of the aspects, the strain of the pathogen is identified.

In some embodiments of any of the aspects, the drug sensitivity of the pathogen is identified.

In some embodiments of any of the aspects, the method further comprises providing a therapy model to the patient based on the infection category assigned to the patient.

In some embodiments of any of the aspects, the method further comprises providing a therapy model to the patient based on the identified pathogen assigned to the patient.

In some embodiments of any of the aspects, the therapy model comprises treatment with a therapeutic agent specific to the pathogen.

### BRIEF DESCRIPTION OF THE DRAWINGS

FIG. 1 is a schematic showing patient enrollment. Out of the 66 patient samples collected, 5 were removed due to the lack of availability of an at enrollment (time point 0 hr) prepared sample. Of the 61 patients remaining, 16 were classified as infection only (1 or no SIRS criteria met) and were excluded. Within those 45 patients remaining, 25 were classified as sepsis (infection+2 or more SIRS criteria met) and 19 were classified as severe sepsis (infection+1 or more organ dysfunctions) and 1 was classified as septic shock (infection+1 or more hypoperfusion criteria). From those, 17 patients were blood culture negative, but secondary culture information was available (wound, sputum, urine, or joint fluid) to indicate the source of infection. Of those 17 patients, 13 were culture positive with a confirmed or potential genus for which there was a developed library.

FIG. 2 is a schematic representation of spiked blood sample preparation. A solution of FcMBL-coated superparamagnetic beads is added to whole blood spiked with 1×10⁸ CFU/mL equivalent of prepared antibiotic-treated pathogen lysate for 30 minutes. During incubation, the FcMBL-coated beads capture pathogen-associated molecular patterns (PAMPs) found in the blood. The beads and their associated PAMPs are removed from the solution using a neodymium magnet. The beads are then washed several times and concentrated into a smaller volume of high performance liquid chromatography-grade (HPLC-grade) H₂O. The PAMPs are eluted off of the beads by subjecting them to 70° C. heat for 30 minutes on a bench top shaker. The supernatant containing the eluted material is removed and analyzed via MALDI-TOF MS and the methods described herein. Collectively, this procedure requires only 90 minutes from blood draw to sample identification.

FIG. 3A-3B show quantification of PAMPs through ELLecSA. FIG. 3A is a schematic showing an ELLecSA. An ELLecSA is a modified ELISA technique used to quantify the amount of PAMPs captured by FcMBL-coated beads. This technique was utilized to quantify samples prepared from spiked blood. FIG. 3B is a bar graph showing that pathogen samples contained 2 to 8-fold greater concentration of PAMPs/mL than pathogen-free controls. Due to differences in the composition of each pathogen lysate, FcMBL bound to a varying degree between different species, thus resulting in differences in sample concentration amongst species.

FIG. 4A-4B shows a representative mass spectrum produced from each species spiked into whole blood. FIG. 4A shows mass spectra in reflector positive mode. Of the 7 species, 6 could be visualized using the reflector positive mode with a m/z range of 700-5,000 Da. As shown herein, the mass spectra of a pathogen-free control and of samples prepared from S. aureus, S. pyogenes, K. pneumoniae, P. aeruginosa, C. albicans, and M. tuberculosis spiked into whole blood are represented.

FIG. 4B shows mass spectra in linear positive mode. Due to the composition of E. coli PAMPs captured by FcMBL-coated beads, this species could only be visualized by linear positive mode with a m/z range of 4,000-20,000 Da. All of the other species, with the exception of M. tuberculosis, could also be visualized within this size range of 4,000-20,000 Da. A collection of 50 spectra in both modes (where applicable) from 5 independent sample preparations was compiled into the fingerprint library of each species.

FIG. 5A-5G is a series of histograms showing a percent frequency representation of pathogen fingerprint libraries. Each fingerprint library is a compilation of 5 independent sample preparations which were used to produce 50 spectra in reflector positive and linear positive mode. Due to the lack of visualization of pathogen-specific peaks observed in one of the two modes, M. tuberculosis samples were only analyzed using reflector positive mode and E. coli samples were only analyzed using the linear positive mode. Each library was compared against an equally sized pathogen-free control library in order to remove background peaks associated with blood component interactions, sample processing, and reagents. The remaining pathogen-specific peaks were organized by clustering them into bins where each bin corresponds to a single molecule of interest. The percent frequency of each pathogen-specific peak within the total fingerprint library are represented in a bar graph. Fingerprint libraries were developed for S. aureus (FIG. 5A), S. pyogenes (FIG. 5B), K. pneumoniae (FIG. 5C), P. aeruginosa (FIG. 5D), C. albicans (FIG. 5E), E. coli (FIG. 5F), and M. tuberculosis (FIG. 5G).

FIG. 6 is a detailed schematic representation of unsupervised clustering algorithm and probabilistic model. The fingerprint libraries for controls, known reference pathogens, and the unknown were collected and clustered into bins where each bin corresponds to a single molecule of interest and is identified by the mean m/z value of that bin. All bins which were common between more than one library were ignored. The remaining bins which were found to be common between the unknown and exactly one library were weighted by frequency and trustworthiness. The weights of all bins were normalized to probabilities which sum to 1. The resulting output identified the probability that the unknown sample corresponded to one of the known pathogen fingerprint libraries. The legend for FIG. 6 is as follows. B represents the unknown entity, for example {L₀, L₁, x₂, . . . , [symbol] }. L₀ represents the control entity. L₁ represents pathogen library 1. L₂ represents pathogen library 2. Additional pathogen libraries can be designated by subscript notation. x represents a mass spectrum corresponding to one entity; for example, x_{B} represents an unknown spectrum, and x₀ represents a control spectrum. [symbol] refers to the entire library of spectra including controls; for example, [symbol] ={x₀, x₁, x₂, . . . , [symbol] }. N(i) represents the number of peaks in a bin i of peaks. N(i,x) represents the number of peaks of spectrum x in bin i. Tₘᵢₓ(p, τ) represents the trustworthiness weight for trustworthiness parameter τ and mixing proportion p.

FIG. 7A-7C is a series of graphs showing an explanation of trustworthiness. If p represents unknown proportion, 1-p represents pathogen library proportion, and τ is the level of trustworthiness. Note that in some embodiments of any of the aspects, Tₘᵢₓ(p, τ)+T_{unknown}(p, τ)+T_{library}(p, τ)=1, and the equation is solved for p. Curves represent Tₘᵢₓ(p, τ). Grey region describes range of unknown proportions to be trusted. See also below Formulas 1-3. T represents the trustworthiness weighting parameter.

$$\text{?} \qquad ( 1 )$$

$$\text{?} \qquad ( 2 )$$

$$\text{?} \qquad ( 3 )$$

$$\text{?} \text{indicates text missing or illegible when filed}$$

FIG. 8A-8J is a series of graphs showing diagnostic development in buffer. Pathogen samples were prepared in buffer first to verify whether each species can be visualized on the MALDI-TOF MS and whether consistent genera-specific pathogen fingerprint libraries can be developed to identify unknowns using the methods described herein. FIG. 8A is a bar graph showing the concentration of PAMPs present in samples prepared for each species in buffer quantified by ELLecSA. Pathogen samples were found to contain between 6 to 44-fold greater concentrations of PAMPs/mL than pathogen-free buffer controls. FIG. 8B shows characteristic spectra for all buffer samples analyzed using the reflector positive mode on the MALDI-TOF MS. E. coli samples could not be visualized in this mode. FIG. 8C shows characteristic spectra for all buffer samples analyzed using the linear positive mode on the MALDI-TOF MS. M. tuberculosis could not be visualized in this mode. FIG. 8D-8J is a series of bar graphs showing a percent frequency representation of all pathogen-specific m/z peaks from both modes comprising each buffer pathogen fingerprint library. Each of the fingerprint libraries contained 3 to 11 peaks occurring in at least 70% of the total spectra. A percent frequency representation is shown for S. aureus (FIG. 8D), S. pyogenes (FIG. 8E), K. pneumoniae (FIG. 8F), P. aeruginosa (FIG. 8G), C. albicans (FIG. 8H), E. coli (FIG. 8I), and M. tuberculosis (FIG. 8J).

FIG. 9A-9B is a series of representative mass spectra produced from bacteria treated with several different classes of antibiotics. To evaluate how changes in the mechanisms of action of different antibiotic classes can affect the spectra of a given species after FcMBL bead capture, samples were prepared for one Gram-positive and one Gram-negative bacteria, S. pyogenes and P. aeruginosa respectively. Each were treated with 4 different classes of species-appropriate antibiotics as described in Table 5, and samples were prepared for MALDI-TOF MS analysis in buffer. The resulting spectra in reflector positive mode for each species and antibiotic combination are shown in FIG. 9A-9B. FIG. 9A shows S. pyogenes cultures treated with lincomycin, daptomycin, vancomycin, and cefepime. The spectra of cultures prepared with cefepime, lincomycin, or vancomycin were the most similar. There were 19 m/z peaks shared between these samples, 14 of which occurred in over 50% of the total spectra. FIG. 9B shows P. aeruginosa cultures treated with amikacin, cefepime, ciprofloxacin, and meropenem. There were 10 m/z peaks found to be shared among all 4 classes of antibiotics. The two antibiotics found to produce the most similar spectra were ciprofloxacin and amikacin, whose samples shared 19 m/z peaks. Interestingly, the combined application of amikacin and cefepime (data not shown) produced a considerably greater amount of m/z peaks as compared to either antibiotic applied alone, demonstrating a potentially synergistic effect. Taken together, these results demonstrate the ability of this system to detect two different bacteria treated with several classes of antibiotics. Although there is a substantial amount of overlap across multiple or all antibiotics applied to each species, there is sufficient variation observed between antibiotic classes administered.

FIG. 10A-10B is a schematic and series of graphs showing a variation of the workflow that increases speed, sensitivity, and the number of identified microbes. FIG. 10A is a schematic of the workflow variation, wherein an asterisk (*) indicates a newly integrated step that increased speed and sensitivity. FIG. 10B is a pair of graphs showing example MALDI-TOF spectra for C. albicans, E. coli, K. pneumoniae, P. aeruginosa, S. aureus, and S. pyogenes using the variation of the workflow described in FIG. 10A and Example 2.

FIG. 11 is a bar graph comparing trypsin digestion methods. One-minute microwave trypsin digestion was compared to overnight trypsin digestion for S. aureus, K. pneumoniae, P. aeruginosa, S. pyogenes, C. albicans, E. coli, and a control. The concentration of PAMPs was determined through ELLecSA. Note that 1 min microwave digestion was comparable to overnight digestion, and different microbes were digested at different efficiencies. From left to right in the bar graph, the spiked pathogens are: Salmonella, Klebsiella, Pseudomonas, Streptococcus, E. coli, Candida, and control.

FIG. 12A-12H is a series of schematics showing a detailed workflow of the peak matching algorithm. FIG. 12A is a schematic showing a general overview of sample acquisition. FIG. 12B is a schematic showing a general workflow of the 5 steps of the peak matching algorithm. FIG. 12C is a schematic showing an example of Step 1, wherein the sample data is loaded into the algorithm. FIG. 12D is a schematic showing an example of Step 2a, wherein the m/z values are screened based on repeatability across a dataset and filtered according to the control library. FIG. 12E is a schematic showing an example of Step 2b, wherein the m/z values are filtered according the reference library. FIG. 12F is a schematic showing Step 3, wherein m/z values are combinatorially paired. FIG. 12G is a schematic showing Step 4, wherein the peak area ratios are compared. FIG. 12H is a schematic showing Step 5, wherein an output table is generated that compares the experimental sample to all pathogens in the library.

FIG. 13 depicts an example process and an example of an overview of a system according to some embodiments of the present disclosure. The top half of FIG. 13 is flowchart showing an example process for detecting a microbe or microbe component using mass spectrometry. In some embodiments of any of the aspects, a test sample 110 is received (for example from a subject 100). Additional samples can include control(s) 111 and reference(s) 112. In some embodiments of any of the aspects, the samples are optionally processed 115 to increase detection, as described further herein (see e.g., Example 2). The microbe or microbe components are then contacted and isolated 120 using an engineered microbe-targeting molecule linked to a support as described further herein. The isolated microbes or microbe components are then contacted 130 with a matrix or matrix solution as described further herein. The microbes or microbe components are then detected 140 using mass spectrometry as described further herein. The bottom half of FIG. 13 shows an example of an overview of a system according to some embodiments of the present disclosure. The isolated microbes or microbe components are input into a mass spectrometer 150, which is part of a system that includes a network 160, a computing device 170, a display 175, a server 180, and a database 185.

FIG. 14 is a flowchart showing an example process for identifying at least one microbe or microbe component in a sample using weighting parameters (see e.g., Example 1). First, dataset(s) are received 200 from the mass spectrometer. Then, the data points are clustered 210 into bins. Next, data points that match a control are removed 220. Then, weighting parameter(s) as described further herein are applied 230. A score is determined 240 comparing the sample to each reference. The microbe or microbe components in the sample is then identified 250 using the score from 240.

FIG. 15 is a flowchart showing an example process for identifying at least one microbe or microbe component in a sample using peak area ratio comparisons (see e.g., Example 3). First, dataset(s) are received 300 from the mass spectrometer. Then, the data points are clustered 310 into bins. Next, data points are filtered 320. Briefly, filtering 320 the data points can comprise at least one of the following: removing data point(s) with a repeatability value below a pre-determined threshold; removing data point(s) that match a control; and removing data point(s) that do not match the reference(s). Then, data points are paired and peak area ratios are determined 330 for the pairs. The peak area ratios are then compared 340 between the sample and reference(s). At least one score is output 350 based on the comparisons in 340. The microbe or microbe components in the sample is then identified 360 using the score(s) from 350.

FIG. 16 shows a series of MALDI-TOF MS spectra. Panel I shows MALDI-TOF MS spectrum of COVID-19 lentivirus pseudovirus captured on MPS-FcMBL beads. Panel II shows control spectrum of MBP-FcMBL beads without COVID. Panel III shows spectrum of COVID-19 pseudovirus spectrum of that was sonicated before capturing on MPS-FcMBL beads. Panel IV shows control spectrum of sonicated buffer.

### DETAILED DESCRIPTION

Embodiments of the technology described herein relate to methods for detection of microbes and microbe components. In some embodiments of any of the aspects, such methods can be applied to detect at least one microbe or at least one microbial component in a sample, including not limited to a patient sample, an animal model sample, an environmental sample, or a non-biological sample.

In some embodiments of any of the aspects, such methods can be applied to sepsis. Sepsis is a life-threatening condition caused by host systemic inflammatory responses to bloodstream infection. Microbial identification in blood using commercially available matrix-assisted laser desorption ionization time-of-flight mass spectrometry (MALDI-TOF MS) diagnostic systems is only applicable to the 15-30% of blood culture positive septic patients. Current standard-of-care with concurrent broad-spectrum antibiotics and low levels of live pathogen in the blood lead to the large majority of culture negative patients. Described herein are methods combining MALDI-TOF MS with immunomagnetic separation to rapidly identify fragmented pathogen components directly from whole blood. Accordingly, no culturing step is required, and in some embodiments the time from sample collection to the end of detection can take equal to or less than 90 minutes.

As described herein, a support (e.g., superparamagnetic beads, polymeric beads, mesoporous particles, etc.) coated with FcMBL, an engineered form of Mannose-Binding Lectin linked to the Fc immunoglobulin domain, are used to capture and concentrate pathogen-associated molecular patterns (PAMPs) from spiked blood. The collected materials are used to build genera-specific fingerprint libraries from the sample spectra produced by MALDI-TOF MS. From this information, an unsupervised clustering algorithm and probabilistic model was developed to predict unknown samples. Herein, the ability of this system to build unique fingerprint libraries is demonstrated for a diverse set of 7 relevant pathogens. These libraries were used as a reference to accurately identify spiked blood samples containing as little as 10 CFU/mL equivalent of antibiotic-treated pathogen. This system was applied to correctly identify clinical samples from blood culture negative septic patients in only 90 minutes from blood draw. This technique provides a rapid, culture-free diagnostic tool for septic patients, e.g., those undergoing standard-of-care with antibiotics, in which a positive identification cannot be obtained by current methods.

Accordingly, in one aspect, described herein is a method of detecting a microbe or microbe component, the method comprising the following steps: (i) contacting a sample with an engineered microbe-targeting molecule linked to a support; (ii) isolating the microbe or microbe components bound to the engineered microbe-targeting molecule; (iii) contacting the microbe or microbe components with a matrix or matrix solution on a target substrate; and (iv) detecting and/or analyzing the microbe or microbe components using a mass spectrometric method.

In another aspect, described herein is a method of detecting a microbial infection in a patient, the method comprising the following steps: (i) contacting a patient sample with an engineered microbe-targeting molecule linked to a support; (ii) isolating the microbe or microbe components bound to the engineered microbe-targeting molecule; (iii) contacting the microbe or microbe components with a matrix or matrix solution on a target substrate; and (iv) detecting and/or analyzing the microbe or microbe components using a mass spectrometric method.

Of note, the methods described herein comprise a step of isolating with an engineered microbe targeting molecule, which can eliminate the need for a culturing step. Furthermore, the methods described herein comprise a detection step, wherein the signal to noise ratio of data points obtained from a mass spectrometric method can be increased. In some embodiments of any of the aspects, the detection step comprises a clustering process and/or at least one weighting parameter, as described further herein. As used herein, a “clustering process” is a method that assigns each data point into a cluster. In some embodiments of any of the aspects, each cluster comprises a bin or cluster of data points from a single molecular signal of interest or from multiple molecular signals of interest. As used herein, “weighting parameter” can be a mathematical device used when performing a sum, integral, or average to give some elements more “weight” or influence on the result than other elements in the same set. As described further herein, non-limiting examples of weighting parameters include a frequency weighting parameter and a trustworthiness weighting parameter.

Also described herein are methods of producing a profile for a microbe, e.g., for the identification of at least one microbe or at least one microbe component in a sample. As used herein, a “profile” refers to a pattern of m/z data points or m/z clusters that can be used to identify at least one microbe or at least one microbe component in a sample. In some embodiments of any of the aspects, the results of the detection step comprise a profile, wherein said profile indicates the presence or absence of at least one microbe or microbe component. In some embodiments of any of the aspects, the profile is unique to a specific microbe or microbe component and as such can be used to identify an unknown microbe in a sample.

Described herein are methods of detecting at least one microbe or at least one microbial component in a sample, such as but not limited to, a patient sample. Such methods comprise a step of detecting and/or analyzing isolated microbe or microbe components with a mass spectrometric method.

In some embodiments of any of the aspects, the detection step comprises detecting the microbe or microbial component via a mass spectrometric method; analyzing the microbe or microbial component via a mass spectrometric method; performing a mass spectrometric method on the microbe or microbial component; identifying the microbe or microbial component via a mass spectrometric method; measuring the microbe or microbial component via a mass spectrometric method; and/or subjecting a mass spectrometric method on the microbe or microbial component.

Previous methods of detecting microbes with a mass spectrometric method can be limited by high background noise, especially in the presence of antimicrobials in a patient sample. Accordingly, detection methods as described herein can provide an increased signal to noise ratio through methods included but not limited to clustering and weighting data points, as described further herein.

In some embodiments of any of the aspects, the detecting step outputs mass spectrometric data obtained from the sample as a sample library. In some embodiments of any of the aspects, the detecting step outputs mass spectrometric data obtained from a control as a control library. In some embodiments of any of the aspects, the detecting step outputs mass spectrometric data obtained from a reference as a reference library.

In some embodiments of any of the aspects, the detection step comprises comparing the mass spectrometric data obtained from the sample (also referred to herein as a sample library or an experimental library) with at least one known reference library of mass spectrometric data (also referred to herein as a reference library) or with at least one known control library of mass spectrometric data (also referred to as a control library). Such comparisons can permit the identification of the at least one microbe in the sample. In some embodiments of any of the aspects, the sample library can comprise data obtained from at least 1, at least 2, at least 3, at least 4, at least 5, at least 6, at least 7, at least 8, at least 9, or at least 10 samples from the same patient or the same non-patient sample (i.e., biological replicates). A non-patient sample can comprise a sample taken from an animal model, from the environment, from a non-biological object, etc. In some embodiments of any of the aspects, the sample library can comprise data obtained from the same sample run on a mass spectrometer and/or detected at the same or different time points (i.e., technical replicates). In some embodiments of any of the aspects, data obtained from multiple reference samples can be combined, summed, and/or averaged to yield one sample library for each patient or non-patient sample. In some embodiments of any of the aspects, multiple sample libraries can be generated comprising a library from each of at least two different patients or at least two non-patient samples.

In some embodiments of any of the aspects, the reference library comprises data obtained from at least one sample comprising at least one known microbe or known components of at least one known microbe. In some embodiments of any of the aspects, the reference library can comprise data obtained from at least 1, at least 2, at least 3, at least 4, at least 5, at least 6, at least 7, at least 8, at least 9, at least 10, at least 20, at least 30, at least 40, at least 50, or at least 100 samples of the same known microbe species, the same known microbe strain, the same set of at least two known microbe species or strains, the same known microbe component, or set of at least two known microbe components. In some embodiments of any of the aspects, the reference library can comprise samples from the same patient, different patients, the same non-patient sample, or different non-patient samples. In some embodiments of any of the aspects, the reference library can comprise data obtained from the same reference sample run on a mass spectrometer and/or detected at the same or different time points. In some embodiments of any of the aspects, data obtained from multiple reference samples can be combined, summed, and/or averaged to yield one reference library for each known microbe, known microbe strain, set of at least two known microbe species or strains, known microbe component, or set of at least two known microbe components.

In some embodiments of any of the aspects, at least two reference libraries can be obtained, corresponding to at least two different species of known microbes, or at least two different strains of known microbes, or at least two different sets each comprising at least two known microbe species or strains, or at least two different known microbe components, or at least two different sets of at least two known microbe components. In some embodiments of any of the aspects, at least two reference libraries can be obtained, corresponding to the same species of known microbes, the same strains of known microbes, the same set of at least two known microbe species or strains, the same known microbe component, or the same set of at least two microbe components, but comprising different sampling and/or processing conditions. In some embodiments of any of the aspects, reference libraries can be obtained from least 2, at least 3, at least 4, at least 5, at least 6, at least 7, at least 8, at least 9, at least 10, at least 20, at least 30, at least 40, at least 50, or at least 100 known microbe species, known microbe strains, or sets of at least two known microbe species or strains.

In some embodiments of any of the aspects, the detection step comprises comparing the sample library with at least one control library. In some embodiments of any of the aspects, the control library comprises data obtained from at least one control sample comprising the same sampling conditions and/or processing conditions as the reference samples, patient samples, or non-patient samples. In some embodiments of any of the aspects, the control library comprises the same components as the reference samples, patient samples, or non-patient samples, but lacks or substantially lacks at least one microbe or at least one microbe component. In some embodiments of any of the aspects, the control library comprises the same components as the reference samples, patient samples, or non-patient samples, but lacks or substantially lacks microbes and microbe components.

As a non-limiting example, a control sample can comprise a sample from a control, such as but not limited to a healthy patient, an uninfected patient, an uninfected animal model, or an uninfected environmental sample. In some embodiments of any of the aspects, the control library can comprise at least 2, at least 3, at least 4, at least 5, at least 6, at least 7, at least 8, at least 9, at least 10, at least 20, at least 30, at least 40, at least 50, or at least 100 control samples. In some embodiments of any of the aspects, the control library can comprise data obtained from the same control sample run on a mass spectrometer and/or detected at the same or different time points. In some embodiments of any of the aspects, data obtained from multiple control samples can be combined, summed, and/or averaged to yield one control library. In some embodiments of any of the aspects, multiple control libraries (e.g., at least 2, at least 3, at least 4, at least 5, at least 10 control libraries) can be obtained, such as those comprising different sampling and/or processing conditions.

In some embodiments of any of the aspects, each of the sample libraries, the reference libraries, and/or the control libraries are obtained and/or detected under the same conditions (e.g., patient/model conditions, microbe isolation, sample preparation, mass spectrometric methods, detection methods, analysis methods, etc., as described herein). In some embodiments of any of the aspects, the reference library is obtained from a patient or a model treated with the same drug or drug class as the sample library and/or control library. In some embodiments of any of the aspects, the reference library is an average or composite obtained from multiple patients or models, representing collectively, treatment with multiple drugs and/or drug classes. In some embodiments of any of the aspects, the average/composite reference library includes the drug or drug class used to treat the instant patient or model. In some embodiments of any of the aspects, at least one sample library, at least one reference library, and/or at least one control library is obtained and/or detected with the same patient/model conditions and/or using the same methods of microbe isolation, sample preparation, mass spectrometry, detection, and/or analysis, etc., as described herein).

In some embodiments of any of the aspects, the detection step comprises comparing at least one sample library with at least one reference library or at least one control library. In some embodiments of any of the aspects, the detection step comprises comparing at least one sample library with at least one reference library and at least one control library.

In some embodiments of any of the aspects, the step of detecting comprises analyzing a set of data output from a mass spectrometer with a control system comprising one or more processors. A processor encompasses all kinds of apparatus, devices, and machines for processing data, including by way of example a programmable processor, a computer, a system on a chip, or multiple ones, or combinations, of the foregoing. The control system and one or more processors may be local or remotely located on a server with the results stored in a database. In some embodiments, the analyzing and/or detecting the data may be performed using a combination of local and remote processors, for instance by first preprocessing the data locally. A control system comprises at least one processor (e.g., at least 1, at least 2, at least 3, at least 4, at least 5 processors) and can optionally comprise a user interface, a display, and/or at least one port for or means of interfacing with a mass spectrometer (e.g., for obtaining data points from a mass spectrometric method).

In some embodiments of any of the aspects, the data points from a mass spectrometric method comprise m/z peaks. As used herein, the term “m/z” refers to the mass-to-charge ratio of a cation (also referred to herein as m/e or “mass ratio”), which is equal to the mass of the cation divided by its charge. In some embodiments wherein the charge of cation formed in a mass spectrometer is +1, the mass-to-charge ratio of a cation is equal to the mass of the cation. Mass ratio data can be displayed in a mass spectrum, which is an intensity vs. m/z (mass-to-charge ratio) plot. The mass spectrum of a sample is a pattern representing the distribution of mass-to-charge ratio in a sample. The mass spectrum (also referred to herein as a profile or fingerprint) can be unique to specific sample or analyte (e.g., microbe or microbe component) and as such can be used to identify an unknown microbe in a sample. The term “m/z peak” refers to a data point at a specific m/z value (e.g., x-axis of a mass spectrum) of a specific intensity (e.g., y-axis of a mass spectrum; also referred to herein as relative intensity or abundance) in a mass spectrum. Described herein are methods of detecting and/or analyzing m/z peaks, e.g., from isolated microbes and microbe components for the detection of said microbes and microbe components.

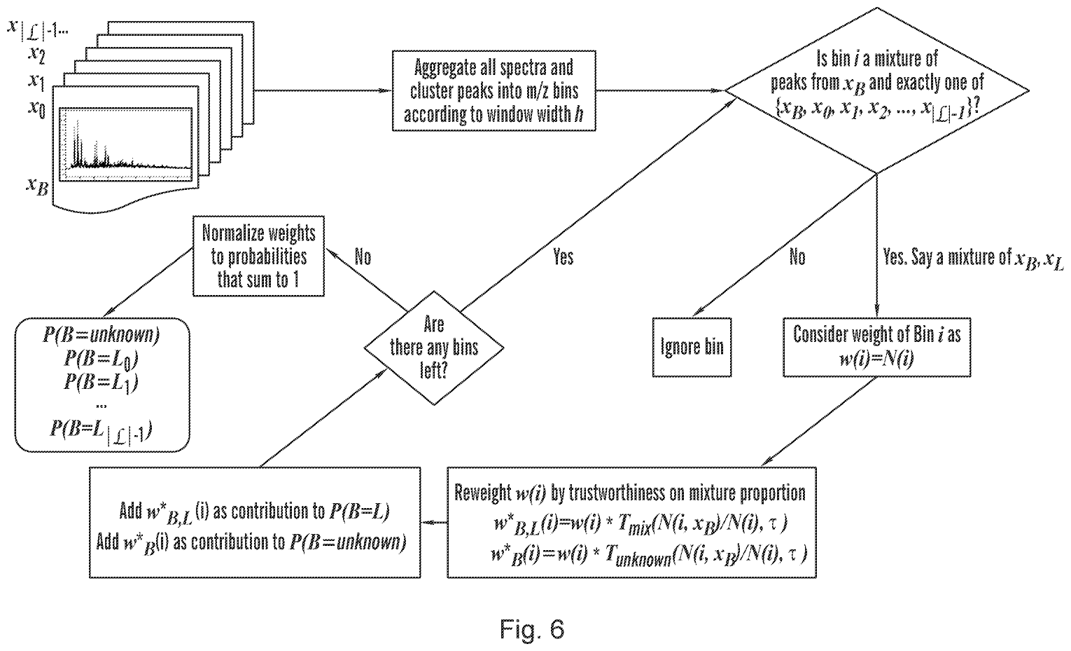

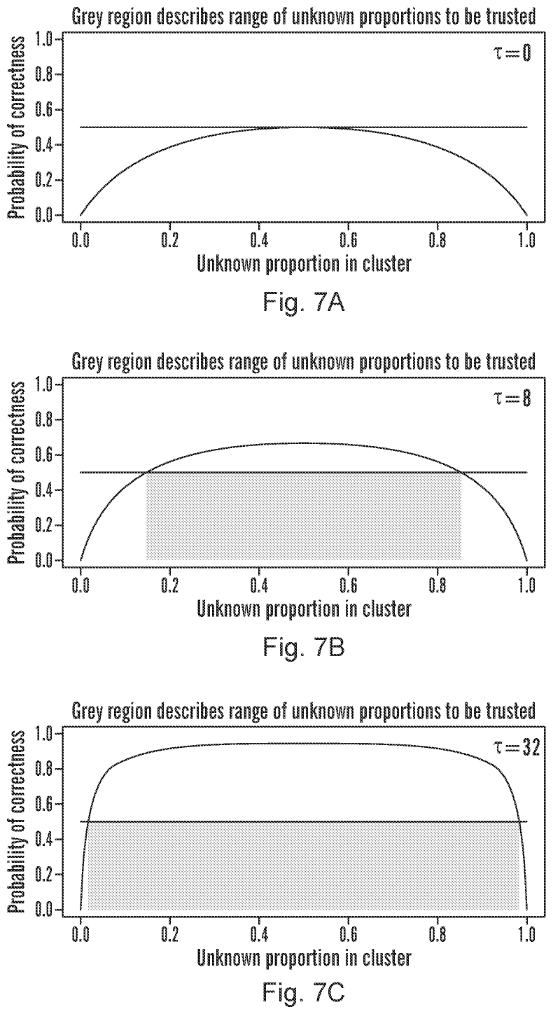

In some embodiments of any of the aspects, the control system is configured to execute machine executable code. Machine executable code (also known as a computer program, program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. The machine executable code can comprise algorithms as described herein (see e.g., Example 1, FIG. 6, FIG. 7A-7C).

In some embodiments of any of the aspects, the control system is configured to execute machine executable code using a clustering process, as described herein. As used herein, a “clustering process” is a method that assigns each data point into a cluster. In some embodiments of any of the aspects, each cluster comprises a bin or cluster of data points from a single molecular signal of interest or from multiple molecular signals of interest. As used herein, “molecular signal of interest” refers to a single molecule or group of closely related molecules (i.e., molecules of similar mass-to-charge ratio) including but not limited to a polypeptide, peptide, nucleic acid, oligonucleotide, lipid, lipoprotein, small molecule, microbe, microbe component, microbe-associated molecular pattern (MAMP), pathogen-associated molecular pattern (PAMP), an antimicrobial-resistance-associated molecule, an antimicrobial-susceptibility-associated molecule, and the like.

In some embodiments of any of the aspects, the clustering of data points from a mass spectrometric method is based on m/z peaks identified by the maximum intensity of that cluster. As used herein, “maximum intensity” refers to the m/z value with the highest abundance (e.g., y-axis of a mass spectrum) of all m/z peaks in a cluster or bin. In some embodiments of any of the aspects, the clustering of data points from a mass spectrometric method is based on m/z peaks identified by the mean m/z value of that cluster. As used herein, “mean m/z value” refers to the average m/z value (e.g., x-axis of a mass spectrum) of all m/z peaks in a cluster or bin.

In some embodiments of any of the aspects, at least 2 data points (e.g., m/z peaks from a single molecular signal of interest) are clustered into a bin or cluster. As a non-limiting example, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 data points (e.g., m/z peaks from at least one molecular signal of interest) are clustered into a bin or cluster.

In some embodiments of any of the aspects, the data points in at least one sample library are clustered. In some embodiments of any of the aspects, the data points in in at least one control library are clustered. In some embodiments of any of the aspects, the data points in in at least one reference library are clustered. In some embodiments of any of the aspects, the data points in in at least one sample library and at least one control library are clustered, e.g., using the same parameters. In some embodiments of any of the aspects, the data points in in at least one sample library and at least one reference library are clustered, e.g., using the same parameters. In some embodiments of any of the aspects, the data points in in at least one control library and at least one reference library are clustered, e.g., using the same parameters. In some embodiments of any of the aspects, the data points in in at least one sample library, at least one control library, and at least one reference library are clustered, e.g., using the same parameters.

In some embodiments of any of the aspects, a cluster is defined by a window width “h”, or a range of m/z values. In some embodiments of any of the aspects, all clusters are the same width, i.e., are defined by the same h. In some embodiments of any of the aspects, a first subset of clusters is the same width (i.e., are defined by the same h₁), a second subset of clusters is the same width (i.e., are defined by the same h₂), a third subset of clusters is the same width (i.e., are defined by the same h₃), etc. As used herein, a “subset of clusters” or a “subset of bins” can comprise clusters or bins that are of contiguous m/z values or clusters or bins that are of non/contiguous m/z values. In some embodiments of any of the aspects, all samples, controls, and references are processed using the same h or subset of h (h₁, h₂, h₃, etc.).

In some embodiments of any of the aspects, the window width h of a cluster is at least 1 Dalton (Da), i.e., each cluster of data points is at least 1.0 Dalton (Da) wide. The mass-to-charge ratio unit (m/z) can be used interchangeably with Dalton. In some embodiments of any of the aspects, a mass difference is measured in parts per million (ppm). As a non-limiting example, each cluster of data points is at least +/−0.1 Da, at least +/−0.2 Da, at least +/−0.3 Da, at least +/−0.4 Da, at least +/−0.5 Da, at least +/−0.6 Da, at least +/−0.7 Da, at least +/−0.8 Da, at least +/−0.9 Da, at least +/−1.0 Da, at least +/−1.1 Da, at least +/−1.2 Da, at least +/−1.3 Da, at least +/−1.4 Da, at least +/−1.5 Da, at least +/−1.6 Da, at least +/−1.7 Da, at least +/−1.8 Da, at least +/−1.9 Da, at least +/−2.0 Da, at least +/−2.5 Da, at least +/−3.0 Da, at least +/−3.5 Da, at least +/−4.0 Da, at least +/−4.5 Da, at least +/−, at least +/−5.0 Da wide, at least +/−5.5 Da, at least +/−6.0 Da, at least +/−6.5 Da, at least +/−7.0 Da, at least +1-7.5 Da, at least +/−8.0 Da, at least +/−8.5 Da, at least +/−, at least +/−9.0 Da, at least +/−9.5 Da, or at least +/−10.0 Da wide.

In some embodiments of any of the aspects, the window width h of a cluster can depend on the mass range being measured. In some embodiments of any of the aspects, smaller masses (e.g., less than 5000 Da) can be clustered using a window width h of +/−1 Da, +/−1 m/z, or 50 ppm. In some embodiments of any of the aspects, larger masses (e.g., equal to or greater than 5000 Da) can be clustered using a window width h of +/−10 Da, +/−10 m/z, or 500 ppm.

In some embodiments of any of the aspects, at least 2 data points (e.g., m/z peaks of a mass spectrum) can be processed into at least 1 cluster or bin. As a non-limiting example, a mass spectrum can be processed into or can comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 clusters or bins. In some embodiments of any of the aspects, the clustering process can be applied to all data points. In some embodiments of any of the aspects, the clustering process can be applied to a subset of data points. As used herein, a “subset of data points” can comprise data points that are of contiguous m/z values or data points that are of non-contiguous m/z values.

In some embodiments of any of the aspects, after the step of applying a clustering process, at least one sample library or at least one reference library is compared to at least one control library. In some embodiments of any of the aspects, the clusters of a sample library are compared to the clusters of a control library. In some embodiments of any of the aspects, a cluster of a sample library is removed or decreased (e.g., using a weighting parameter) if it is shared with the same cluster of a control library. In some embodiments of any of the aspects, the clusters of a reference library are compared to the clusters of a control library. In some embodiments of any of the aspects, a cluster of a reference library is removed or decreased (e.g., using a weighting parameter) if it is shared with the same cluster of a control library. In some embodiments of any of the aspects, the clusters of a sample library are compared to the clusters of a reference library. In some embodiments of any of the aspects, a cluster of a sample library is removed or decreased (e.g., using a weighting parameter) if it is shared with the same cluster of at least two (e.g., at least 2, at least 3, at least 4, at least 5) reference libraries. Such comparisons are one method of reducing background noise (e.g., removing m/z peaks that correspond to non-microbe molecules) and can increase the signal to noise ratio of a sample library or a reference library.

In some embodiments of any of the aspects, the detection process comprises determining a repeatability value for at least one data point. In some embodiments of any of the aspects, a library (e.g., an experimental library, a reference library, a control library) comprises at least 2 replicates (e.g., biological or technical replicates). As a non-limiting example, a library (e.g., an experimental library, a reference library, a control library) comprises at least 2, 3, 4, 5, 6, 7, 8, 9, or at least 10 replicates (e.g., biological or technical replicates). In some embodiments of any of the aspects, a repeatability value (also referred to as “per_match”) is determined for at least one data point in at least one library. As used herein, the term “repeatability value” is the fraction of replicates from a library comprising a specific data point and is determined using formula 7 below.

“repeatability value”=(number of experimental replicates of a sample that contain a particular m/z value)/(number of experimental replicates) (7)

In some embodiments of any of the aspects, the detection process comprises removing at least one data point of the sample library, wherein the at least one data point comprises a repeatability value at or below a pre-determined threshold. In some embodiments of any of the aspects, the detection process comprises removing at least one data point of the control library, wherein the at least one data point comprises a repeatability value at or below a pre-determined threshold. In some embodiments of any of the aspects, the detection process comprises removing at least one data point of the reference library, wherein the at least one data point comprises a repeatability value at or below a pre-determined threshold. In some embodiments of any of the aspects, the pre-determined threshold for the repeatability value is 0.4. In some embodiments of any of the aspects, the pre-determined threshold for the repeatability value is at least 0.1, 0.2, 0.3, 0.35, 0.4, 0.45, 0.5, 0.6, 0.7, 0.8, 0.9, or 1.0. In some embodiments of any of the aspects, the detection process comprises removing each data point of the sample library, control library, and reference library, wherein the data point comprises a repeatability value at or below a pre-determined threshold (e.g., 0.4).

In some embodiments of any of the aspects, the clustering process further comprises removing at least one data point of the sample library that matches a data point in a control library within +/−0.3 Da. In some embodiments of any of the aspects, the clustering process further comprises removing at least one data point of the sample library that matches a data point in a control library. In some embodiments of any of the aspects, the clustering process further comprises removing at least one cluster of the sample library that matches a cluster in a control library. In some embodiments of any of the aspects, the entire bin/cluster is removed and one or more m/z peaks (e.g., that are within the bin/cluster) are removed from a library. Each bin/cluster comprises m/z values that are within a preset window width (e.g., +/−0.3 Da). If two or more m/z values are within the preset window width (e.g., +/−0.3 Da), those peaks are clustered under one m/z value. Decreasing the bin/cluster criteria to less than the preset window width (e.g., <+/−0.3 Da) reduces the likelihood of removing more than one m/z peak. In some embodiments of any of the aspects, one or more m/z peaks are removed if they are within a preset window width (e.g., +/−0.3 Da), but only one bin/cluster is removed.

As used herein, the term “match” (which is used interchangeably with correspond) refers to a data point or cluster defined by an m/z value (e.g., the maximum m/z value of a cluster; e.g., the mean m/z value of a cluster) that is within a pre-set m/z value distance (e.g., +/−0.3 Da) from a data point or cluster in another library. In some embodiments of any of the aspects, at least one data point (or cluster) of the sample library is removed that matches a data point (or cluster) in a control library within +/−0.3 Da. In some embodiments of any of the aspects, at least one removed data point (or cluster) of the sample library matches a data point (or cluster) in a control library within at least −2.0 Da, at least −1.5 Da, at least −1.0 Da, at least −0.9 Da, at least −0.8 Da, at least −0.7 Da, at least −0.6 Da, at least −0.5 Da, at least −0.4 Da, at least −0.3 Da, at least −0.2 Da, at least −0.1 Da, at least 0 Da, at least 0.1 Da, at least 0.2 Da, at least 0.3 Da, at least 0.4 Da, at least 0.5 Da, at least 0.6 Da, at least 0.7 Da, at least 0.8 Da, at least 0.9 Da, at least 1.0 Da, at least 1.5 Da, or at least 2.0 Da. In some embodiments of any of the aspects, the total number of data points (or clusters) in a sample library that do not match a corresponding data point (or cluster) in the control library is determined.

In some embodiments of any of the aspects, the clustering process further comprises removing at least one data point of the sample library that does not match a data point in at least one reference library within +/−0.3 Da. In some embodiments of any of the aspects, the clustering process further comprises removing at least one data point of the sample library that does not match a data point in at least one reference library. In some embodiments of any of the aspects, the clustering process further comprises removing at least one cluster of the sample library that does not match a cluster in at least one reference library. In some embodiments of any of the aspects, the clustering process further comprises removing at least one data point (or cluster) of the sample library that does not match a data point (or cluster) in any reference library of any known microbe or microbe component. In some embodiments of any of the aspects, at least one data point (or cluster) of the sample library is removed that does not match a data point (or cluster) in at least one reference library within +/−0.3 Da. In some embodiments of any of the aspects, at least one removed data point (or cluster) of the sample library does not match a data point (or cluster) in at least one reference library within at least −2.0 Da, at least −1.5 Da, at least −1.0 Da, at least −0.9 Da, at least −0.8 Da, at least −0.7 Da, at least −0.6 Da, at least −0.5 Da, at least −0.4 Da, at least −0.3 Da, at least −0.2 Da, at least −0.1 Da, at least 0 Da, at least 0.1 Da, at least 0.2 Da, at least 0.3 Da, at least 0.4 Da, at least 0.5 Da, at least 0.6 Da, at least 0.7 Da, at least 0.8 Da, at least 0.9 Da, at least 1.0 Da, at least 1.5 Da, or at least 2.0 Da. In some embodiments of any of the aspects, the total number of data points (or clusters) in a sample library that match a corresponding data point (or cluster) in a specific reference library is determined.

In some embodiments of any of the aspects, the detection step comprises determining a peak area ratio of at least one pair of data points within the sample library. In some embodiments of any of the aspects, the detection step comprises determining a peak area ratio of at least one pair of data points in at least one reference library. In some embodiments of any of the aspects, the detection step comprises determining a peak area ratio of at least one pair of data points within the sample library and at least one pair of data points in at least one reference library. In some embodiments of any of the aspects, the detection step comprises determining a peak area ratio of at least one pair of clusters within the sample library. In some embodiments of any of the aspects, the detection step comprises determining a peak area ratio of at least one pair of clusters in at least one reference library. In some embodiments of any of the aspects, the detection step comprises determining a peak area ratio of at least one pair of clusters within the sample library and at least one pair of clusters in at least one reference library.

As used herein, the term “peak area ratio” (PAB) is determined using formula 4 below, wherein “AreaA” is the area of a first peak from a dataset and “AreaB” is the area of a second peak from the same dataset as AreaA. In some embodiments of any of the aspects, the area of a peak (or cluster) is determined by the area under the curve and can be determined using methods as known in the art.

$$= \frac{{Area}_{B}}{{Area}_{B} + {Area}_{A}} \times 100 \qquad ( 4 )$$

As used herein, the term “pairs” (used interchangeably with “m/z pairs”) refers to a group of two data points (e.g., m/z peaks) or to a group of two clusters within in one library (e.g., sample library, reference library, control library). As used herein, the term “all possible pairs” refers to each potential pairing of data points or clusters in a library. As a non-limiting example, if a library consists of peak A, peak B, and peak C, then all possible pairs are: peak A and peak B; peak A and peak C; and peak B and peak C. In some embodiments of any of the aspects, the detection step comprises determining a peak area ratio of all possible pairs of data points (or clusters) within the sample library and all possible pairs of data points (or clusters) in at least one reference library. In some embodiments of any of the aspects, at least one peak area ratio of the sample library is compared to at least one corresponding (i.e., matching) peak area ratio of at least one reference library. In some embodiments of any of the aspects, each peak area ratio of the sample library is compared to each corresponding (i.e., matching) peak area ratio of each reference library. In some embodiments of any of the aspects, a percent difference is calculated between at least one peak area ratio of the sample library and at least one corresponding (i.e., matching) peak area ratio of at least one reference library. In some embodiments of any of the aspects, a percent difference is calculated between each peak area ratio of the sample library and each corresponding (i.e., matching) peak area ratio of each reference library. In some embodiments of any of the aspects, “percent difference” is determined using formula 5 below, wherein “PAR_{S}” refers to the peak area ratio of a data point (or cluster) in a sample library, PAR_{R}” refers to the peak area ratio of the corresponding data point (or cluster) in a reference library, and the percent difference is determined by the absolute value of the difference between PAR_{S} and PAR_{R} over PAR_{S}, expressed as a fraction or multiplied by 100 as a percentage.

$$\frac{\vert {PAR}_{R} - P A {R}_{S} \vert}{( P A {R}_{S} )} \qquad ( 5 )$$

In some embodiments of any of the aspects, a “conclusion” is determined as true or false for each PAR in a sample library based on the percent difference calculation (e.g., Formula 5). In some embodiments of any of the aspects, a “conclusion” is determined as true for a PAR in a sample library if the percent difference is at or below a pre-determined threshold (e.g., 15%). In some embodiments of any of the aspects, a “conclusion” is determined as true for a PAR in a sample library if the percent difference is at or below 15%. In some embodiments of any of the aspects, a “conclusion” is determined as false for a PAR in a sample library if the percent difference is above a pre-determined threshold (e.g., 15%). In some embodiments of any of the aspects, a “conclusion” is determined as false for a PAR in a sample library if the percent difference is above 15%. In some embodiments of any of the aspects, the pre-determined threshold is a percent difference between a sample PAR and a corresponding PAR in a reference library of at least 5%, at least 10%, at least 11%, at least 12%, at least 13%, at least 14%, at least 15%, at least 16%, at least 18%, at least 18%, at least 19%, at least 20%, at least, or at least 25%.

In some embodiments of any of the aspects, a score (e.g., an Area Score) is calculated based on the comparison of at least one peak area ratio of the sample library and at least one corresponding peak area ratio of at least one reference library. In some embodiments of any of the aspects, a score (e.g., an Area Score) is calculated based on the comparison of each peak area ratio of the sample library and each corresponding peak area ratio of the reference library of each known microbe or microbe component. In some embodiments of any of the aspects, if a conclusion is determined as true for a PAR in a sample library (e.g., percent difference at or less than 15%), then the Area Score is zero (0). In some embodiments of any of the aspects, if a conclusion is determined as false for a PAR in a sample library (e.g., percent difference above 15%), then an Area Score is determined for the PAR. As used herein, the term “Area Score” (also referred to herein as a “Difference Score” or a “Peak Area Difference Score”) is determined using formula 6 below, wherein “PAR_{S}” refers to the peak area ratio of a data point (or cluster) in a sample library, PAR_{R}” refers to the peak area ratio of the corresponding data point (or cluster) in a reference library, and the score is determined by the absolutely value of the difference between PAR_{S} and PAR_{R}. In other words, Peak Area Difference Score=(Peak Area Ratio of the Experimental Dataset)−(Peak Area Ratio of the Library Dataset).

|PAR_{S}−PAR_{R}| (6)

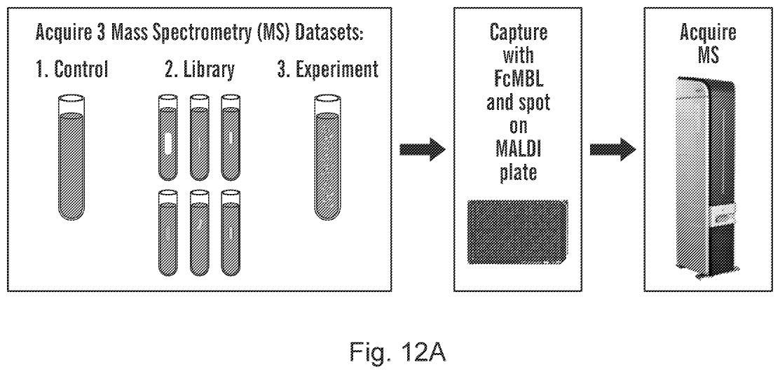

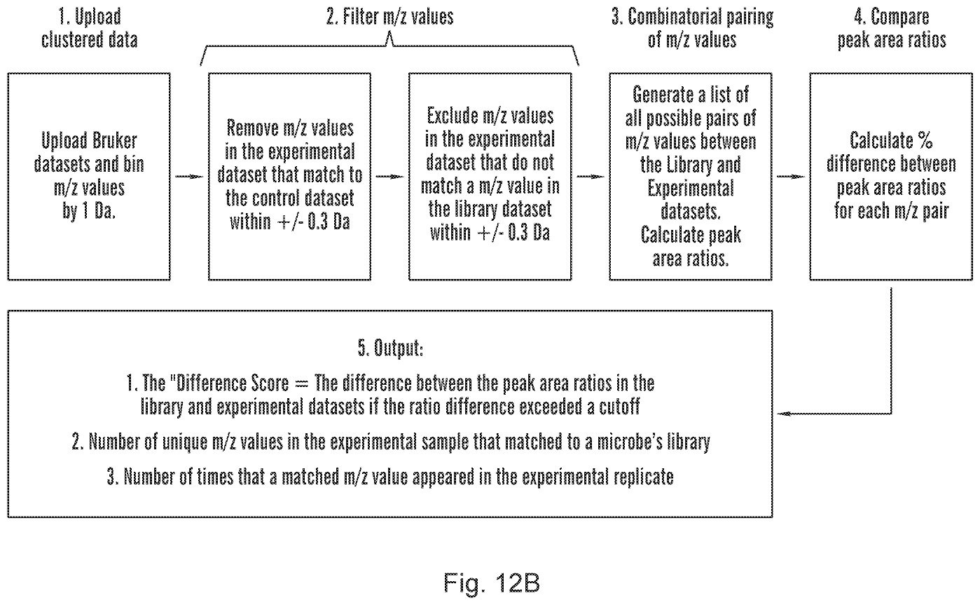

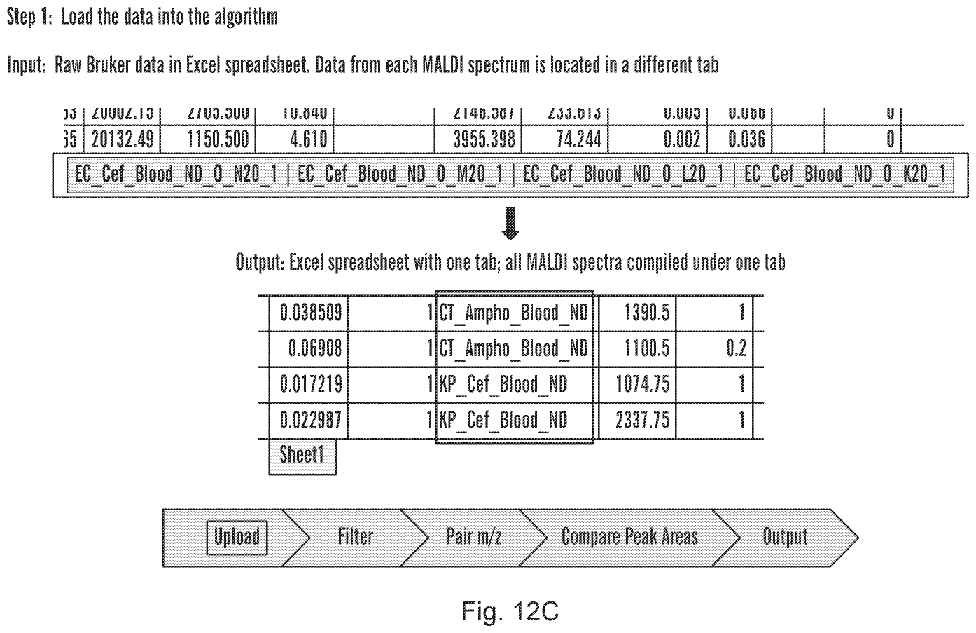

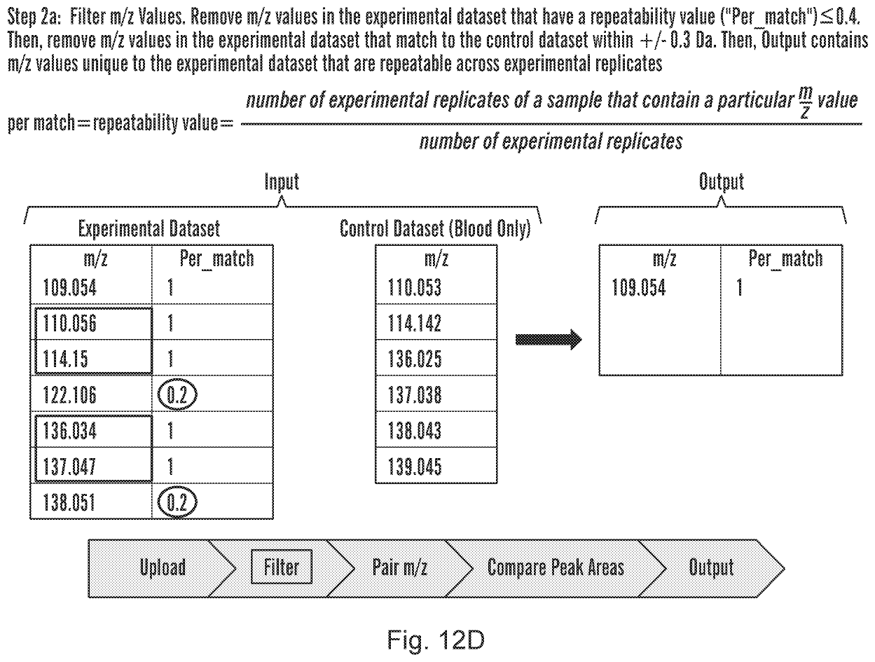

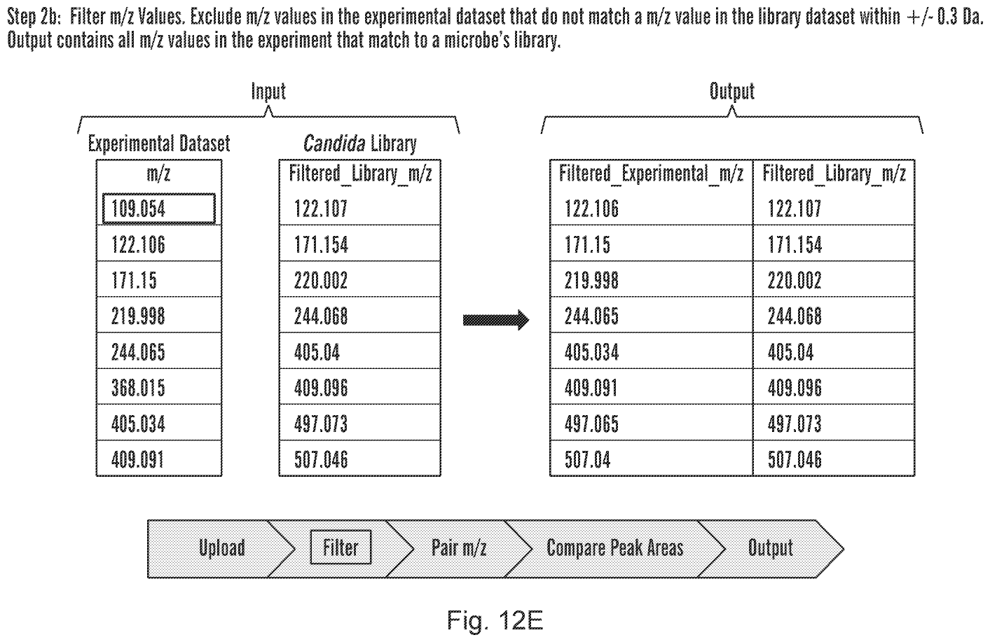

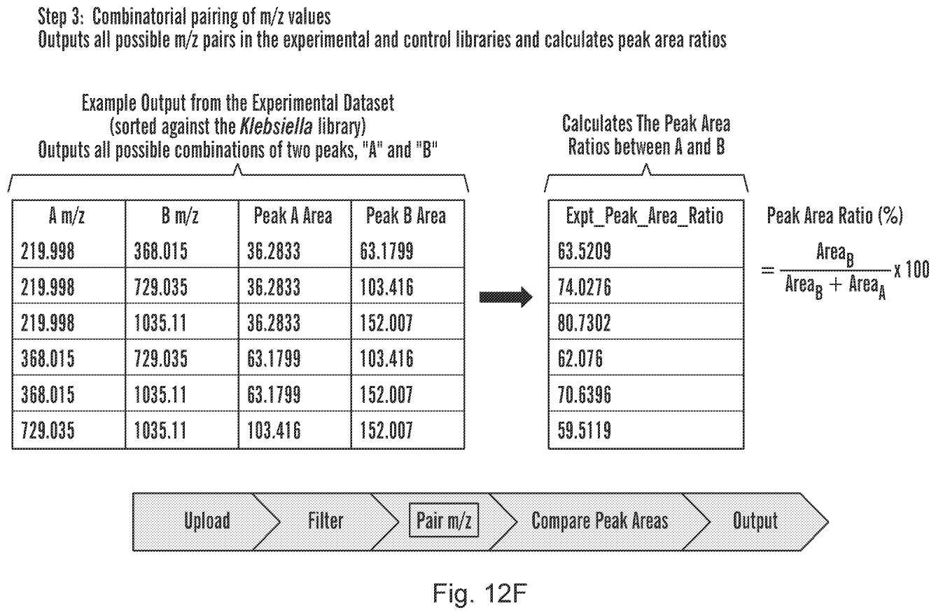

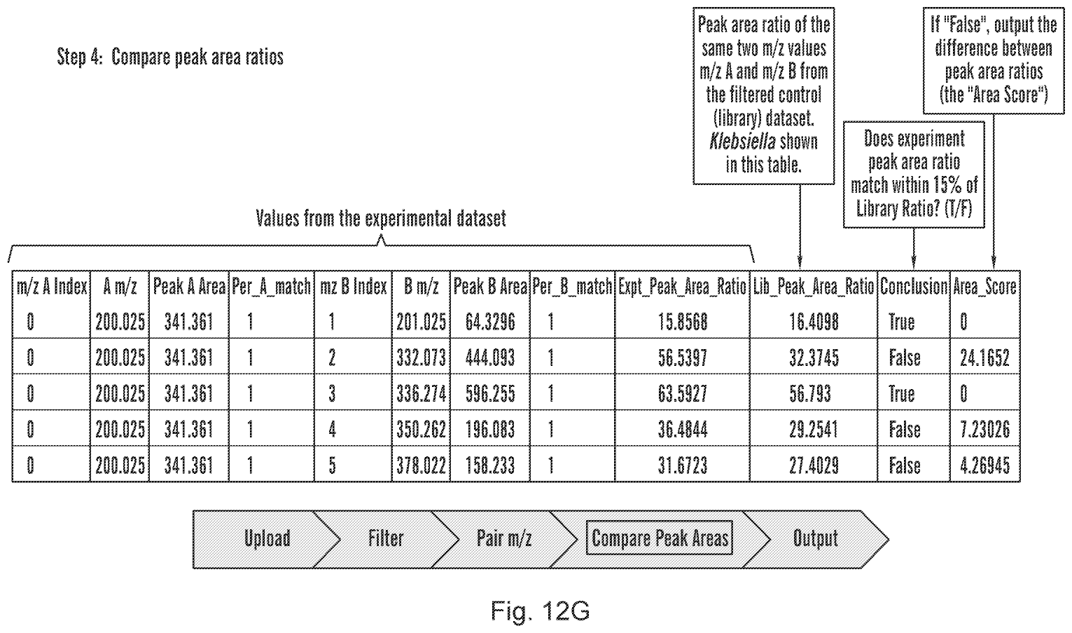

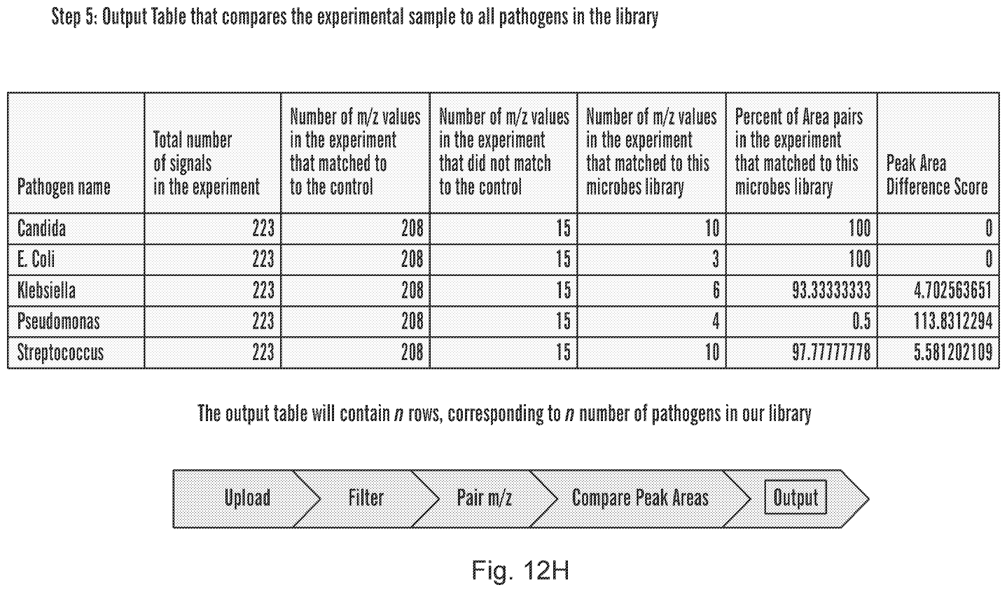

In some embodiments of any of the aspects, the sample library is compared to a reference library of each known microbe or microbe component using at least one of the following: (1) the total Area Score; (2) the number of data points (or clusters) in the sample library that match (e.g., within +/−0.3 Da) a corresponding data point or cluster in each reference library; or (3) the percent of area pairs that match to each reference library. In some embodiments of any of the aspects, a “total area score” is calculated by summing the area score of every PAR (e.g., FIG. 12G-12H).

In some embodiments of any of the aspects, at least one microbe in the sample library is identified as matching a specific reference library based at least one of the following: (a) the reference library with the lowest total Area Score (e.g., as close to zero as possible) of all known reference libraries; (b) the reference library with the highest number of data points (or clusters) that match (e.g., within +/−0.3 Da) the sample library, out of all known reference libraries; and/or (c) the reference library with the highest percentage (e.g., as close to 100% as possible) of PAR_{S} that match (e.g., within 15%) the sample library, out of all known reference libraries.

In some embodiments of any of the aspects, the clustering process further comprises applying a weighting parameter. As used herein, “weighting parameter” (also referred to as a “weight function” or “weight parameter”) can be a mathematical device used when performing a sum, integral, or average to give some elements (e.g., data points or clusters) more “weight” or influence on the result than other elements in the same set (e.g., mass spectrum or profile). In some embodiments of any of the aspects, the weight of a data point can refer to the intensity of an m/z peak, e.g., a weighting parameter can increase or decrease the intensity of at least one m/z peak. As a non-limiting example, the result of this application of a weighting parameter can be a weighted sum or weighted average. As described herein, a weighting parameter can permit the identification of the proportion of data points that are unique to the sample or common with a reference library or a control library.

In some embodiments of any of the aspects, the detection method comprises a clustering process or a weighting parameter. In some embodiments of any of the aspects, the detection method comprises a clustering process. In some embodiments of any of the aspects, the detection method comprises a weighting parameter. In some embodiments of any of the aspects, the detection method comprises a clustering process and a weighting parameter. In some embodiments of any of the aspects, the clustering process is applied before the weighting parameter is applied. In some embodiments of any of the aspects, the weighting parameter is applied before the clustering process is applied. In some embodiments of any of the aspects, the clustering process and the weight parameter are applied simultaneously. In some embodiments of any of the aspects, the clustering process and/or the weight parameter are applied iteratively.

In some embodiments of any of the aspects, the clustering process is applied to the same data points, clusters, or bins as the weighting parameter. In some embodiments of any of the aspects, the clustering process is applied to a first subset of data points, clusters, or bins, and the weighting parameter is applied to a second subset of data points, clusters, or bins, wherein the first subset and second subset comprise shared data points, clusters, or bins. In some embodiments of any of the aspects, the clustering process is applied to a first subset of data points, clusters, or bins, and the weighting parameter is applied to a second subset of data points, clusters, or bins, wherein the first subset and second subset do not comprise shared data points, clusters, or bins.

In some embodiments of any of the aspects, the weighting parameter comprises a frequency weighting parameter or a trustworthiness weighting parameter. As used herein, “frequency weighting parameter” refers to a parameter (e.g., a numerical variable) that increases the weight of a data point if the cluster containing the data point comprises additional other data points. As used herein, “trustworthiness weighting parameter” decreases the weight of a data point if the data point is found within multiple clusters in the sample library, reference library, and/or control library.

In some embodiments of any of the aspects, the weighting parameter comprises a frequency weighting parameter. In some embodiments of any of the aspects, the weighting parameter comprises a trustworthiness weighting parameter. In some embodiments of any of the aspects, the weighting parameter comprises a frequency weighting parameter and a trustworthiness weighting parameter. In some embodiments of any of the aspects, the frequency weighting parameter is applied before the trustworthiness weighting parameter is applied. In some embodiments of any of the aspects, the trustworthiness weighting parameter is applied before the frequency weighting parameter is applied. In some embodiments of any of the aspects, the frequency weighting parameter and the trustworthiness weighting parameter are applied simultaneously. In some embodiments of any of the aspects, the frequency weighting parameter and/or the trustworthiness weighting parameter are applied iteratively.

In some embodiments of any of the aspects, the frequency weighting parameter is applied to the same data points, clusters, or bins as the trustworthiness weighting parameter. In some embodiments of any of the aspects, the frequency weighting parameter is applied to a first subset of data points, clusters, or bins, and the trustworthiness weighting parameter is applied to a second subset of data points, clusters, or bins, wherein the first subset and second subset can comprise shared data points, clusters, or bins. In some embodiments of any of the aspects, the frequency weighting parameter is applied to a first subset of data points, clusters, or bins, and the trustworthiness weighting parameter is applied to a second subset of data points, clusters, or bins, wherein the first subset and second subset do not comprise shared data points, clusters, or bins.

In some embodiments of any of the aspects, the detection method comprises a clustering process, a frequency weighting parameter, or a trustworthiness weighting parameter. In some embodiments of any of the aspects, the detection method comprises a clustering process and a frequency weighting parameter. In some embodiments of any of the aspects, the detection method comprises a clustering process and a trustworthiness weighting parameter. In some embodiments of any of the aspects, the detection method comprises a clustering process, a frequency weighting parameter, and a trustworthiness weighting parameter. In some embodiments of any of the aspects, the clustering process is applied before the frequency weighting parameter and/or the trustworthiness weighting parameter are applied. In some embodiments of any of the aspects, the clustering process is applied after the frequency weighting parameter and/or the trustworthiness weighting parameter are applied. In some embodiments of any of the aspects, the clustering process is applied between the frequency weighting parameter and/or the trustworthiness weighting parameter are applied (e.g., frequency, clustering, trustworthiness; trustworthiness, clustering, frequency). In some embodiments of any of the aspects, the clustering process, the frequency weighting parameter and/or the trustworthiness weighting parameter are applied simultaneously. In some embodiments of any of the aspects, the clustering process, the frequency weighting parameter and/or the trustworthiness weighting parameter are applied iteratively.

In some embodiments of any of the aspects, the clustering process, the frequency weighting parameter, and the trustworthiness weighting parameter are applied to the same data points, clusters, or bins. In some embodiments of any of the aspects, the clustering process, the frequency weighting parameter, and the trustworthiness weighting parameter are applied to different subsets of data points, clusters, or bins, wherein the subsets comprise shared data points, clusters, or bins. In some embodiments of any of the aspects, the clustering process, the frequency weighting parameter, and the trustworthiness weighting parameter are applied to different subsets of data points, clusters, or bins, wherein the subsets do not comprise shared data points, clusters, or bins.

In some embodiments of any of the aspects, a frequency weighting parameter increases the weight of a data point and/or cluster if the cluster containing the data point comprises additional other data points. As a non-limiting example, a frequency weighting parameter can increase the weight of a data point and/or cluster by at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 30%, at least 40%, at least 50%, at least 60%, at least 70%, at least 80%, at least 90%, or at least 100%. In some embodiments of any of the aspects, the frequency weighting parameter increases the weight of a data point and/or cluster if the cluster containing the data point comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 other or additional data points. In some embodiments of any of the aspects, the frequency weighting parameter can be applied to all data points. In some embodiments of any of the aspects, the frequency weighting parameter can be applied to a subset of data points. In some embodiments of any of the aspects, the frequency weighting parameter can be applied to all clusters or bins. In some embodiments of any of the aspects, the frequency weighting parameter can be applied to a subset of clusters or bins.

In some embodiments of any of the aspects, a trustworthiness weighting parameter decreases the weight of a data point and/or cluster. As a non-limiting example, a trustworthiness weighting parameter can decrease the weight of a data point and/or cluster by at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 30%, at least 40%, at least 50%, at least 60%, at least 70%, at least 80%, at least 90%, or at least 100%. In some embodiments of any of the aspects, a trustworthiness weighting parameter decreases the weight of a data point and/or cluster if the data point is found within multiple clusters in the sample library, a reference library, and/or a control library. As a non-limiting example, if the data point is found in a cluster in the sample library and a cluster in a control library, the trustworthiness weighting parameter can decrease the weight of the data point and/or cluster. As another non-limiting example, if the data point is found in a cluster in the sample library and a cluster in at least 2, at least 3, at least 4, or at least 5 reference libraries, the trustworthiness weighting parameter can decrease the weight of the data point and/or cluster. As another non-limiting, if the data point is found in a cluster in the sample library and a cluster in only one reference library (e.g., one known microbe species, one known microbe strain, one set of at least two known microbe species or strains, one known microbe component, or a set of at least two known microbe components), the trustworthiness weighting parameter does not decrease the weight of the data point and/or cluster, as that data point may be unique to that microbe or set of microbes.

In some embodiments of any of the aspects, a trustworthiness parameter can further comprise a cluster proportion comparison. As a non-limiting example, a first cluster from a first library (e.g., a sample library, a reference library, or a control library) is compared to a second cluster having the same average m/z value from a second library (e.g., a sample library, a reference library, or a control library). In some embodiments of any of the aspects, a first cluster from a sample library is compared to a second cluster having the same average m/z value from a reference library. The total number of data points (e.g., peaks) between the first cluster and second cluster is determined, and a cluster proportion can be calculated by determining the proportion of data points in each cluster. As a non-limiting example, if the first cluster contains 50 data points, and the second cluster contains 50 data points, the cluster proportion is 50:50. As another non-limiting example, if the first cluster contains 15 data points, and the second cluster contains 85 data points, the cluster proportion is 15:85. As another non-limiting example, if the first cluster contains 85 data points, and the second cluster contains 15 data points, the cluster proportion is 85:15.

In some embodiments of any of the aspects, a cluster proportion from 15:85 to 85:15 is considered “trustworthy”, i.e., the weight of the data point and/or cluster (e.g., in the sample library) is not decreased. In some embodiments of any of the aspects, a cluster proportion of approximately 15:85, 16:84, 17:83, 18:82, 19:81, 20:80, 21:79, 22:78, 23:77, 24:76, 25:75, 26:74, 27:73, 28:72, 29:71, 30:70, 31:69, 32:68, 33:67, 34:66, 35:65, 36:64, 37:63, 38:62, 39:61, 40:60, 41:59, 42:58, 43:57, 44:56, 45:55, 46:54, 47:53, 48:52, 49:51, 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15 is trustworthy, and the weight of the data point and/or cluster (e.g., in the sample library) is not decreased. In some embodiments of any of the aspects, a cluster proportion of approximately 1:99, 2:98, 3:97, 4:96, 5:95, 6:94, 7:93, 8:92, 9:91, 10:90, 11:89, 12:88, 13:87, 14:86, 86:14, 87:13, 88:12, 89:11, 90:10, 91:9, 92:8, 93:7, 94:6, 95:5, 96:4, 97:3, 98:2, 99:1 is not trustworthy, and the weight of the data point and/or cluster (e.g., in the sample library) is decreased.

In some embodiments of any of the aspects, a cluster proportion from 10:90 to 90:10, or from 20:80 to 80:20, or from 25:75 to 75:25, or from 30:70 to 70:30, or from 35:65 to 65:35, or from 40:60 to 60:40, or from 45:55 to 55:45, is considered “trustworthy”, and the weight of the data point and/or cluster (e.g., in the sample library) is not decreased. In some embodiments of any of the aspects, a trustworthiness parameter can be denoted by the symbol tau (t). In some embodiments of any of the aspects, the trustworthiness parameter can be a positive integer from 0 to 100, for example 0, 1, 2, 3, 4, 5, 6, 7, 8, 9, 10, 20, 30, 40, 50, 60, 70, 80, 90, or 100.

In some embodiments of any of the aspects, the detection method further comprises assigning a score (also referred to herein as “a probability estimate” or “assigned probability value” or “probability”) to the sample based on similarity with each reference library. As a non-limiting example, the score can be a value from 0 to 1 or a probability percentage from 0% to 100%. In some embodiments of any of the aspects, a score is determined between a sample library and each reference library, i.e., if there are 5 reference libraries corresponding to 5 different microbe species, microbe strains, or sets of at least two microbe species or strains, then 5 scores comparing the sample library to each reference library are generated. In some embodiments of any of the aspects, the scores comparing the sample library to each reference library are normalized (i.e., probabilities that sum to 1 or “percent frequency (%)”). In some embodiments of any of the aspects, the microbe or set of microbes in the sample is identified as belonging to or identifying with a specific reference library (i.e., identified as the same species or strain as the reference library) if the score is above a predetermined threshold. As a non-limiting example, the threshold can be at least at least 0.6, 0.8, at least 0.9, at least 0.95, at least 0.99, at least 0.995, or at least 0.999.

In some embodiments of any of the aspects, especially when there are few data points (e.g., peaks) in the sample library (e.g., fewer than 100 data points, fewer than 50 data points, fewer than 30 data points, fewer than 10 data points), the threshold can be a p-value adjusted threshold, i.e., a threshold that a probability estimate must satisfy in order to be deemed significant at a certain confidence level. As a non-limiting example, if there are n reference libraries, 1 of which is the correct identification of the unknown sample, and k clusters, then one can treat every cluster contribution to the correct library as a “success” and to all others as “failures”, leading to a binomial model with k trials and probability of success 1/n. If α is the significance level, then the quantile function of this binomial distribution with target probability (1-α), divided by n, returns the adjusted threshold that the probability estimate must satisfy. A detailed schematic of the detection method is summarized in FIG. 6 and FIG. 7A-7C.

In some embodiments of any of the aspects, the species of the pathogen is identified. Accordingly, in some embodiments of any of the aspects, the detection method further comprises identifying the species of the microbe (or set of microbes or the species comprising at least one microbe component) detected in the sample according to the data points analyzed and outputting said species on a display. Non-limiting examples of displays include a personal computer (e.g., of the patient or of the clinician), mobile device, tablet, head-mounted wearable computing device comprising a display, wrist-mounted wearable computing device comprising a display, and the like.

In some embodiments of any of the aspects, the strain of the pathogen is identified. Accordingly, in some embodiments of any of the aspects, the detection method further comprises identifying the strain of the microbe (or set of strains or the strain(s) comprising at least one microbe component) detected in the sample according to the data points analyzed and outputting said strain on a display. As strains are a more stringent classification than species, it is anticipated that the threshold values for strain identification can be more stringent or higher than threshold values for species identification. In some embodiments of any of the aspects, the detection method provides a “No Match” determination of a species or a strain, i.e., the sample library does not match any of the reference libraries.

In some embodiments of any of the aspects, the detection method further comprises determining whether at least one of the microbes (or microbe(s) comprising at least one microbe component) detected in the sample is sensitive or resistant to an antimicrobial therapeutic according to the data points analyzed and outputting said sensitivity on a display. In some embodiments of any of the aspects, the antimicrobial sensitivity of a sample library can be determined by analyzing the sample library's data points and/or clusters, and/or comparing the sample library to at least one reference library of known resistance to a specific antimicrobial and/or at least one reference library of known susceptibility to a specific antimicrobial. If the sample library is determined to be most similar to a reference library of a microbe (or set of microbes or microbe(s) comprising at least one microbe component) with known resistance to a specific antimicrobial, then it is determined that the microbe (or set of microbes or microbe(s) comprising at least one microbe component) in the sample library is resistant to that specific antimicrobial. If the sample library is determined to be most similar to the reference library of a microbe (or set of microbes or microbe(s) comprising at least one microbe component) with known susceptibility to a specific antimicrobial, then it is determined that the microbe (or set of microbes or microbe(s) comprising at least one microbe component) in the sample library is susceptible to that specific antimicrobial.

In some embodiments of any of the aspects, the method of analyzing the sample library for determination of antimicrobial resistance can comprise analyzing the entire spectrum, i.e., all data points or clusters of the sample library. In some embodiments of any of the aspects, the method of analyzing the sample library for determination of antimicrobial resistance can comprise analyzing a portion of the spectrum, e.g., specific data points or clusters that correspond to an antimicrobial resistance marker or to an antimicrobial susceptibility marker. In some embodiments of any of the aspects, the microbe (or set of microbes or microbe(s) comprising at least one microbe component) of the sample library is the same species or same strain as the microbe (or set of microbes or microbe(s) comprising at least one microbe component) of the most similar reference library. In some embodiments of any of the aspects, the microbe (or set of microbes or microbe(s) comprising at least one microbe component) of the sample library is a different species or a different strain as the microbe (or set of microbes or microbe(s) comprising at least one microbe component) of the most similar reference library, but comprising the same antimicrobial resistance marker or the same antimicrobial susceptibility marker, examples of which are described further herein.

In some embodiments of any of the aspects, the detection method further comprises assigning a patient (or a non-patient sample) to an infection category according to the data points analyzed and outputting the infection category on a display. In some embodiments of any of the aspects, at least two infection categories can be assigned, such as un-infected and infected. In some embodiments of any of the aspects, the infection category can correspond to the amount of microbe quantified in a sample library by the mass spectrometric method or quantified by another method (e.g., ELLecSA as described herein). As a non-limiting example, at least 3 infection categories can be assigned, including but not limited to un-infected, minimally infected, moderately infected, and/or highly infected. In some embodiments of any of the aspects, an infection category can correspond to a numerical scale (e.g., 1-10), with for example higher numbers corresponding to a greater quantity of microbes in the sample. In some embodiments of any of the aspects, an infection category can comprise a sepsis category, including but not limited to non-sepsis, sepsis, severe sepsis, and septic shock, wherein the sepsis category corresponds to the quantity of microbes in the sample, especially a blood sample. In some embodiments of any of the aspects, the sepsis category corresponds to sepsis classifications known in the art, including but not limited to non-sepsis, sepsis (e.g., infection+2 or more systemic inflammatory response syndrome (SIRS) criteria), severe sepsis (e.g., infection+1 or more organ dysfunctions), and septic shock (e.g., infection+1 or more hypoperfusion criteria). In some embodiments of any of the aspects, the SIRS criteria include but are not limited to: temperature >100.4° F. or <96.8° F., heart rate >90 beats/minute, respiratory rate >20 breaths/minute or saturation <90% on room air or PaCO2≤32 mm Hg or the use of mechanical ventilation, and white blood cell count ≥12,000 or ≤4,000 cells/μL or >10% bands. See e.g., Bone R. C. Toward an epidemiology and natural history of SIRS (systemic inflammatory response syndrome) J. Am. Med. Assoc. 1992, 268(24): 3452-3455; Singer M., Deutschman C. S., Seymour C. W. The third international consensus definitions for sepsis and septic shock (sepsis-3) J. Am. Med. Assoc. 2016, 315(8): 801-810; the contents of each of which are incorporated herein by reference in their entireties.

In some embodiments of any of the aspects, the mass spectrometric detection method comprises using stable (i.e., non-radioactive) isotope-labeled compounds, e.g., to enhance differentiation between signal and noise peaks. In some embodiments of any of the aspects, any stable isotope-labeled compound that has an m/z value within the analysis range, and does not overlap with m/z values in the experimental samples, can be used. Used herein, the term “stable isotope-labeled compounds” refers to small molecules, peptides, or proteins labeled with stable (nonradioactive) isotopes of hydrogen, carbon, nitrogen, oxygen, sulfur, chlorine, and/or bromine. In some embodiments of any of the aspects, the isotopically-labeled compounds comprise one or several hydrogens, carbons, nitrogens, oxygens, sulfurs, chlorines, and/or bromines substituted with the stable isotopes 2H, 13C, 15N, 17O-18O, 33S-36S, 35Cl, 37Cl, 79Br, and/or 81Br, such that the labeled compound has a resulting mass of known value greater than the unlabeled compound. In some embodiments of any of the aspects, these stable isotypes can be used to label microbe derived proteins or lipids which allows for very clear differentiation between noise and relevant signals.

In some embodiments of any of the aspects, the detection method comprises mass corrections, which are done by calibrating the mass spectrometer to known standards. In some embodiments of any of the aspects, mass correction can be performed in order to narrow the bin size during spectra analysis. In some embodiments of any of the aspects, the calibration generates mass corrections across a single spectrum, across several spectra, or across an entire mass spectrometric run, depending on experiment set-up. The mass corrections apply to all m/z values in the spectra, except for the m/z values associated with the known standards.

In some embodiments of any of the aspects, the known standards comprise a single species, or a mixture of species, of known masses. The standards that are used to generate mass corrections (i.e. calibrate the instrument or spectrum) and can be isotopes or non-isotopically labeled compounds, as long as the masses of the standards are known prior to mass spectrometric analysis and the m/z values are within the range of data acquisition. Calibration using internal or external standards does not require prior knowledge of the concentration, however, the concentration must be high enough for m/z signals to be detectable.

In some embodiments of any of the aspects, the calibration is performed internally or externally, as both types of calibration generate mass corrections. Internal calibration is performed by mixing the standards into each experimental sample before mass spectrometric analysis. The mass spectrometer is then programmed to calibrate each spectrum to the standard peak(s) within the experimental sample. In some embodiments of any of the aspects, an internal standard, such as a known concentration of a specific protein or peptide with known mass, allows for sample to sample mass corrections within a spectra and provides greater mass accuracy. Such internal standards allow for greater stringency in clustering and matching, as well as, provides a greater number of signals to match against. Internal standards also allow for a normalization of signal intensity, which can be used as an additional metric for identification. In some embodiments of any of the aspects, external calibration is performed by spotting (e.g., in the case of MALDI or SELDI-MS) or injecting (e.g., in the case of ESI or GC-MS) the standards separately from the experimental samples. External calibration performs a mass correction to one or more experimental samples. The number of spectra that apply to a single external calibration is dependent on experimental set-up.

In some embodiments of any of the aspects, the detection method comprises a less stringent approach to noise exclusion. In a less stringent approach to noise exclusion, peaks with lower signal-to-noise ratios (e.g., than in the method of Example 1) can be included in the mass libraries. A less stringent approach to noise exclusion thus allows for the inclusion of low abundance signals that add to the number of signals to match against. Including these lower signal peaks also allows for the inclusion of signals that are found in more than one library, which permits multi-infection detection.

Described herein are methods of producing a profile for a microbe, e.g., for the identification of at least one microbe or at least one microbe component in a sample. As used herein, a “profile” refers to a pattern of m/z data points (also referred to as signals or peaks) or m/z clusters that can be used to identify at least one microbe or at least one microbe component in a sample, and a profile can also be referred to herein as a fingerprint, a percent frequency representation, a mass spectrum, spectrum, a mass library, a library, a dataset, or m/z plot. In some embodiments of any of the aspects, the pattern of the profile can comprise the number of m/z peaks, the number of m/z clusters, the relative intensity of each m/z peak, and/or the relative intensity of each m/z cluster. In some embodiments of any of the aspects, the profile can be unique to a specific microbe, microbe component, set of at least two microbes, or set of at least two microbe components, and as such can be used to identify an unknown microbe in a sample. In some embodiments of any of the aspects, the profile comprises data points obtained or output from a mass spectrometric method. In some embodiments of any of the aspects, the profile comprises data points obtained or output from a mass spectrometric method and detected and/or analyzed by any of the methods described herein (e.g., clustering process, weighting parameter, frequency weighting parameter, trustworthiness parameter, or any combination thereof).

In some embodiments of any of the aspects, the results of the detection step comprise a profile, wherein said profile indicates the presence or absence of at least one microbe or microbe component. In some embodiments of any of the aspects, the profile is specific to at least one microbe, at least one microbe component, at least one set of microbes, or at least one set of microbe components. In some embodiments of any of the aspects, each profile comprises a set of m/z peaks clustered for a single molecular signal of interest or multiple molecular signals of interest. In some embodiments of any of the aspects, each profile comprises a set of m/z clusters. In some embodiments of any of the aspects, the profile for the specific microbe(s) or specific microbe component(s) does not include any of the set of data points associated with a control library. In some embodiments of any of the aspects, the profile for the specific microbe(s) or specific microbe component(s) does not include at least one data point associated with a control library. In some embodiments of any of the aspects, at least one background data point (i.e., noise) has been removed or reduced in the profile for the specific microbe(s) or microbe component(s), such as by comparison with at least one control library or with at least two reference libraries.

In some embodiments of any of the aspects, the profile of the specific microbe, specific microbe component, specific set of microbes, or specific set of microbe components is distinguishable from the profiles of other microbes or microbe components or sets thereof. In some embodiments of any of the aspects, the profile of the specific microbe, specific microbe component, specific set of microbes, or specific set of microbe components is unique compared to the profiles of other microbes or microbe components or sets thereof. In some embodiments of any of the aspects, the profile of the specific microbe, specific microbe component, specific set of microbes, or specific set of microbe components is at least 10%, at least 20%, at least 30%, at least 40%, at least 50%, at least 60%, at least 70%, at least 80%, at least 90%, or 100% different from the profiles of other microbes or microbe components or sets thereof.

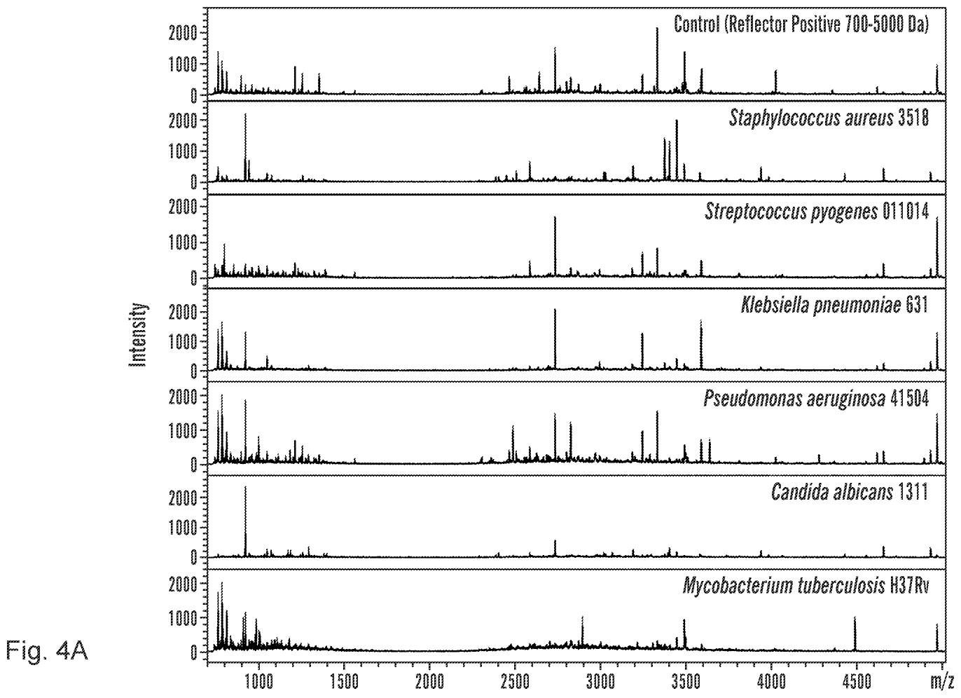

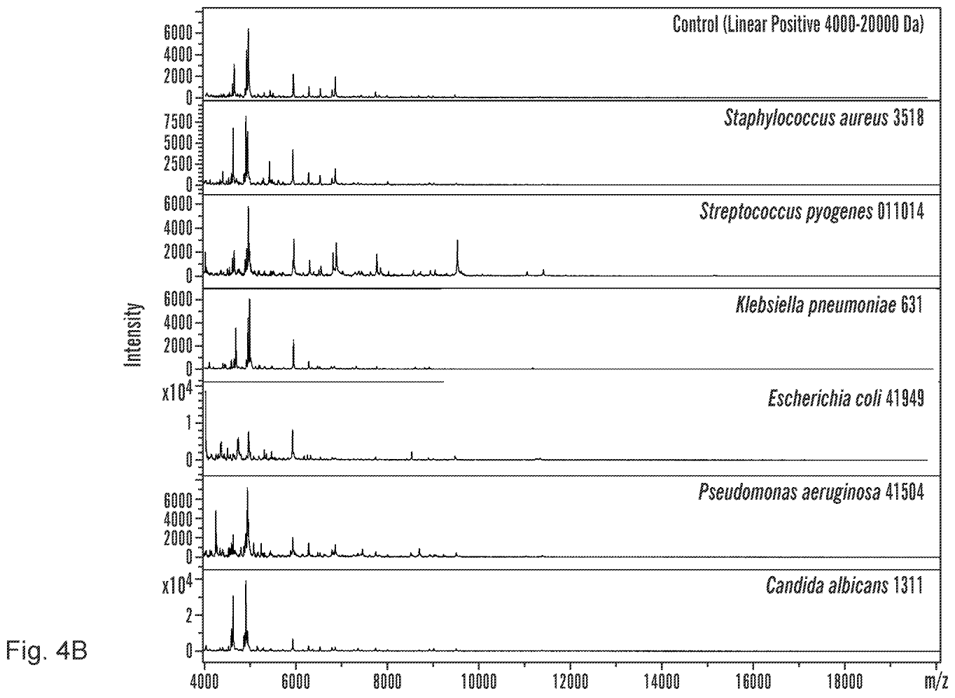

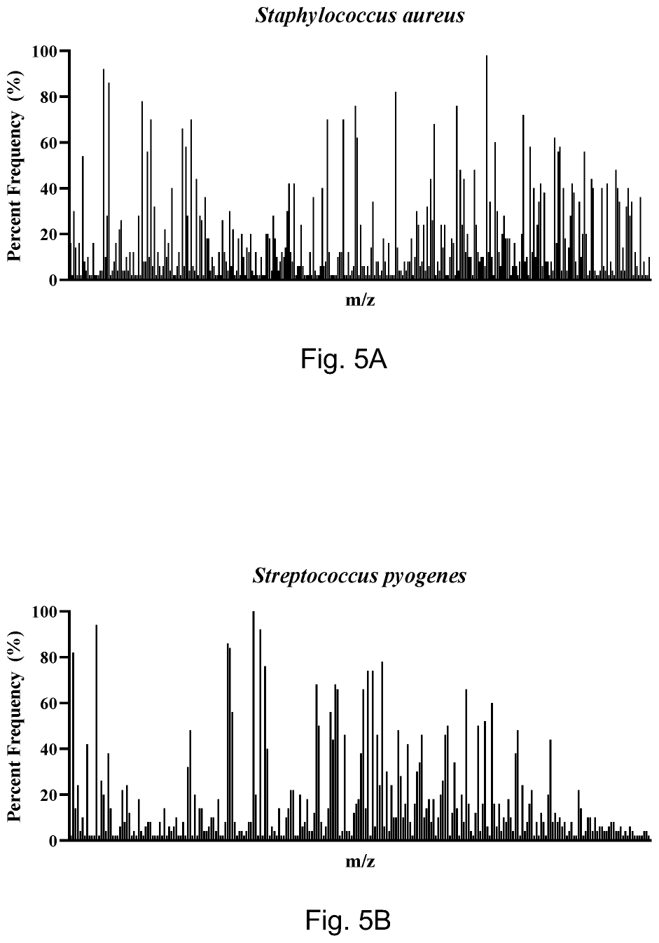

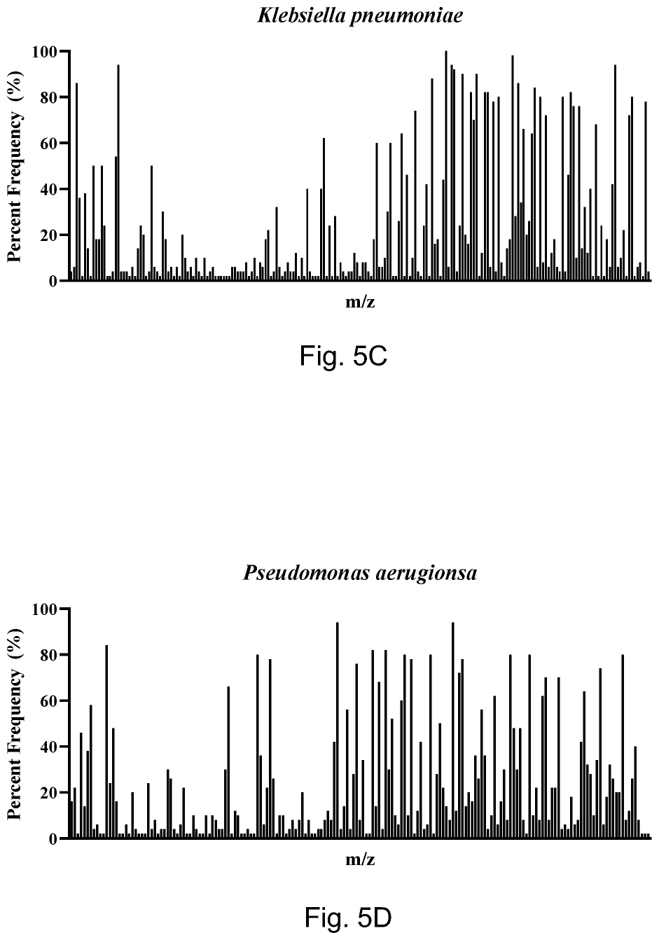

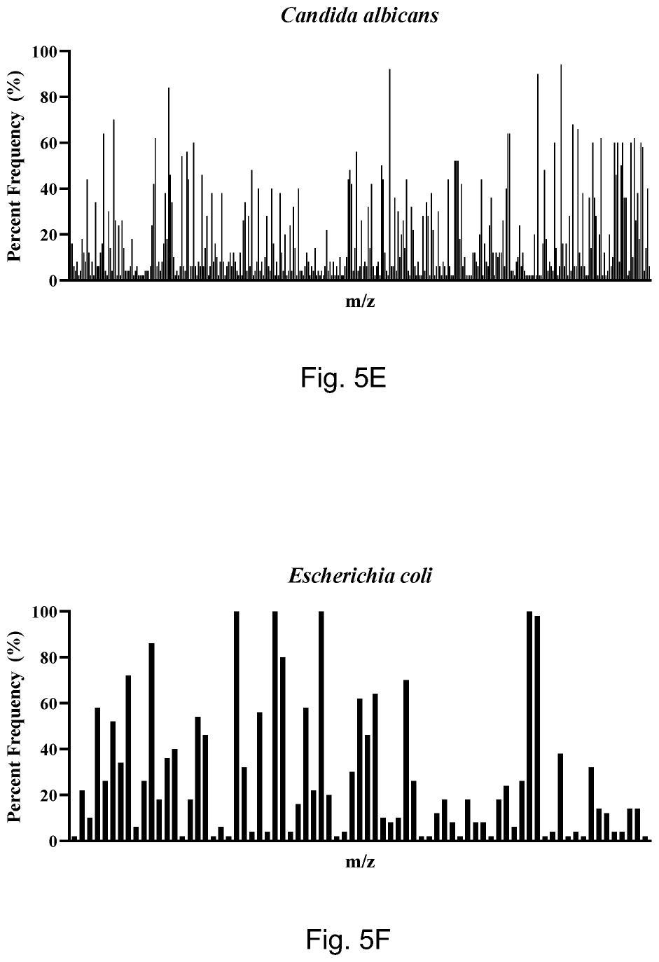

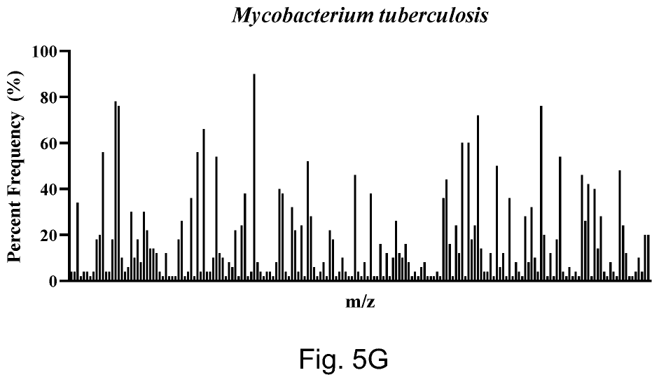

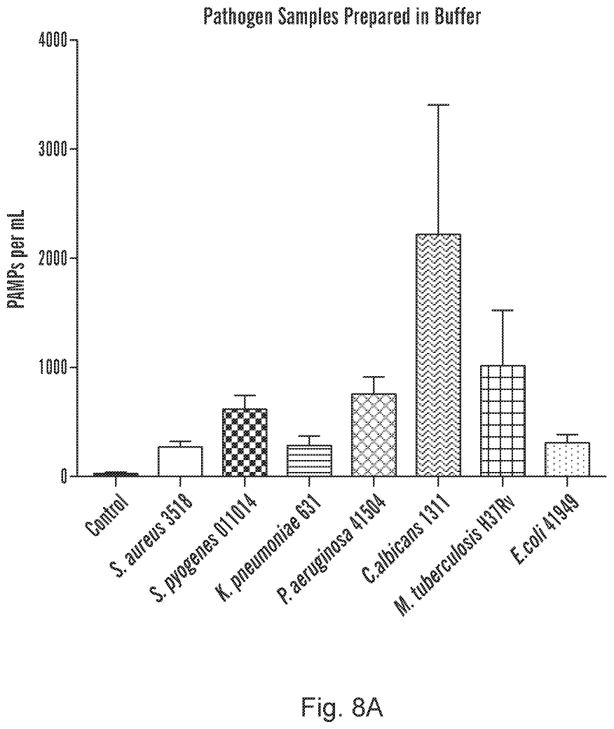

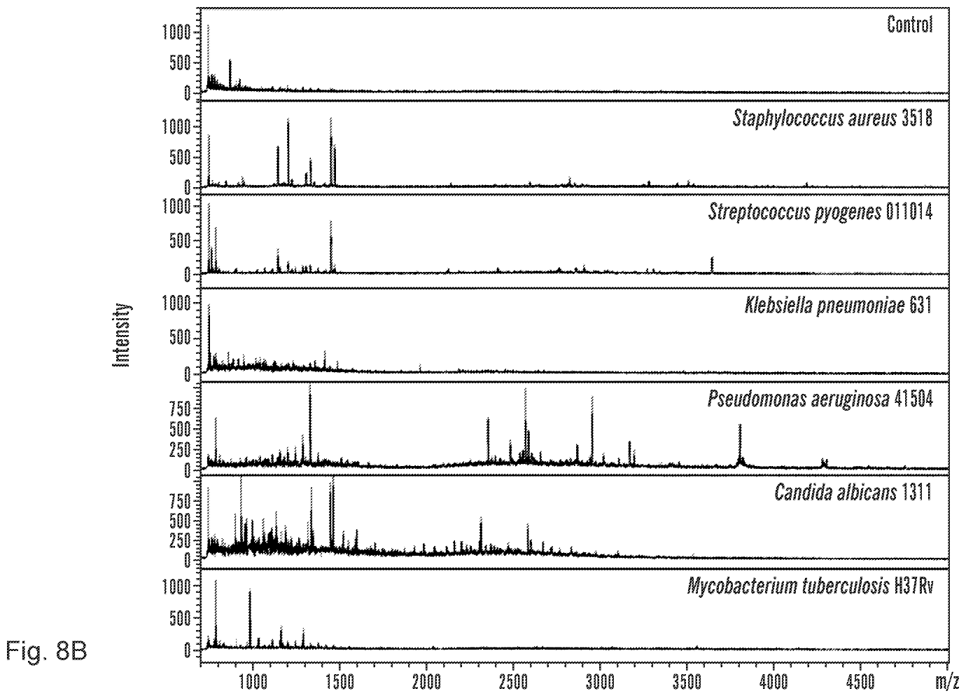

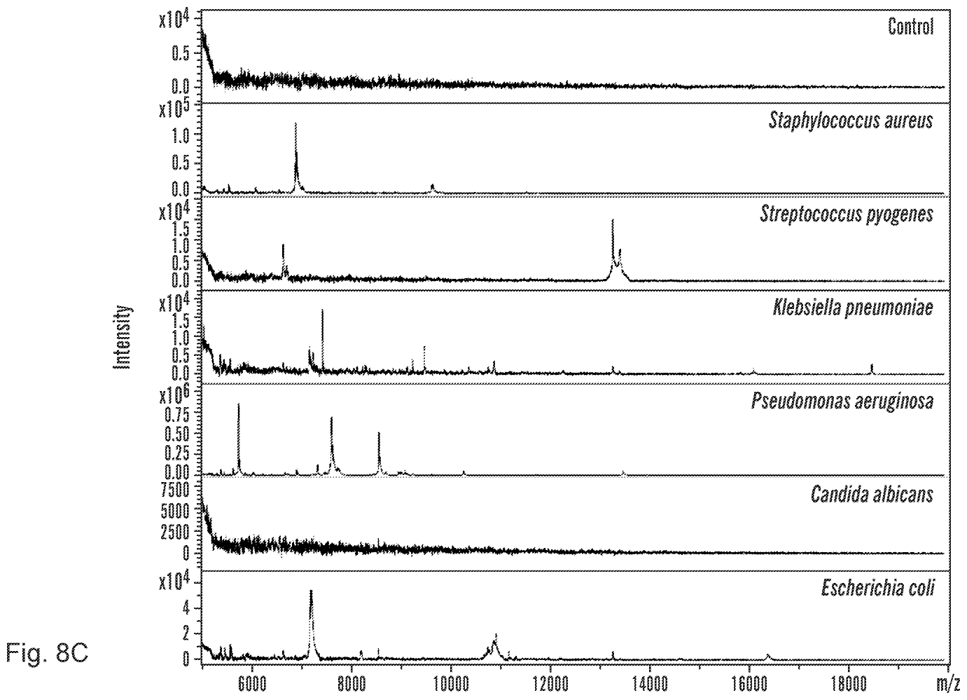

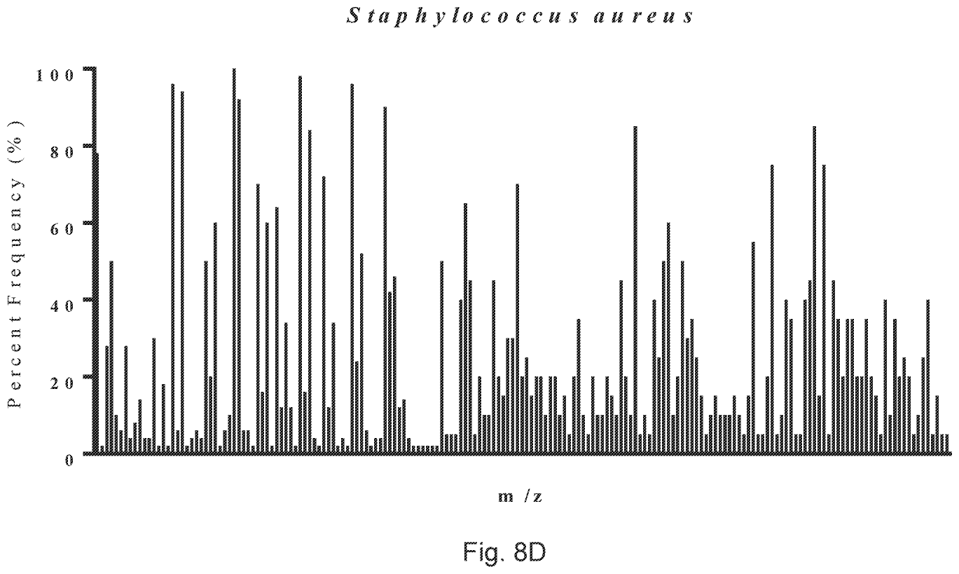

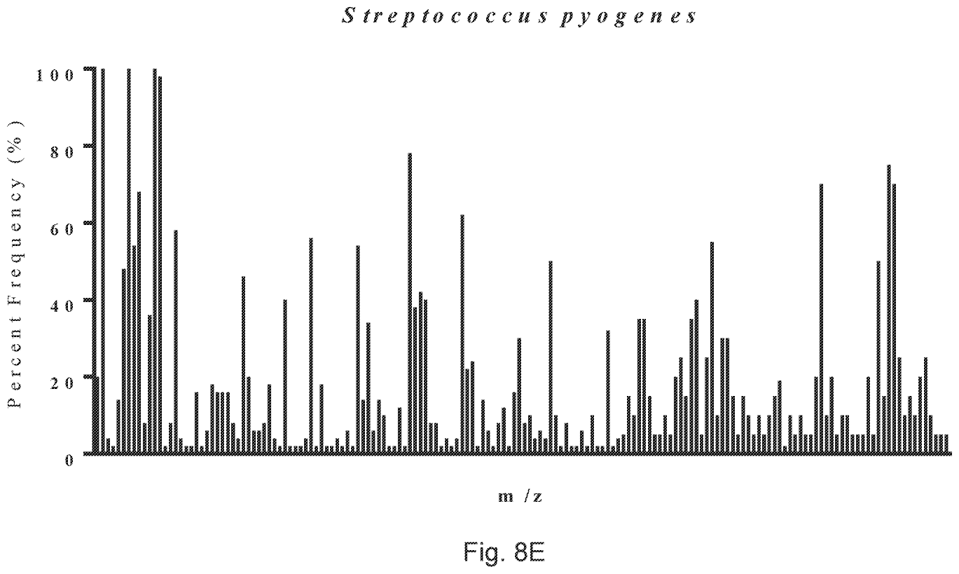

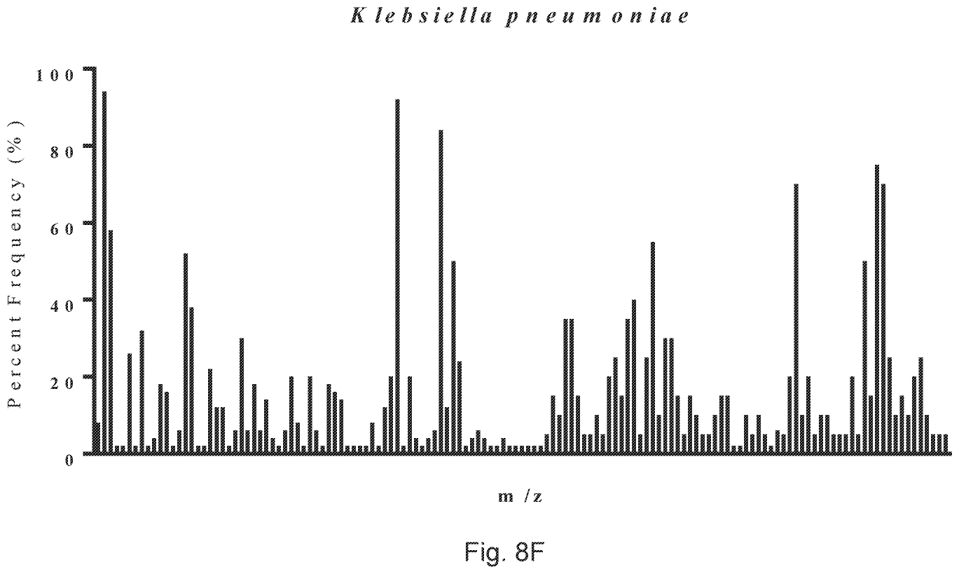

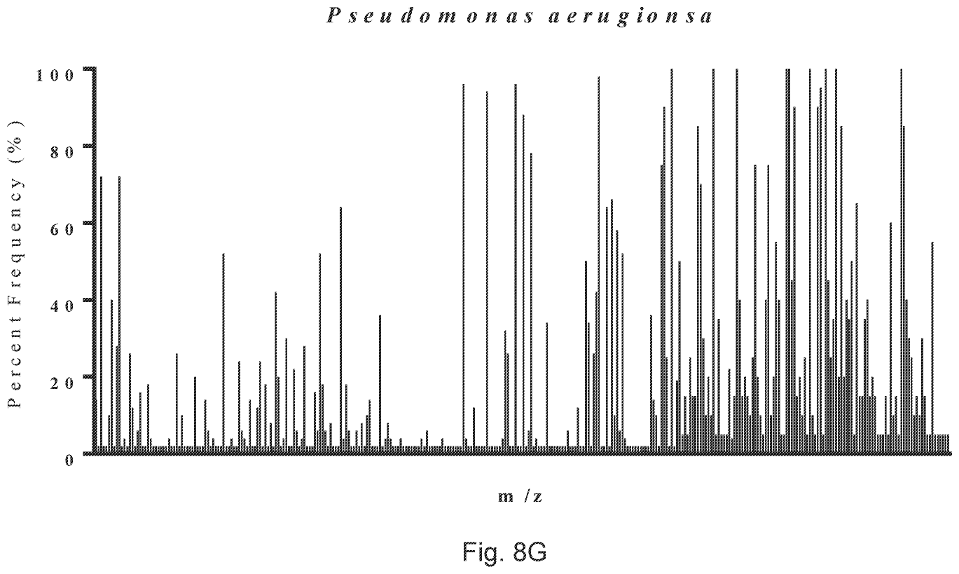

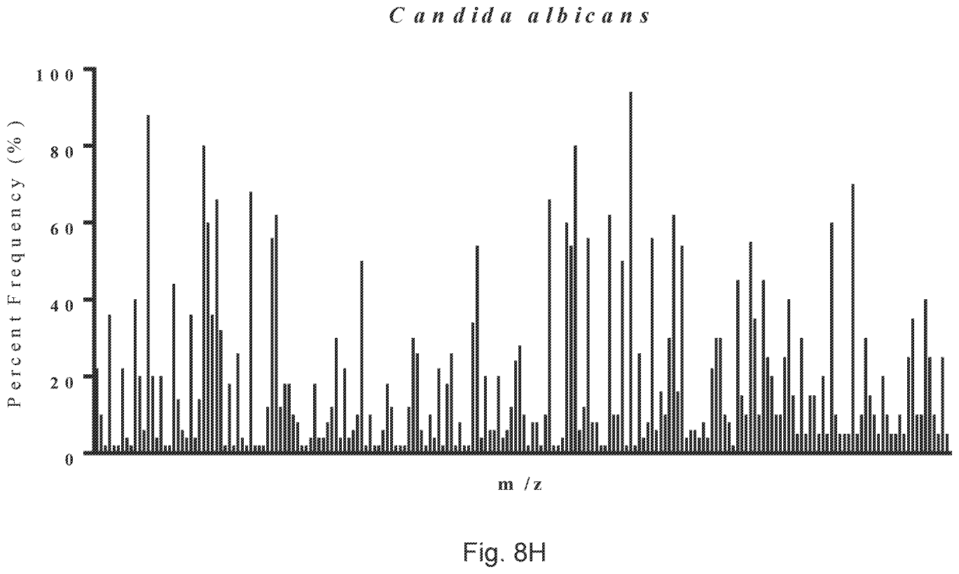

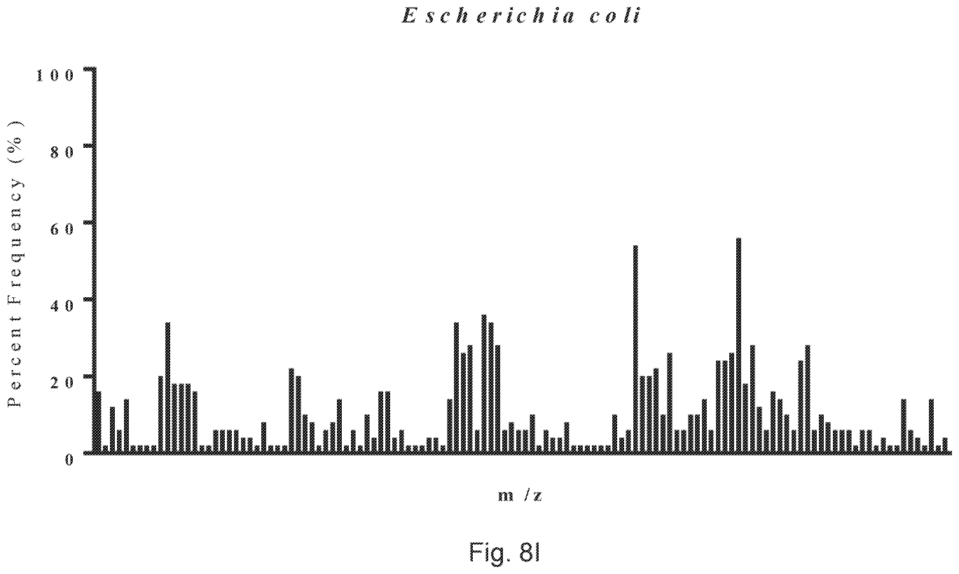

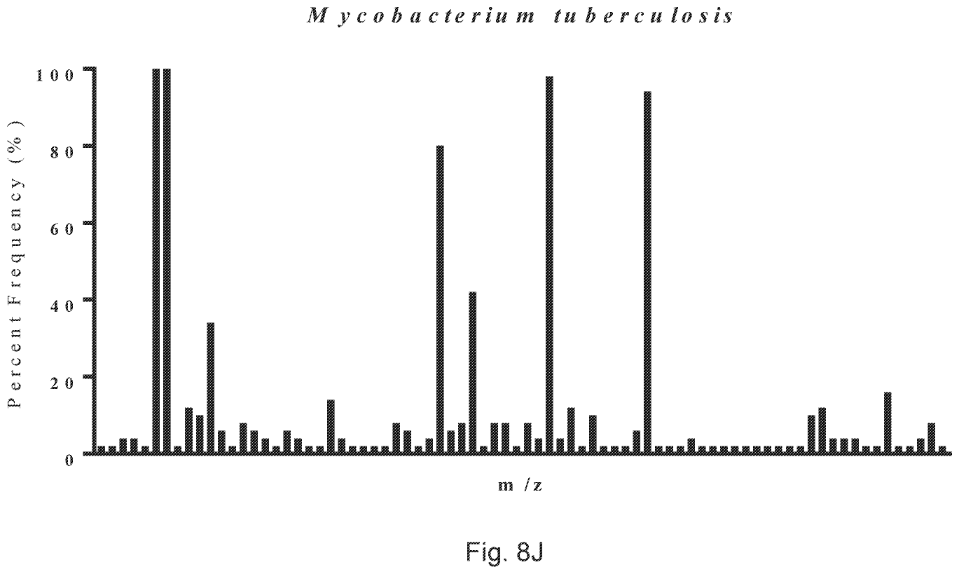

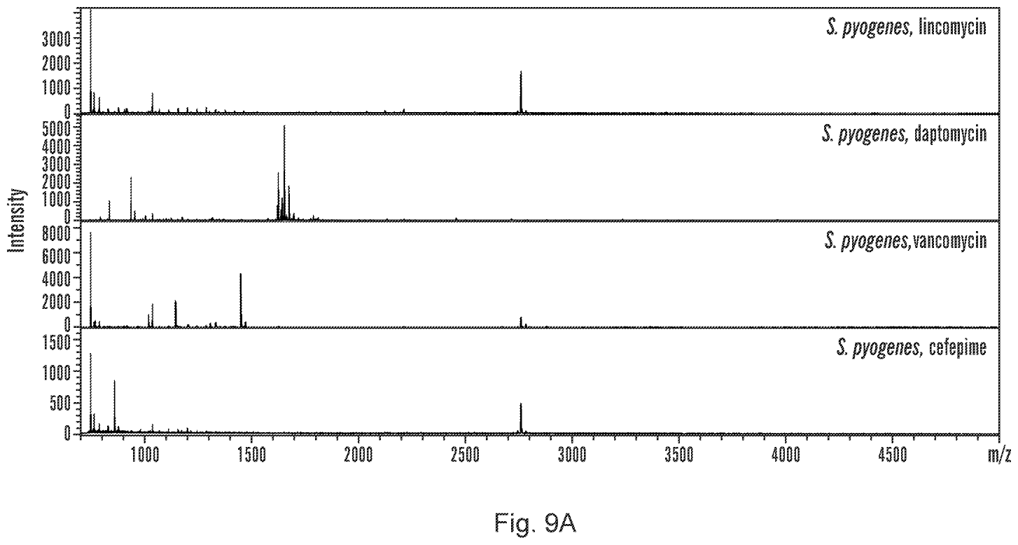

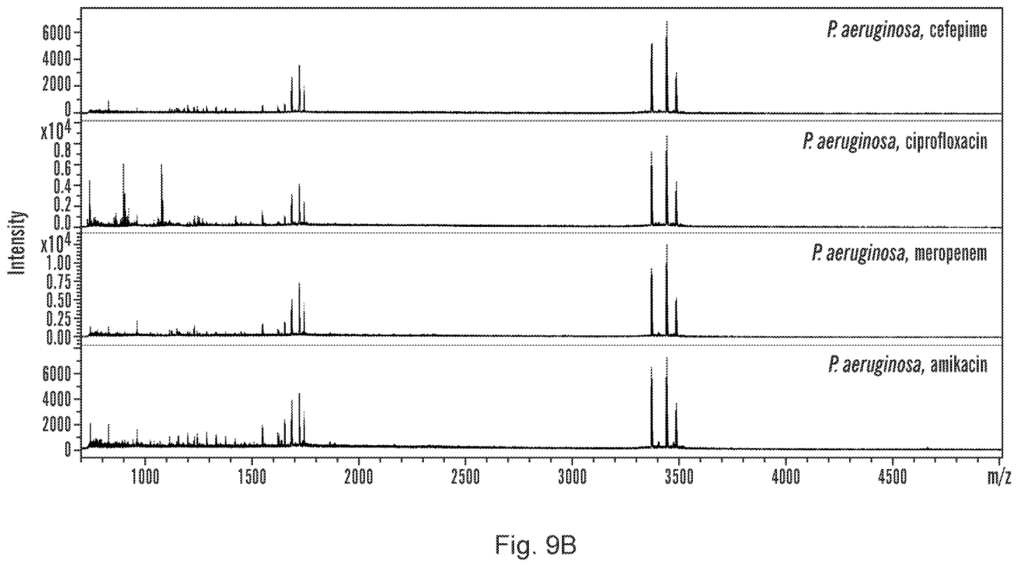

In some embodiments of any of the aspects, the profile is set forth in any one of FIG. 4A, FIG. 4B, FIG. 5A, FIG. 5A, FIG. 5B, FIG. 5C, FIG. 5D, FIG. 5E, FIG. 5F, FIG. 5G, FIG. 8B, FIG. 8C, FIG. 8D, FIG. 8E, FIG. 8F, FIG. 8G, FIG. 8H, FIG. 8I, FIG. 8J, FIG. 9A, or FIG. 9B. In some embodiments of any of the aspects, the control profile is set forth in any one of FIG. 4A, FIG. 4B, FIG. 8B, or FIG. 8C.

In some embodiments of any of the aspects, the profile for S. aureus (e.g., strain 3518) is set forth in any one of FIG. 4A, FIG. 4B, FIG. 5A, FIG. 8B, FIG. 8C, or FIG. 8D. In some embodiments of any of the aspects, the profile for S. aureus (e.g., strain 3518) comprises m/z peaks or m/z clusters at the following m/z values with the corresponding peak area in parentheses: 111.0596785 (43.99936659); 118.1443675 (41.82435722); 167.0517552 (716.874592); 168.0579552 (140.5539449); 183.0468352 (73.32261222); 211.2364329 (78.36644375); 327.0699069 (93.11972397); 332.0729119 (444.0931592); 345.0810222 (166.6396332); 398.085342 (108.12811); 433.077902 (131.1926651); 435.4216357 (113.5550754); 444.0939654 (93.10355558); 447.0893532 (120.3250169); 457.1022538 (109.0539002); 474.0812726 (163.6247488); 481.0984256 (126.5199578); 487.1080122 (139.6182017); 503.0847222 (140.2733929); 548.1144554 (157.1558937); 577.1191719 (184.1576559); 581.9713993 (66.51916557); 585.0939174 (159.482145); 586.1003147 (95.02974816); 587.1050808 (131.3909282); 593.1052243 (115.0980604); 603.1021645 (165.517285); 607.097126 (116.9388537); 612.0679377 (102.7730833); 619.0964037 (122.2015988); 621.0954841 (118.3744213); 623.1049898 (140.8231911); 644.1018695 (131.5184255); 647.0976507 (161.3976807); 655.0985385 (126.6552689); 669.0941179 (139.2066789); 671.0974467 (161.0861449); 675.0892865 (108.5968111); 728.0635875 (139.0653597); 739.113173 (138.5236286); 744.0382992 (121.7809869); 797.1045913 (134.9345892); and 799.0966511 (138.8447351).

In some embodiments of any of the aspects, the profile for S. pyogenes (e.g., strain 011014) is set forth in any one of FIG. 4A, FIG. 4B, FIG. 5B, FIG. 8B, FIG. 8C, FIG. 8E, or FIG. 9A.

In some embodiments of any of the aspects, the profile for K. pneumoniae (e.g., strain 631) is set forth in any one of FIG. 4A, FIG. 4B, FIG. 5C, FIG. 8B, FIG. 8C, or FIG. 8F.

In some embodiments of any of the aspects, the profile for E. coli (e.g., strain 41949) is set forth in any one of FIG. 4B, FIG. 5F, FIG. 8C, or FIG. 8I. In some embodiments of any of the aspects, the profile for E. coli (e.g., strain 41949) comprises m/z peaks or m/z clusters at the following m/z values with the corresponding peak area in parentheses: 342.08113 (75.21634636); 370.0090559 (71.35301324); 372.1790746 (121.0133317); 549.0146666 (185.9169218); 705.3316667 (72.08675852); 710.2016941 (156.7880747); 723.2111247 (116.8199995); and 892.0379653 (173.4197746).

In some embodiments of any of the aspects, the profile for P. aeruginosa (e.g., strain 41504) is set forth in any one of FIG. 4A, FIG. 4B, FIG. 5D, FIG. 8B, FIG. 8C, FIG. 8G, or FIG. 9B.

In some embodiments of any of the aspects, the profile for C. albicans (e.g., strain 1311) is set forth in any one of FIG. 4A, FIG. 4B, FIG. 5E, FIG. 8B, FIG. 8C, or FIG. 8H. In some embodiments of any of the aspects, the profile for C. albicans (e.g., strain 1311) comprises m/z peaks or m/z clusters at the following m/z values with the corresponding peak area in parentheses: 589.0722167 (107.5003545).

In some embodiments of any of the aspects, the profile forts. tuberculosis (e.g., strain H37Rv) is set forth in any one of FIG. 4A, FIG. 5G, FIG. 8B, or FIG. 8J.

TABLE 7
Representative m/z values for a fungus (C. albicans), a gram negative bacteria
(E. coli) and a gram positive bacteria (S. pneumoniae). Bold text represents the m/z
and area values are unique to the corresponding microbe. The samples
were processed as described herein (see e.g., Example 1).

| Fungus | | Gram-negative | | Gram-positive | |
|---|---|---|---|---|---|
| Candida albicans | | E. coli | | Streptococcus pneumoniae | |
| m/z | Area | m/z | Area | m/z | Area |
| 108.1049 | 41.98091 | 108.0966 | 97.37921 | 111.0597 | 43.99937 |
| 200.0192 | 299.3787 | 128.1176 | 65.21751 | 118.1444 | 41.82436 |
| 201.0208 | 60.13845 | 200.0157 | 375.9704 | 128.1246 | 96.00955 |
| 276.0799 | 316.5911 | 201.0176 | 77.34323 | 167.0518 | 716.8746 |
| 294.2616 | 104.3402 | 216.9872 | 72.05091 | 168.058 | 140.5539 |
| 332.0703 | 176.2372 | 231.0761 | 46.9108 | 183.0468 | 73.32261 |
| 336.2721 | 416.5758 | 238.1518 | 86.33124 | 200.025 | 341.3609 |
| 337.2743 | 92.84226 | 336.2712 | 544.1471 | 201.0253 | 64.32962 |
| 350.2493 | 140.0786 | 342.0811 | 75.21635 | 211.2364 | 78.36644 |
| 353.2687 | 64.59132 | 344.1451 | 63.30709 | 216.1809 | 65.23116 |
| 364.1585 | 53.92755 | 350.2496 | 164.795 | 231.0859 | 97.59611 |
| 410.0484 | 217.6118 | 364.1565 | 71.91542 | 327.0699 | 93.11972 |
| 423.0886 | 63.68621 | 370.0091 | 71.35301 | 332.0729 | 444.0932 |
| 589.0722 | 107.5004 | 372.1791 | 121.0133 | 336.2743 | 596.2555 |
| 712.0476 | 123.2043 | 378.0052 | 302.1733 | 344.0727 | 128.1848 |
| | | 382.0843 | 80.62543 | 345.081 | 166.6396 |
| | | 394.0825 | 115.9757 | 350.2624 | 196.0832 |
| | | 407.0582 | 116.8867 | 378.0221 | 158.233 |
| | | 423.085 | 126.6655 | 382.0909 | 128.1685 |
| | | 450.0681 | 133.6086 | 394.0921 | 134.7904 |
| | | 499.0513 | 132.9546 | 398.0853 | 108.1281 |
| | | 549.0147 | 185.9169 | 407.0637 | 157.373 |
| | | 555.032 | 105.2429 | 410.0523 | 520.266 |
| | | 560.0285 | 158.6682 | 423.0956 | 197.909 |
| | | 601.0799 | 140.8419 | 433.0779 | 131.1927 |
| | | 603.0895 | 135.8364 | 435.4216 | 113.5551 |
| | | 632.0888 | 103.9321 | 444.094 | 93.10356 |
| | | 639.0699 | 136.7507 | 447.0894 | 120.325 |
| | | 681.1682 | 186.0301 | 450.0829 | 179.7768 |
| | | 683.0811 | 120.3435 | 457.1023 | 109.0539 |
| | | 705.3317 | 72.08676 | 468.0892 | 126.5488 |
| | | 710.2017 | 156.7881 | 474.0813 | 163.6247 |
| | | 717.0235 | 189.7319 | 481.0984 | 126.52 |
| | | 723.2111 | 116.82 | 487.108 | 139.6182 |
| | | 797.0688 | 92.06294 | 499.0778 | 169.4498 |
| | | 877.0799 | 117.8066 | 503.0847 | 140.2734 |
| | | 892.038 | 173.4198 | 548.1145 | 157.1559 |
| | | 1052.033 | 151.8808 | 555.0727 | 142.3628 |
| | | | | 560.0604 | 182.5218 |
| | | | | 570.0988 | 95.39492 |
| | | | | 577.1192 | 184.1577 |
| | | | | 581.9714 | 66.51917 |
| | | | | 585.0939 | 159.4821 |
| | | | | 586.1003 | 95.02975 |
| | | | | 587.1051 | 131.3909 |
| | | | | 593.1052 | 115.0981 |
| | | | | 598.0941 | 122.8586 |
| | | | | 601.095 | 155.5786 |
| | | | | 603.1022 | 165.5173 |
| | | | | 607.0971 | 116.9389 |
| | | | | 612.0679 | 102.7731 |
| | | | | 619.0964 | 122.2016 |
| | | | | 621.0955 | 118.3744 |
| | | | | 623.105 | 140.8232 |
| | | | | 627.0989 | 139.651 |
| | | | | 632.1037 | 116.9498 |
| | | | | 639.1028 | 181.0785 |
| | | | | 644.1019 | 131.5184 |
| | | | | 647.0977 | 161.3977 |
| | | | | 655.0985 | 126.6553 |
| | | | | 669.0941 | 139.2067 |
| | | | | 671.0974 | 161.0861 |
| | | | | 675.0893 | 108.5968 |
| | | | | 681.1062 | 166.9497 |
| | | | | 683.0907 | 126.0661 |
| | | | | 712.0575 | 207.8939 |
| | | | | 728.0636 | 139.0654 |
| | | | | 739.1132 | 138.5236 |
| | | | | 744.0383 | 121.781 |
| | | | | 797.1046 | 134.9346 |
| | | | | 799.0967 | 138.8447 |
| | | | | 877.0864 | 141.2947 |
| | | | | 898.0668 | 135.4922 |
| | | | | 1052.079 | 141.1547 |

In some embodiments of any of the aspects, the profile comprises data points obtained from a mass spectrometric method comprising a positive reflector mode and/or data points obtained from a mass spectrometric method comprising a linear positive mode. As a non-limiting example, data points obtained from a reflector positive mode, comprise data points in the m/z range of 700 Da-5,000 Da. As a non-limiting example, data points obtained from a linear positive mode, comprise data points in the m/z range of 4,000 Da-20,000 Da. In some embodiments of any of the aspects, the profile comprises data points obtained from a mass spectrometric method comprising a positive reflector mode. In some embodiments of any of the aspects, the profile comprises data points obtained from a mass spectrometric method comprising a linear positive mode. In some embodiments of any of the aspects, the profile comprises data points obtained from a mass spectrometric method comprising a positive reflector mode and data points obtained from a mass spectrometric method comprising a linear positive mode. In some embodiments of any of the aspects, data points obtained from both a reflector positive mode and a linear positive mode can be combined using a method as described herein, e.g., a clustering process.

In some embodiments of any of the aspects, each profile comprises data obtained from at least one sample comprising at least one microbe or components of at least one microbe. In some embodiments of any of the aspects, the profile can comprise data obtained from at least 1, at least 2, at least 3, at least 4, at least 5, at least 6, at least 7, at least 8, at least 9, at least 10, at least 20, at least 30, at least 40, at least 50, or at least 100 samples of the same microbe species, the same microbe strain, the same set of at least two microbe species or strains, the same microbe component, or the same set of at least two microbe components. In some embodiments of any of the aspects, the profile can comprise samples from the same patient, different patients, the same non-patient sample, or different non-patient samples. In some embodiments of any of the aspects, the profile can comprise data obtained from the same sample run on a mass spectrometer and/or detected at the same or different time points. In some embodiments of any of the aspects, data obtained from multiple samples can be combined, summed, and/or averaged to yield one profile for each microbe, microbe strain, set of at least two microbe species or strains, microbe component, or set of at least two microbe components. In some embodiments of any of the aspects, at least two profiles can be obtained, corresponding to at least two different species of microbes, or at least two different strains of microbes, or at least different sets each comprising at least two microbe species or strains, or at least two different microbe components, or at two least different sets each comprising at least two microbe components. In some embodiments of any of the aspects, at least two profiles can be obtained, corresponding to the same species of microbes, the same strains of microbes, the same set of at least two microbe species or strains, the same microbe components, or the same set of at least two microbe components, but comprising different sampling and/or processing conditions. In some embodiments of any of the aspects, profiles can be obtained from least 2, at least 3, at least 4, at least 5, at least 6, at least 7, at least 8, at least 9, at least 10, at least 20, at least 30, at least 40, at least 50, or at least 100 microbe species, microbe strains, sets of at least two microbe species or strains, microbe components, or sets of at least two microbe components. In some embodiments of any of the aspects, a profile can refer to a sample profile, a reference profile, or a control profile. In some embodiments of any of the aspects, a profile can refer to a sample library, a reference library, or a control library, as described further herein.

Described herein are methods of detecting microbe or microbe components using a mass spectrometric method. In some embodiments of any of the aspects, the mass spectrometric method comprises Matrix-Assisted Laser Desorption Ionization (MALDI-TOF) mass spectrometry (MS). In some embodiments of any of the aspects, a MALDI-TOF machine, MALDI-TOF device, and/or MALDI-TOF system is used for detection of at least one microbe and/or at least one microbe component. MALDI-TOF can be preferable for the detection of microbes or microbe components as it creates ions from large molecules with minimal fragmentation. MALDI-TOF can be applied to the detection of biomolecules (e.g., biopolymers such as DNA, proteins, peptides and sugars) and large organic molecules (e.g., polymers, dendrimers and other macromolecules), which tend to be fragile and fragment when ionized by more conventional ionization methods. In some embodiments of any of the aspects, exemplary mass spectrometric methods (e.g., that can be paired with MALDI or any other mass spectrometric method as described herein) include, but are not limited to time of flight (TOF), quadrupole, triple quadrupole, high resolution and other mass spectrometric methods.

In embodiments in which MALDI cannot be used, additional exemplary ionization methods, prior to mass spectrometric detection, include but are not limited to liquid chromatography (LC), gas chromatography (GC), and electro-spray ionization (ESI). Accordingly, exemplary mass spectrometric methods include, but are not limited to MALDI-TOF, liquid chromatography MS (LC-MS), gas chromatography MS (GC-MS), electrospray ionization MS (ESI-MS), electron ionization MS, chemical ionization MS, atmospheric pressure chemical ionization MS, or surface-enhanced laser desorption/ionization (SELDI).

In some embodiments of any of the aspects, the mass spectrometric method is automated or at least partly automated. In some embodiments of any of the aspects, automated mass spectrometric method comprises an integrated model of automation joined by a conveyor system, automated processing of specimens, automated incubation, automated imaging of plates, automated reading of high-resolution plate images, automated discarding of plates when results are final, and/or automated delivery of plates to workbenches; see e.g., Theparee et al., J Clin Microbiol. 2018 January; 56(1): e01242-17.

In some embodiments of any of the aspects, the method of detecting microbe or microbe components comprises contacting the microbe or microbe components with a matrix or matrix solution on a target substrate. As used herein in regard to MALDI, the term “matrix” refers to a crystalline substrate that can be laser-absorbing, e.g., for the creation of ions from large molecules with minimal fragmentation. Examples of matrices are described further herein. As used herein, the term “matrix solution” refers to at least one matrix dissolved in a liquid substrate. In some embodiments of any of the aspects, the target substrate comprises a MALDI plate as known in the art. In some embodiments of any of the aspects, the MALDI plate can be metal. In some embodiments of any of the aspects, the MALDI plate comprises a highly regular fine structure on the plate surface, enabling highly homogenous co-crystallized preparations. In some embodiments of any of the aspects, the target substrate comprises locations for multiple samples, e.g., a multiplex MALDI plate.

In some embodiments of any of the aspects, the target substrate is evenly sprayed with matrix solution. In some embodiments of any of the aspects, the target substrate is evenly sprayed with matrix solution prior to contacting the isolated microbe or microbe components with the matrix, e.g., to generate a homogenous layer of crystallized matrix on top of the target substrate. In some embodiments of any of the aspects, the target substrate is evenly sprayed with matrix solution simultaneous to contacting the isolated microbe or microbe components with the matrix. In some embodiments of any of the aspects, the target substrate is evenly sprayed with matrix solution simultaneous to contacting the isolated microbe or microbe components with the matrix solution, i.e., the isolated microbe and microbe components are combined with the matrix solution and then evenly sprayed onto the target substrate.

In some embodiments of any of the aspects, the matrix solution comprises a matrix selected from the group consisting of 2,5-Dihydroxybenzoic acid (DHB), 2′,6′-dihydroxyacetophenone (DHAP), α-Cyano-4-hydroxycinnamic acid (CHCA), sinapic acid (SA), super DHB, 2′,4′,6′-trihydroxyacetophenone monohydrate (THAP), and 9-aminoacridine (9-AA). In some embodiments of any of the aspects, the matrix is dissolved in an organic, aqueous solution, and can be referred to as a matrix solution. In some embodiments of any of the aspects, an organic, aqueous solution can comprise water, methanol, formic acid, ethanol, isopropyl alcohol, butanol, lactic acid, acetic acid, citric acid, oxalic acid, uric acid, malic acid, and the like or any combination thereof. In preferred embodiments, the matrix solution is 40 mg/mL DHB in ˜50% methanol, ˜50% water, 0.1% formic acid.

In some embodiments of any of the aspects, the matrix solution comprises approximately 40 mg/mL DHB. As a non-limiting example, the matrix solution comprises at least 1 mg/mL DHB, at least 10 mg/mL DHB, at least 20 mg/mL DHB, at least 30 mg/mL DHB, at least 40 mg/mL DHB, at least 50 mg/mL DHB, at least 60 mg/mL DHB, at least 70 mg/mL DHB, at least 80 mg/mL DHB, at least 90 mg/mL DHB, or at least 100 mg/mL DHB. In some embodiments of any of the aspects, the matrix solution comprises approximately 50% methanol. As a non-limiting example, the matrix solution comprises at least 1%, at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, or at least 90% ethanol. In some embodiments of any of the aspects, the matrix solution comprises approximately 50% water. As a non-limiting example, the matrix solution comprises at least 1%, at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, or at least 90% water. In some embodiments of any of the aspects, the matrix solution comprises approximately 0.1% formic acid. As a non-limiting example, the matrix solution comprises at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, or at least 1.0% formic acid. In some embodiments of any of the aspects, the matrix solution does not comprise contaminants or comprises an undetectable level of contaminants. In some embodiments of any of the aspects, the protein profile of at least one microbe or microbe component in a sample can be detected using a mass spectrometric method as described herein.

In some embodiments of any of the aspects, the lipid profile of at least one microbe or microbe component in a sample can be detected using a mass spectrometric method as described herein. See e.g., Cox et al., Strain-level bacterial identification by CeO₂-catalyzed MALDI-TOF MS fatty acid analysis and comparison to commercial protein-based methods, Sci Rep. 2015 Jul. 20, 5:10470; Bolt et al., Automated High-Throughput Identification and Characterization of Clinically Important Bacteria and Fungi using Rapid Evaporative Ionization Mass Spectrometry, Anal Chem. 2016 Oct. 4, 88(19):9419-9426; Basu et al., Metal Oxide Laser Ionization Mass Spectrometry Imaging (MOLI MSI) Using Cerium(IV) Oxide, Anal Chem. 2019 May 21; 91(10):6800-6807; the contents of each of which are incorporated by reference herein in their entireties.

In some embodiments of any of the aspects, the matrix or matrix solution comprises a metal oxide, e.g., in order to catalyze fragmentation of lipids to produce fatty acids for analysis or detection in the sample. In some embodiments of any of the aspects, the matrix or matrix solution comprises cerium oxide (CeO₂) Methods of using CeO₂ in mass spectrometric methods (e.g., MALDI-TOF) are known in the art and can be referred to as metal oxide laser ionization (MOLI MS). In some embodiments of any of the aspects, the matrix solution comprises 100 mg of CeO₂ added to one mL of n-hexane (see e.g., Cox et al. 2015, supra). In some embodiments of any of the aspects, the matrix solution comprises CeO₂ prepared fresh in 2:1 CHCl₃:MeOH (v/v) at the following concentrations: 0 mg/mL, 5 mg/mL, 10 mg/mL, 25 mg/mL, 50 mg/mL, or 100 mg/mL (see e.g., Basu et al. 2019, supra). In some embodiments of any of the aspects, the lipids of the sample are extracted prior to detection using a mass spectrometric method as described herein. Methods of lipid extraction are known in the art and include but are not limited to methanol/chloroform phase separation. In some embodiments of any of the aspects, the lipid profile of at least one microbe or microbe component in a sample can be detected using rapid evaporative ionization mass spectrometry (REIMS) (see e.g., Bolt et al. 2016, supra).

In some embodiments of any of the aspects, e.g., after the step of contacting the isolated microbe or microbe components with a matrix, the microbe or microbe components are detected using a mass spectrometric method. In some embodiments of any of the aspects, MALDI-TOF detection comprises using a pulsed laser to irradiate the sample, triggering ablation and desorption of the sample and matrix material. In some embodiments of any of the aspects, MALDI-TOF detection comprises ionization of the analyte molecules by being protonated or deprotonated in a hot plume of ablated gases, which can then be accelerated into a mass spectrometer for time-of-flight detection and/or analysis.

In some embodiments of any of the aspects, detection methods as described herein can be used to identify at least one microbe in sample. For example, the detection and/or analysis of the material eluted from the PRR-coated beads (e.g., FcMBL-coated beads) can be identified to either a molecular level or a general pattern, which can be subsequently matched to a known database of profiles derived from previous isolates or patient samples. The construction of a profile database and the algorithms used to match a sample to a microbe or group of microbes can rely on scores determined according to the presence or absence of known or unknown characteristics of individual microbes or microbe classes. In some embodiments of any of the aspects, a mass spectrometric method and associated analysis methods as described herein can be used to quantify at least one microbe in sample. As a non-limiting example, analysis of the area under the MS profile curve can allow quantification of the microbial matter or MAMPs captured on the PRR-coated beads. See e.g., US Patent Publication 2016/0146810, which is incorporated herein by reference in its entirety.

In some aspects as described herein, microbe or microbe components are contacted and subsequently isolated with an engineered microbe-targeting molecule linked to a support. As used herein, “an engineered microbe-targeting molecule” refers to any one of the molecules described herein (or described in patents or patent application incorporated by reference) that can bind to and isolate microbes or microbe components. The terms “an engineered microbe-targeting molecule” and “a microbe-binding molecule” are used interchangeably herein.

In some embodiments of any of the aspects, the engineered microbe-targeting molecule comprises a microbe surface-binding domain, in other words a domain that binds to the surface (e.g., a surface molecule, a surface PRR) of a microbe.

In some embodiments of any of the aspects, the microbe surface-binding domain comprises a mannose-binding lectin (MBL). In some embodiments of any of the aspects, the microbe surface-binding domain comprises a human mannose-binding lectin (MBL); see e.g., SEQ ID NO: 1 (or SEQ ID NO: 15). In some embodiments of any of the aspects, the microbe surface-binding domain comprises a mannose-binding lectin (MBL) of a primate, mouse, rat, hamster, rabbit, or any other subject as described herein. In some embodiments of any of the aspects, the microbe surface-binding domain comprises a portion of a human mannose-binding lectin (MBL) see e.g., SEQ ID NO: 2-3.

In some embodiments of any of the aspects, the microbe surface-binding domain comprises a carbohydrate recognition domain (CRD) of MBL (see e.g., SEQ ID NO: 4). In some embodiments of any of the aspects, the CRD is linked to an immunoglobulin or fragment thereof. In some embodiments of any of the aspects, the CRD is linked to an Fc component of human IgG1 (FcMBL); see e.g., SEQ ID NO: 6-8 or SEQ ID NO: 16-17.

In some embodiments of any of the aspects, the engineered microbe-targeting molecule (also referred to as a microbe-binding molecule) comprises an MBL, a carbohydrate recognition domain of an MBL, or a genetically engineered version of MBL (FcMBL) as described in one of International Application No. WO 2011/090954, filed Jan. 19, 2011; U.S. Pat. Nos. 9,150,631; 9,593,160; the contents of each of which are incorporated herein by reference in their entireties. Amino acid sequences for MBL and engineered MBL include, but are not limited to:

(i) MBL full length (SEQ ID NO: 1):
MSLFPSLPLL LLSMVAASYS ETVTCEDAQK
TCPAVIACSS PGINGFPGKD GRDGTKGEKG EPGQGLRGLQ GPPGKLGPPG NPGPSGSPGP
KGQKGDPGKS PDGDSSLAAS ERKALQTEMA RIKKWLTFSL GKQVGNKFFL TNGEIMTFEK
VKALCVKFQA SVATPRNAAE NGAIQNLIKE EAFLGITDEK TEGQFVDLTG NRLTYTNWNE
GEPNNAGSDE DCVLLLKNGQ WNDVPCSTSH LAVCEFPI
(ii) MBL full length (SEQ ID NO: 15):
SLFPSLPLL LLSMVAASYS ETVTCEDAQK
TCPAVIACSS PGINGFPGKD GRDGTKGEKG EPGQGLRGLQ GPPGKLGPPG NPGPSGSPGP
KGQKGDPGKS PDGDSSLAAS ERKALQTEMA RIKKWLTFSL GKQVGNKFFL TNGEIMTFEK
VKALCVKFQA SVATPRNAAE NGAIQNLIKE EAFLGITDEK TEGQFVDLTG NRLTYTNWNE
GEPNNAGSDE DCVLLLKNGQ WNDVPCSTSH LAVCEFPI
(iii) MBL without the signal sequence (SEQ ID NO: 2):
ETVTCEDAQK TCPAVIACSS
PGINGFPGKD GRDGTKGEKG EPGQGLRGLQ GPPGKLGPPG NPGPSGSPGP KGQKGDPGKS
PDGDSSLAAS ERKALQTEMA RIKKWLTFSL GKQVGNKFFL TNGEIMTFEK VKALCVKFQA
SVATPRNAAE NGAIQNLIKE EAFLGITDEK TEGQFVDLTG NRLTYTNWNE GEPNNAGSDE
DCVLLLKNGQ WNDVPCSTSH LAVCEFPI
(iv) Truncated MBL (SEQ ID NO: 3):
AASERKALQT EMARIKKWLT
FSLGKQVGNK FFLINGEIMT FEKVKALCVK FQASVATPRN AAENGAIQNL IKEEAFLGIT
DEKTEGQFVD LTGNRLTYTN WNEGEPNNAG SDEDCVLLLK NGQWNDVPCS
TSHLAVCEFP I
(v) Carbohydrate recognition domain (CRD) of MBL (SEQ ID NO: 4):
VGNKFFLTNG
EIMTFEKVKA LCVKFQASVA TPRNAAENGA IQNLIKEEAF LGITDEKTEG QFVDLTGNRL
TYTNWNEGEP NNAGSDEDCV LLLKNGQWND VPCSTSHLAV CEFPI
(vi) Neck + Carbohydrate recognition domain of MBL (SEQ ID NO: 5):
PDGDSSLAAS ERKALQTEMA RIKKWLTFSL GKQVGNKFFL TNGEIMTFEK VKALCVKFQA
SVATPRNAAE NGAIQNLIKE EAFLGITDEK TEGQFVDLTG NRLTYTNWNE GEPNNAGSDE
DCVLLLKNGQ WNDVPCSTSH LAVCEFPI
(vii) FcMBL.81 (SEQ ID NO: 6):
EPKSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGAPDGDSSLAASERKAL
QTEMARIKKWLTFSLGKQVGNKFFLTNGEIMTFEKVKALCVKFQASVATPRNAAENGAIQN
LIKEEAFLGITDEKTEGQFVDLTGNRLTYTNWNEGEPNNAGSDEDCVLLLKNGQWNDVPCS
TSHLAVCEFPI
(viii) FcMBL.81 (SEQ ID NO: 16):
EPKSSDKTHT CPPCPAPELL GGPSVFLFPP
KPKDTLMISR TPEVTCVVVD VSHEDPEVKFNWYVDGVEVH NAKTKPREEQ
YNSTYRVVSV LTVLHQDWLN GKEYKCKVSN KALPAPIEKT ISKAKGQPRE PQVYTLPPSR
DELTKNQVSL TCLVKGFYPS DIAVEWESNG QPENNYKTTPPVLDSDGSFF LYSKLTVDKS
RWQQGNVFSC SVMHEALHNH YTQKSLSLSP GAPDGDSSLAASERKALQTE
MARIKKWLTF SLGKQVGNKF FLTNGEINITF EKVKALCVKF QASVATPRNA AENGAIQNLI
KEEAFLGITD EKTEGQFVDL TGNRLTYTNW NEGEPNNAGS DEDCVLLLKN
GQWNDVPCST SHLAVCEFPI
(ix) AKT-FcMBL (SEQ ID NO: 7):
AKTEPKSSDKTHT CPPCPAPELL GGPSVFLFPP
KPKDTLMISR TPEVTCVVVD VSHEDPEVKF NWYVDGVEVH NAKTKPREEQ
YNSTYRVVSV LTVLHQDWLN GKEYKCKVSN KALPAPIEKT ISKAKGQPRE PQVYTLPPSR
DELTKNQVSL TCLVKGFYPS DIAVEWESNG QPENNYKTTP PVLDSDGSFF LYSKLTVDKS
RWQQGNVFSC SVMHEALHNH YTQKSLSLSP GAPDGDSSLA ASERKALQTE
MARIKKWLTF SLGKQVGNKF FLINGEIMTF EKVKALCVKF QASVATPRNA AENGAIQNLI
KEEAFLGITD EKTEGQFVDL TGNRLTYTNW NEGEPNNAGS DEDCVLLLKN
GQWNDVPCST SHLAVCEFPI
(x) FcMBL.111 (SEQ ID NO: 8):
EPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGATSKQVGNKFFLTNGEI
MTFEKVKALCVKFQASVATPRNAAENGAIQNLIKEEAFLGITDEKTEGQFVDLTGNRLTYTN
WNEGEPNNAGSDEDCVLLLKNGQWNDVPCSTSHLAVCEFPI
(xi) FcMBL.111 (SEQ ID NO: 17):
EPKSSDKTHT CPPCPAPELL GGPSVFLFPP
KPKDTLMISR TPEVTCVVVD VSHEDPEVKF NWYVDGVEVH NAKTKPREEQ
YNSTYRVVSV LTVLHQDWLN GKEYKCKVSN KALPAPIEKT ISKAKGQPRE PQVYTLPPSR
DELTKNQVSL TCLVKGFYPS DIAVEWESNG QPENNYKTTP PVLDSDGSFF LYSKLTVDKS
RWQQGNVFSC SVMHEALHNH YTQKSLSLSP GATSKQVGNKF FLINGEEVITF
EKVKALCVKF QASVATPRNA AENGAIQNLI KEEAFLGITD EKTEGQFVDL TGNRLTYTNW
NEGEPNNAGS DEDCVLLLKN GQWNDVPCST SHLAVCEFPI

In some embodiments of any of the aspects, the engineered microbe-targeting comprises an amino acid sequence selected from SEQ ID NO: 1-SEQ ID NO: 8 or SEQ ID NO: 15-SEQ ID NO: 17, or any sequence that is at least 95% identical to any one of SEQ ID NO: 1-SEQ ID NO: 8 or SEQ ID NO: 15-SEQ ID NO: 17, e.g., that retains the microbe-targeting function. In some embodiments of any of the aspects, the engineered microbe-targeting comprises an amino acid sequence selected from SEQ ID NO: 1-SEQ ID NO: 8 or SEQ ID NO: 15-SEQ ID NO: 17, or any sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to any one of SEQ ID NO: 1-SEQ ID NO: 8 or SEQ ID NO: 15-SEQ ID NO: 17, e.g., that retains the microbe-targeting function.

In some embodiments where the linker comprises a Fc region or a fragment thereof, the Fc region or a fragment thereof can comprise at least one mutation, e.g., to modify the performance of the engineered microbe-targeting molecules. For example, in some embodiments, a half-life of the engineered microbe-targeting molecules described herein can be increased, e.g., by mutating an amino acid lysine (K) at the residue 232 of SEQ ID NO: 9 to alanine (A). Other mutations, e.g., located at the interface between the CH2 and CH3 domains shown in Hinton et al (2004) J Biol Chem. 279:6213-6216 and Vaccaro C. et al. (2005) Nat Biotechnol. 23: 1283-1288, can be also used to increase the half-life of the IgG1 and thus the engineered microbe-targeting molecules.

SEQ ID NO: 9
EPKSSDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP GA

The full-length amino acid sequence of carbohydrate recognition domain (CRD) of MBL is shown in SEQ ID NO: 4. The carbohydrate recognition domain of an engineered MBL described herein can have an amino acid sequence of about 10 to about 300 amino acid residues, or about 50 to about 160 amino acid residues. In some embodiments, the microbe surface-binding domain can have an amino acid sequence of at least about 5, at least about 10, at least about 15, at least about 20, at least about 30, at least about 40, at least about 50, at least about 60, at least about 70, at least about 80, at least about 90, at least about 100, at least about 150 amino acid residues or more. Accordingly, in some embodiments, the carbohydrate recognition domain of the engineered MBL molecule can comprise SEQ ID NO: 4. In some embodiments, the carbohydrate recognition domain of the engineered MBL molecule can comprise a fragment of SEQ ID NO: 4. Exemplary amino acid sequences of such fragments include, but are not limited to, ND (SEQ ID NO: 10), EXN (SEQ ID NO: 11: where X is any amino acid, e.g., P), NEGEPNNAGS (SEQ ID NO: 12) or a fragment thereof comprising EPN, GSDEDCVLL (SEQ ID NO: 13) or a fragment thereof comprising E, and LLLKNGQWNDVPCST (SEQ ID NO: 14) or a fragment thereof comprising ND. Modifications to such CRD fragments, e.g., by conservative substitution, are also within the scope described herein. In some embodiments, the MBL or a fragment thereof used in the microbe surface-binding domain of the engineered microbe-targeting molecules described herein can be a wild-type molecule or a recombinant molecule.

The exemplary sequences provided herein for the carbohydrate recognition domain of the engineered microbe-targeting molecules are not construed to be limiting. For example, while the exemplary sequences provided herein are derived from a human species, amino acid sequences of the same carbohydrate recognition domain in other species such as mice, rats, porcine, bovine, feline, and canine are known in the art and within the scope described herein.

In some embodiments of any of the aspects, the engineered microbe targeting molecule comprises a “pattern recognition receptor” (PRR), as described herein. In some embodiments of any of the aspects, described herein are PRR-based assays or PRR-based isolation methods. The term “pattern recognition receptor-based assay” or “PRR-based assay” refers to a method/assay and/or composition used to bind a microbe and/or microbial matter (e.g., MAMPs) comprising use of at least one or more PRRs, where the term “PRR” is defined herein. In some embodiments of any of the aspects, a PRR-based assay can refer to capture of a microbe and/or microbial matter (e.g., MAMPs) comprising use of at least one or more PRRs. In some embodiments of any of the aspects, a PRR-based assay can refer to use of at least one or more PRRs to provide a detectable signal in the presence of a microbe and/or microbial matter (e.g., MAMPs). In some embodiments of any of the aspects, a PRR-based assay can refer to use of at least one or more PRRs to capture a microbe and/or microbial matter (e.g., MAMPs) and also to provide a detectable signal in the presence of the microbe and/or microbial matter (e.g., MAMPs). In these embodiments, the same or different PRRs can be used in both the capture and signal detection steps.

In some embodiments of any of the aspects, a PRR-based assay (e.g., lectin based assay) can comprise use of at least one PRR such as lectin (e.g., a mannan binding lectin or molecule) bound to a solid substrate for capturing or isolating the microbe or microbial matter from the sample for subsequent detection. In some embodiments of any of the aspects, the PRR-based assay (e.g., lectin based assay) can comprise use of at least one PRR such as lectin (e.g., a mannan binding lectin or molecule) conjugated with a detectable label for detecting the microbe or microbial matter in the sample or isolated from the sample.

In some embodiments of any of the aspects, the PRR used in the PRR-based assay can comprise at least a portion of a pentraxin family protein, such as C-reactive protein (CRP). In some embodiments of any of the aspects, the CRP can be a recombinant CRP, such as CRP-Fc. CRP is described in, for example, U.S. Provisional patent application No. 61/917,705, filed Dec. 18, 2013, and US Patent Publication US 2016/0311877, the contents of each of which are incorporated herein by reference in their entireties.

In some embodiments of any of the aspects, the PRR(s) used in a PRR-based assay can comprise a lectin-based molecule. As used herein, the term “lectin-based molecule” refers to a molecule comprising a microbe-binding domain derived from at least a portion of lectin. The term “lectin” as used herein refers to any molecules including proteins, natural or genetically modified (e.g., recombinant), that interact specifically with saccharides (e.g., carbohydrates). The term “lectin” as used herein can also refer to lectins derived from any species, including, but not limited to, plants, animals, insects and microorganisms, having a desired carbohydrate binding specificity. Examples of plant lectins include, but are not limited to, the Leguminosae lectin family, such as ConA, soybean agglutinin, peanut lectin, lentil lectin, and Galanthus nivalis agglutinin (GNA) from the Galanthus (snowdrop) plant. Other examples of plant lectins are the Gramineae and Solanaceae families of lectins. Examples of animal lectins include, but are not limited to, any known lectin of the major groups S-type lectins, C-type lectins, P-type lectins, and I-type lectins, and galectins. In some embodiments of any of the aspects, the carbohydrate recognition domain can be derived from a C-type lectin, or a fragment thereof. C-type lectin can include any carbohydrate-binding protein that requires calcium for binding (e.g., MBL). In some embodiments of any of the aspects, the C-type lectin can include, but are not limited to, collectin, DC-SIGN, and fragments thereof. Without wishing to be bound by theory, DC-SIGN can generally bind various microbes by recognizing high-mannose-containing glycoproteins on their envelopes and/or function as a receptor for several viruses such as HIV and Hepatitis C.

Accordingly, in some embodiments of any of the aspects, the PRR-based assay is a lectin based assay. In some embodiments of any of the aspects, the lectin based assay generally relies on the capture of microbes/microbial components (e.g., MAMPs) from a sample using lectin molecules bound to a solid substrate (e.g., polymeric or magnetic particles or beads), followed by detection of the materials captured from the sample. Without wishing to be bound by a theory, particles (e.g., magnetic or polymeric particles or mesoporous particles, etc.) of different sizes can be used for capturing/detecting different microbes intact vs. disrupted/lysed material. For example, inventors have shown that particles of smaller size (e.g., 128 nm) have a higher efficiency for capturing intact microbes. On the other hand, particles of larger size (e.g., 1 μm) have a higher efficiency for capturing microbial disrupted/lysed materials, e.g., MAMPs.

Accordingly, in some embodiments of any of the aspects, particles of larger size can be better suited, e.g., for used for capturing and/or detecting the absence or presence of the microbial matter (e.g., MAMPs) and the particles of smaller size can be used for capture of intact bacteria, e.g., for further testing such as antibiotic susceptibility. Particles of other sizes and/or alternative chemistry can also be used, e.g., depending on the types of microbes/microbial matter to be captured/detected.

In some embodiments of any of the aspects, the lectin used in the lectin based assay is a mannose binding lectin (MBL). In some embodiments of any of the aspects, the lectin is a recombinant lectin such as FcMBL. FcMBL is a fusion protein comprising a carbohydrate recognition domain (CRD) of MBL and a portion of immunoglobulin. In some embodiments of any of the aspects, the FcMBL further comprises a neck region of MBL. In some embodiments of any of the aspects, the N-terminus of FcMBL can comprise an oligopeptide adapted to bind a solid substrate and orient the CRD of MBL away from the solid substrate surface. Various genetically engineered versions of MBL (e.g., FcMBL) are described in International Application Nos. WO 2011/090954, WO 2013/012924, and WO 2014/144325 as well as U.S. Pat. Nos. 9,150,631, 9,593,160, and 10,551,379, the contents of each of which are incorporated herein by reference in their entireties. Lectins and other mannan binding molecules are also described in, for example, U.S. patent application Ser. No. 13/574,191 (now U.S. Pat. No. 9,150,631); PCT application no. PCT/U.S.2011/021603, PCT/U.S.2012/047201, and PCT/U.S.2013/028409; and U.S. Provisional application No. 61/691,983 filed Aug. 22, 2012, U.S. Pat. No. 9,632,085; the contents of all of which are incorporated herein by reference in their entireties. Thus, in some embodiments of any of the aspects, the lectin based assay is an FcMBL based assay. As used herein, the terms “FcMBL based detection,” “FcMBL based assay,” “FcMBL based detection method,” and variants thereof refer to target molecule capture/detection methods and compositions comprising use of a FcMBL or variants thereof for capturing microbes and/or microbial matter (e.g., MAMPs) and/or providing a detectable signal in the presence of microbes and/or microbial matter (e.g., MAMPs).

Exemplary lectin (e.g., FcMBL) based microbe detection assays and compositions are described in, for example, PCT application no. PCT/U.S.2012/047201, no. PCT/U.S.2013/028409, and no. PCT/U.S. Ser. No. 14/28,683, filed Mar. 14, 2014, and U.S. Provisional application No. 61/691,983 filed Aug. 22, 2012, No. 61/788,570 filed Mar. 15, 2013, No. 61/772,436 filed Mar. 4, 2013, No. 61/772,360 filed Mar. 4, 2013, content of all of which are incorporated herein by reference in their entireties.

In some embodiments of any of the aspects, the engineered microbe-targeting molecule is linked to a support. In some embodiments of any of the aspects, the support comprises a solid substrate Examples of solid substrate can include, but are not limited to, beads or particles (including nanoparticles, microparticles, polymer microbeads, magnetic microbeads, mesoporous particles, and the like), filters, fibers, screens, mesh, tubes, hollow fibers, scaffolds, plates, channels, gold particles, magnetic materials, medical apparatuses (e.g., needles or catheters) or implants, dipsticks or test strips, filtration devices or membranes, hollow fiber cartridges, microfluidic devices, mixing elements (e.g., spiral mixers), extracorporeal devices, and other substrates commonly utilized in assay formats, and any combinations thereof. In some embodiments of any of the aspects, the solid substrate can be a magnetic particle or bead.

In some embodiments of any of the aspects, the support is a magnetic support. In some embodiments of any of the aspects, the magnetic support is a superparamagnetic support. In some embodiments of any of the aspects, the magnetic support comprises a magnetic bead, a superparamagnetic bead, or a magnetic microbead. Accordingly, in some embodiments of any of the aspect, the engineered microbe-targeting molecule linked to a magnetic support comprises FcMBL streptavidin linked to superparamagnetic beads.

In some embodiments of any of the aspects, the support is a nanoparticle. In some embodiments of any of the aspects, the support is a non-magnetic nanoparticle, such as a silica nanoparticle. In some embodiments of any of the aspects, the support is mesoporous, such as mesoporous versions of silica, alumina, carbon, niobium, tantalum, titanium, zirconium, cerium, or tin. A mesoporous material is a material containing pores with diameters between 2 nm and 50 nm, according to IUPAC nomenclature. For comparison, IUPAC defines microporous material as a material having pores smaller than 2 nm in diameter and macroporous material as a material having pores larger than 50 nm in diameter. In some embodiments of any of the aspects, the support is mesoporous silica, such as mesoporous silica nanoparticles.

As a non-limiting example, the mesoporous material or mesoporous nanoparticle (e.g., mesoporous silica) comprises pores that are about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 39 nm, about 40 nm, about 41 nm, about 42 nm, about 43 nm, about 44 nm, about 45 nm, about 46 nm, about 47 nm, about 48 nm, about 49 nm, or about 50 nm in diameter.

In some embodiments of any of the aspects, the nanoparticle (e.g., a mesoporous nanoparticle such as mesoporous silica) has a total diameter of 20 nm-200 nm. As a non-limiting example, the nanoparticle (e.g., mesoporous silica) has a total diameter of about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some embodiments of any of the aspects, the mesoporous silica is selected from the group consisting of: MCM-41 (Mobil Composition of Matter No. 41), MCM-48, MCM-50, SBA-15 (Santa Barbara Amorphous type material), SBA-11, SBA-12, SAB-16, KIT-5 (Korea Advanced Institute of Science and Technology), COK (Centre for Research Chemistry and Catalysis), TUD-1 (Technische Universiteit Delft), HMM-33 (Hiroshima Mesoporous Material-33), and FSM-16 (folded sheets of mesoporous materials). See e.g., Narayan et al., Mesoporous Silica Nanoparticles: A Comprehensive Review on Synthesis and Recent Advances, Pharmaceutics 2018 Aug. 6; 10(3):118, the contents of which are incorporated herein by reference in its entirety. In some embodiments of any of the aspects, the mesoporous silica comprises silica rods.

In some embodiments of any of the aspects, the engineered microbe-targeting molecule is conjugated or linked to a support using an activation agent. Without limitations, any process and/or reagent known in the art for conjugation activation can be used. Exemplary surface activation method or reagents include, but are not limited to, 1-Ethyl-3-[3-dimethylaminopropyl]carbodiimide hydrochloride (EDC or EDAC), hydroxybenzotriazole (HOBT), N-Hydroxysuccinimide (NHS), 2-(1H-7-Azabenzotriazol-1-yl)-1,1,3, 3-tetramethyl uronium hexafluorophosphate methanaminium (HATU), silanization, surface activation through plasma treatment, and the like. In some embodiments of any of the aspects, the engineered microbe-targeting molecule is conjugated or linked to a support using EDC and/or NHS.

Accordingly, in one aspect described herein is an engineered microbe-targeting molecule comprising a lectin CRD and a linker conjugated to a non-magnetic support. In another aspect described herein is an engineered microbe-targeting molecule comprising an MBL CRD and a Fc domain (e.g., FcMBL) conjugated (e.g., using EDC and NHS) to mesoporous silica particles.

In some embodiments of any of the aspects, the engineered microbe-targeting molecule is linked to an ELISA plate. Use of an ELISA plate can allow for multiplexing of samples. Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries. Different forms of ELISA are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In some embodiments of any of the aspects, a microbe-targeting molecule is coated and/or immobilized on the solid phase of multi-well plate, i.e., conjugated to a solid surface (usually a polystyrene micro titer plate, e.g., an “ELISA plate”). Immobilization can be either non-specific (e.g., by adsorption to the surface) or specific (e.g. where another molecule immobilized on the surface is used to capture the microbe-targeting molecule). In some embodiments of any of the aspects, a microbe-targeting molecule used in the ELISA plate comprises an engineered microbe-targeting molecules as described herein. In some embodiments of any of the aspects, a microbe-targeting molecule used in the ELISA plate comprises an antibody that specifically binds to a microbe or microbe component.

After the microbe-targeting molecule is immobilized, the sample is added, forming a complex with the microbe-targeting molecule. Between each step the plate is typically washed with a mild detergent solution to remove any molecules that are not specifically bound. After the final wash step, the plate is prepared for detection by a mass spectrometric method, as described herein. Such preparation can include but is not limited to eluting the microbe or microbe components, contacting the microbe or microbe components with a protease, contacting the microbe or microbe components with a solution that is more acidic than the microbe or microbe components, and/or contacting the microbe or microbe components with a matrix or matrix solution. Additional methods of preparing the isolated microbe or microbe components for detection by a mass spectrometric method can be performed as described herein.

In some embodiments of any of the aspects, preparation steps and/or mass spectrometric analysis/detection steps can be performed using a high-throughput method. Such high-throughput preparation steps and/or high-throughput mass spectrometric detection steps can be performed using the original ELISA plate, using a second ELISA plate, using subsequent ELISA plates, using another (e.g., non-ELISA) multi-well plate, using another multiplexed method as known in the art, or any combinations thereof. Such preparation steps and/or mass spectrometric detection steps can be performed using a low-throughput method, such as preparing the isolated microbe or microbe components in separate tubes or separate plates.

In some embodiments of any of the aspects, described herein are methods of isolating microbes or microbe components. In some embodiments of any of the aspects, the step of isolating comprises applying a magnet to the sample, for example to capture an engineered microbe targeting molecule linked to a magnetic support. In some embodiments of any of the aspects, the use of magnetic microparticles (e.g., superparamagnetic microparticles) can allow for easier washing and recovery of the microparticles, for automating the time of incubation with the sample, and also for working with whole blood with no interference from the erythrocytes. In some embodiments of any of the aspects, the magnet can be any magnetic material capable, a handheld magnet, a magnet formatted to a plate design such as a multi-well magnetic separator, a neodymium magnet tube rack, an automated magnet, and the like.

In some embodiments of any of the aspects, the step of isolating comprises centrifuging or otherwise separating a sample comprising an engineered microbe targeting molecule linked to a polymeric bead or support such a mesoporous support.

In some embodiments of any of the aspects, the methods described herein comprise contacting a sample with an engineered microbe-targeting molecule linked to a support and isolating the microbe or microbe components bound to the engineered microbe-targeting molecule. The support can be any support as described herein, including but not limited to beads or particles (including nanoparticles, microparticles, polymer microbeads, magnetic microbeads, mesoporous particles, and the like), filters, fibers, screens, mesh, tubes, hollow fibers, scaffolds, plates, channels, gold particles, magnetic materials, medical apparatuses (e.g., needles or catheters) or implants, dipsticks or test strips, filtration devices or membranes, hollow fiber cartridges, microfluidic devices, mixing elements (e.g., spiral mixers), extracorporeal devices, and other substrates commonly utilized in assay formats, and any combinations thereof.

In some embodiments of any of the aspects, the step of isolating comprises washing the support with a buffer to remove unbound cells or biomolecules. The buffer can be any buffer as described herein, including but not limited to tris-buffered-saline, phosphate buffer saline, water, HPLC grade H₂O, comprising octyl-β-D-glucopyranoside and/or calcium (TBSG Ca2+). In some embodiments of any of the aspects, the step of washing can be performed at least 1, at least 2, at least 3, at least 4, or at least 5 times.

In some embodiments of any of the aspects, the step of isolating further comprises eluting the microbe or microbe components from the support. In some embodiments of any of the aspects, the step of eluting comprises heating to a temperature of at least 70° C. or shaking at a speed of at least 950 rpm for no longer than 30 minutes. In some embodiments of any of the aspects, the step of eluting comprises heating to a temperature of at least 70° C. In some embodiments of any of the aspects, the step of eluting comprises shaking at a speed of at least 950 rpm for no longer than 30 minutes.

In some embodiments of any of the aspects, the step of eluting comprises heating to a temperature of at least 70° C. and shaking at a speed of at least 950 rpm for no longer than 30 minutes. In some embodiments of any of the aspects, the step of eluting comprises heating to a temperature of at least 60° C., at least 65° C., at least 70° C., at least 71° C., at least 72° C., at least 73° C., at least 74° C., at least 75° C., at least 80° C., at least 85° C., at least 90° C., or at least 95° C. In some embodiments of any of the aspects, the step of eluting comprises shaking at a speed of at least 800 rpm, at least 900 rpm, at least 910 rpm, at least 920 rpm, at least 930 rpm, at least 940 rpm, at least 950 rpm, at least 960 rpm, at least 970 rpm, at least 980 rpm, at least 990 rpm, or at least 1000 rpm. In some embodiments of any of the aspects, the step of eluting comprises heating and/or shaking for no longer than 30 minutes. In some embodiments of any of the aspects, the step of eluting comprises heating and/or shaking for at most 10 minutes, at most 20 minutes, at most 30 minutes, at most 40 minutes, at most 50 minutes, at most 60 minutes, at most 70 minutes, at most 80 minutes, at most 90 minutes, or at most 100 minutes.

In some embodiments of any of the aspects, the heating to a temperature of at least 70° C. is performed in calcium-free water, or in water or another buffer that is free or substantially free of calcium. In some embodiments of any of the aspects, the step of eluting comprises treatment with ethylenediaminetetraacetic acid (EDTA). In some embodiments of any of the aspects, the step of eluting comprises treatment with any calcium chelator as known in the art.

In some embodiments of any of the aspects, the step of isolating does not comprise eluting the microbe or microbe components from the support (e.g., a mesoporous material). In some embodiments of any of the aspects, the support (e.g., a mesoporous material) is compatible with the mass spectrometry method and does not need to be removed from the sample. In some embodiments of any of the aspects, the microbe or microbe components and the support (e.g., a mesoporous material) can be contacted with matrix or matrix solution.

In some embodiments of any of the aspects, the step of isolating comprises concentrating the microbe or microbe components into a smaller volume from a larger volume of the sample. In some embodiments of any of the aspects, the isolated volume is less than the volume of the sample.

While, in some embodiments of any of the aspects, microbes and/microbial matter (e.g., MAMPs) can be captured by PRR-coated solid substrates prior to detection, in other embodiments, microbes and/or microbial matter (e.g., MAMPs) can also be detected by PRR-coated detectable label as defined herein, e.g., PRR-coated fluorescent molecule, without prior capture. In these embodiments, the microbes and/or microbial matter (e.g., MAMPs) can be bound, mounted or blotted onto a solid surface, e.g., a tissue surface, and a membrane surface.

The microbes and/or microbial matter (e.g., MAMPs) bound to PRR-coated (e.g., lectin-coated) solid substrates (e.g., polymeric or magnetic particles or beads) or a solid surface can be detected by any methods known in the art or as described herein. Examples of detection methods can include, but are not limited to, spectrometry, electrochemical detection, polynucleotide detection, fluorescence anisotropy, fluorescence resonance energy transfer, electron transfer, enzyme assay, magnetism, electrical conductivity, isoelectric focusing, chromatography, immunoprecipitation, immunoseparation, aptamer binding, filtration, electrophoresis, use of a CCD camera, immunoassay, ELISA, Gram staining, immunostaining, microscopy, immunofluorescence, western blot, polymerase chain reaction (PCR), RT-PCR, fluorescence in situ hybridization, sequencing, mass spectrometry, or substantially any combination thereof. The captured microbe can remain bound on the PRR-coated solid substrates during detection and/or analysis, or be isolated form the PRR-coated solid substrates prior to detection and/or analysis.

In some embodiments of any of the aspects, the microbes and/or microbial matter (e.g., MAMPs) bound to PRR-coated (e.g., lectin-coated) solid substrates (e.g., polymeric or magnetic particles or beads) can be detected by ELLecSA as defined herein, an example which is described in detail in the section “An exemplary enzyme-linked lectin sorbent assay (ELLecSA)” below. Additional information various embodiments of FcMBL based assays can be found, e.g., in PCT application no. PCT/U.S.2012/047201, no. PCT/U.S.2013/028409, and no. PCT/U.S. Ser. No. 14/28,683, the contents of all of which are incorporated herein by reference in their entireties.

In some embodiments of any of the aspects, compositions (e.g., engineered microbe targeting molecules as described further therein), methods, systems, and assays are further described in at least one of the following: U.S. Provisional Applications 61/296,222, 61/508,957, 61/604,878, 61/605,052, 61/605,081, 61/788,570, 61/846,438, 61/866,843, 61/917,705, 62/201,745, 62/336,940, 62/543,614; PCT application numbers PCT/US2011/021603, PCT/US2012/047201, PCT/US2013/028409, PCT/US2014/028683, PCT/US2014/046716, PCT/US2014/071293, PCT/US2016/045509, PCT/US2017/032928; U.S. patent application Ser. Nos. 13/574,191, 14/233,553, 14/382,043, 14/766,575, 14/831,480, 14/904,583, 15/105,298, 15/415,352, 15/483,216, 15/668,794, 15/750,788, 15/839,352, 16/059,799, 16/302,023, 16/553,635; and U.S. Pat. Nos. 9,150,631, 9,593,160, 9,632,085, 9,791,440, and 10,435,457; the contents of each of which are incorporated by reference herein in their entireties.

In some embodiments of any of the aspects, a microbe or microbe component can be isolated from a sample using methods described herein. The isolated microbe or microbe component can be prepared for detection, e.g., with MALDI-TOF MS. In some embodiments of any of the aspects, the isolated microbe or microbe components are digested with a protease. Digestion with a protease after isolation can standardize the isolated microbe or microbe components and can increase the probability of correctly identifying the microbe (see e.g., Example 2, Table 6).

In some embodiments of any of the aspects, the protease is selected from the group consisting of trypsin, chymotrypsin, pepsin, papain, elastase, or any combination thereof. The protease can also be any protease or protease cocktail known in the art. Non-limiting examples of proteases include serine proteases, cysteine proteases, threonine proteases, aspartic proteases, glutamic proteases, metalloproteases, asparagine peptide lyases. In some embodiments of any of the aspects, the isolated microbe or microbe components are digested with at least one protease, at least 2 proteases, at least 3 proteases, at least 4 proteases, or at least 5 proteases, concurrently and/or sequentially. In some embodiments of any of the aspects, the protease is substantially free of protease inhibitors (e.g., 4-(2-Aminoethyl)benzenesulfonyl fluoride hydrochloride (AEBSF), Aprotinin, Bestatin, E64, Leupeptin, Pepstatin A).

In some embodiments of any of the aspects, the protease is trypsin. It is noted that trypsin is not commonly used in MALDI-TOF detection of microbes. The most commonly used method of MALDI-TOF detection of microbes is the Biotyper™ method, which identifies ribosomal proteins; the Biotyper™ method uses cultured microbes applied directly to the target plate and layered with matrix before analysis by MALDI-TOF. Thus, digesting at least one microbe or microbe component with a protease (e.g., trypsin) is not a necessary step used to identify microbes using the most commonly used method of MALDI-TOF. In some embodiments of any of the aspects, the trypsin can be α-trypsin, β-trypsin, trypsin 1, trypsin 2, or mesotrypsin. In some embodiments of any of the aspects, the trypsin is at least 10% trypsin. As a non-limiting example, the trypsin is at least 1%, at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, or at least 90% trypsin. In some embodiments of any of the aspects, the trypsin is substantially free of trypsin inhibitors (e.g., Ca²⁺, Mg²⁺, heat, serpin, etc.). In some embodiments of any of the aspects, the trypsin comprises a divalent cation chelator (e.g., EDTA).

In some embodiments of any of the aspects, the isolated microbe or microbe component is digested for at most 30 seconds, at most 1 minute, at most 2 minutes, at most 3 minutes, at most 4 minutes, at most 5 minutes, at most 6 minutes, at most 7 minutes, at most 8 minutes, at most 9 minutes, at most 10 minutes, at most 20 minutes, at most 30 minutes, at most 40 minutes, at most 50 minutes, at most 60 minutes, at most 70 minutes, at most 80 minutes, at most 90 minutes, at most 2 hours, at most 3 hours, at most 4 hours, at most 5 hours, at most 6 hours, at most 7 hours, at most 8 hours, at most 9 hours, at most 10 hours, at most 11 hours, or at most 12 hours. In some embodiments of any of the aspects, the isolated microbe or microbe component is digested overnight.

In some embodiments of any of the aspects, the isolated microbe or microbe component is digested at human body temperature (e.g., 36-38° C.). In some embodiments of any of the aspects, the isolated microbe or microbe component is digested at a temperature that is greater than 36-38° C. In some embodiments of any of the aspects, the digestion of the isolated microbe or microbe component further comprises heating the digestion. Heating the protease can permit faster digestion and can increase the probability of correctly identifying the microbe (see e.g., Example 2, Table 6).

In some embodiments of any of the aspects, heating the digestion comprises microwave treatment. In some embodiments of any of the aspects, the microwave treatment of the digestion is at a power of least 500 watts (W), at least 600 W, at least 700 W, at least 800 W, at least 900 W, at least 1000 W, at least 1100 W, at least 1200 W, at least 1300 W, at least 1400 W, or at least 1500 W. In some embodiments of any of the aspects, the microwave treatment of the digestion occurs for 1 minute. As a non-limiting example, the microwave treatment of the digestion can occur for at most 10 seconds, at most 20 seconds, at most 30 seconds, at most 40 seconds, at most 50 seconds, at most 1 minute, at most 2 minutes, at most 3 minutes, at most 4 minutes, at most 5 minutes, at most 6 minutes, at most 7 minutes, at most 8 minutes, at most 9 minutes, or at most 10 minutes.

In some embodiments of any of the aspects, the method described herein further comprises contacting the digested microbe or microbe components with a composition that is more acidic than the digested microbe or microbe components (e.g., said step of contacting can decrease the pH of the solution). As used herein, “more acidic” refers to a composition or solution with a lower pH compared to another composition or solution. Contacting the digested microbe or microbe components with such a composition can quickly and effectively quench the protease digestion reaction, increase component stability, and improve mass spectrometry (e.g., MALDI-TOF) sensitivity.

In some embodiments of any of the aspects, the composition that is more acidic than the digested microbe or microbe components is present at a volume equal to or greater than the volume of the digested microbe or microbe components. As a non-limiting example, the volume of the composition that is more acidic than the digested microbe or microbe components can be present at a 1:1, 5:4, 4:3, 3:2, 2:1 ratio to the volume of the digested microbe or microbe components.

In some embodiments of any of the aspects, the composition that is more acidic than the digested microbe or microbe components is present at a concentration of at least 0.5%. As a non-limiting example, the concentration of the composition that is more acidic than the digested microbe or microbe components can be at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, at least 5.0%, at least 6.0%, at least 7.0%, at least 8.0%, at least 9.0%, or at least 10.0%.

In some embodiments of any of the aspects, the composition that is more acidic than the digested microbe or microbe components is selected from the group consisting of trifluoroacetic acid (TFA; CF₃COOH), acetic acid (CH₃COOH), and formic acid (CH₃COOH). As a non-limiting example, the composition that is more acidic than the digested microbe or microbe components can be hydrofluoric acid (HF), phosphoric acid (H₃PO₄), nitrous acid (HNO₂), lactic acid, citric acid, oxalic acid, uric acid, malic acid, or any carboxylic acid (—COOH). As a non-limiting example, the composition that is more acidic than the digested microbe or microbe components can be hydrochloric acid (HCl), nitric acid (HNO₃),—sulfuric acid (H₂SO₄), hydrobromic acid (HBr), hydroiodic acid (HI), perchloric acid (HClO₄), or chloric acid (HClO₃). As a non-limiting example, the composition that is more acidic than the digested microbe or microbe components can be any composition with a pKa below 7.

In some embodiments of any of the aspects, the isolated microbe or microbe components are digested with a protease but not heated and not contacted with a composition that is more acidic than the digested microbe or microbe components. In some embodiments of any of the aspects, the isolated microbe or microbe components are digested with a protease and heated but not contacted with a composition that is more acidic than the digested microbe or microbe components. In some embodiments of any of the aspects, the isolated microbe or microbe components are digested with a protease and contacted with a composition that is more acidic than the digested microbe or microbe components but not heated. In some embodiments of any of the aspects, the isolated microbe or microbe components are digested with a protease, heated, and contacted with a composition that is more acidic than the digested microbe or microbe components. In some embodiments of any of the aspects, the isolated microbe or microbe components are not digested with a protease, not heated, and not contacted with a composition that is more acidic than the digested microbe or microbe components.

In some embodiments of any of the aspects, the sample has not been cultured. In other words, the microbes in the sample have not been allowed to replicate or amplify in a culture medium. Accordingly, in some embodiments of any of the aspects, the methods described herein do not comprise a culturing step, e.g., a step involving culturing and/or maintaining the microbe(s) ex vivo or in vitro. In some embodiments of any of the aspects, the time from the step of collecting the sample to the end of detection takes equal to or less than 90 minutes. As a non-limiting example, the time from the step of collecting the sample to the end of detection takes at most 60 minutes, at most 70 minutes, at most 80 minutes, at most 90 minutes, at most 100 minutes, at most 110 minutes, at most 120 minutes, at most 2.5 hours, at most 3.0 hours, at most 3.5 hours, at most 4.0 hours, at most 4.5 hours, at most 5.0 hours, at most 5.5 hours, at most 6.0 hours, at most 12.0 hours, at most 18 hours, or at most 24 hours.

In some embodiments of any of the aspects, the sample is lysed prior to the step of isolating. The inventors have shown, in some embodiments, that lysing or killing microbes in a sample by mechanical treatment (e.g., beadmilling, sonication, or other functionally equivalent method to disrupt cell wall), and/or chemical treatment (e.g., antibiotics or other antimicrobial agents) can allow detection of encapsulated microbes such as Klebsiella species that would not be otherwise detected. Thus, a simple pre-treatment of a sample to lyse or kill microbes can be performed prior to binding of the PRRs to exposed MAMPs. Therefore, this will not only increase the sensitivity of a PRR-based detection method, but can also surprisingly and significantly increase the spectrum of microbes that can be detected by a PRR-based detection method.

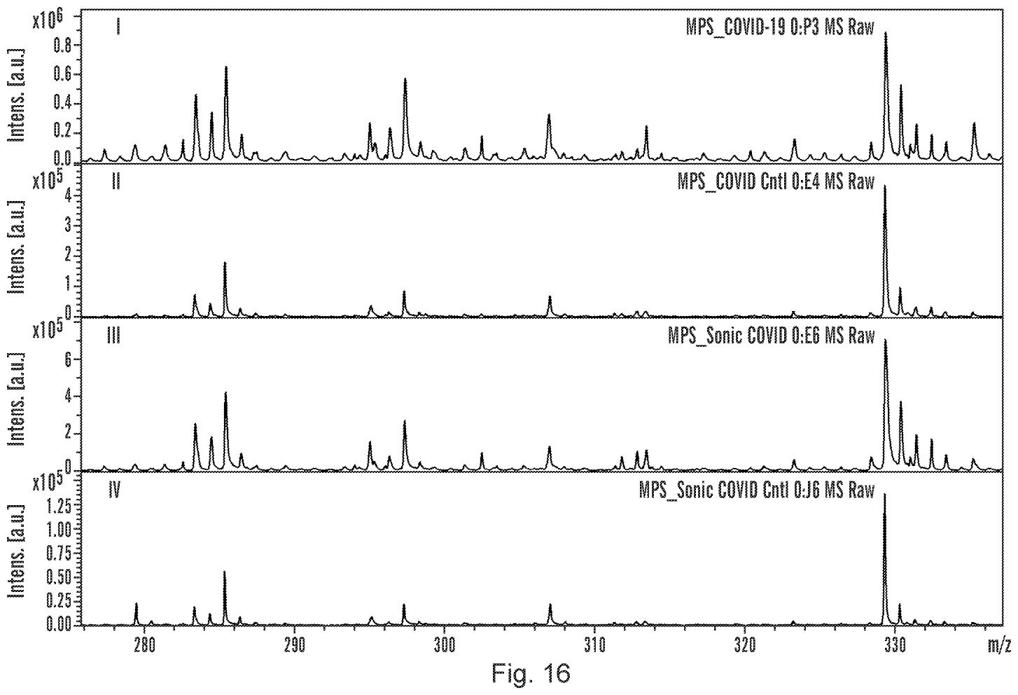

In some embodiments of any of the aspects, MAMPs can be exposed, released or generated from microbes in a sample by various sample pretreatment methods. In some embodiments, the MAMPs can be exposed, released or generated by lysing or killing at least a portion of the microbes in the sample. Without limitations, any means known or available to the practitioner for lysing or killing microbe cells can be used. Exemplary methods for lysing or killing the cells include, but are not limited to, physical, mechanical, chemical, radiation, biological, and the like. Accordingly, pre-treatment for lysing and/or killing the microbe cells can include application of one or more of ultrasound waves, vortexing, sonication, centrifugation, vibration, magnetic field, radiation (e.g., light, UV, Vis, IR, X-ray, and the like), change in temperature, flash-freezing, change in ionic strength, change in pH, incubation with chemicals (e.g. antimicrobial agents), enzymatic degradation, and the like. In some embodiments of any of the aspects, the sample is sonicated prior to the step of isolating (see e.g., FIG. 16). As a non-limiting example, the sample can be sonicated for at least 1 second (s), at least 5 s, at least 10 s, at least 15 s, at least 20 s, at least 25 s, at least 30 s, at least 1 minute (min), at least 2 min, at least 3 min, at least 5 min, at least 6 min, at least 7 min, at least 8 min, at least 9 min, at least 10 min, at least 15 min, at least 20 min, at least 25 min, at least 30, at least 40 min, at least 50 min, or at least 60 min. In some embodiments of any of the aspects, the sample is sonicated at 100% pulse strength, and the pulse strength can be about 20 kHz. In some embodiments of any of the aspects, the pulse strength is at least 1 kHz, at least 5 kHz, at least 10 kHz, at least 15 kHz, at least 20 kHz, at least 25 kHz, at least 30 kHz, at least 35 kHz, at least 40 kHz, at least 45 kHz, or at least 50 kHz). In some embodiments of any of the aspects, the sample is sonicated at about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or about 100% pulse strength.

In some embodiments of any of the aspects, the patient has been treated with antibiotics or an antimicrobial agent. In some embodiments of any of the aspects, the sample contains at least one antibiotic or at least one antimicrobial agent, non-limiting examples of which are described further herein. In some embodiments of any of the aspects, the sample contains at least two antibiotics or at least two antimicrobial agents (see e.g., Example 1).

In some embodiments of any of the aspects, the patient has been treated with antifungals, non-limiting examples of which are described further herein. In some embodiments of any of the aspects, the sample contains antifungals, for example at least 1, at least 2, at least 3, at least 4, or at least 5 antifungals.

In some embodiments of any of the aspects, the patient has been treated with antivirals, non-limiting examples of which are described further herein. In some embodiments of any of the aspects, the sample contains antivirals, for example at least 1, at least 2, at least 3, at least 4, or at least 5 antivirals.

As used herein, the term “antimicrobial agent” (also referred to herein as an antimicrobial, antimicrobial therapeutic, and the like) refers to a molecule or composition which destroys microbes (i.e., bacteria, fungi, viruses, parasites and microbial spores) or prevents or inhibits their development, proliferation and/or pathogenic action. The term “antimicrobial” thus comprises antibacterials, antifungals, and antivirals. Exemplary antimicrobial agents include, but are not limited to, small organic or inorganic molecules; peptides; proteins; peptide analogs and derivatives; peptidomimetics; antibodies (polyclonal or monoclonal); antigen binding fragments of antibodies; nucleic acids; nucleic acid analogs and derivatives; an extract made from biological materials such as bacteria, plants, fungi, or animal cells; animal tissues; naturally occurring or synthetic compositions; and any combinations thereof.

In some embodiments of any of the aspects, the antimicrobial agent can be selected from aminoglycosides, ansamycins, beta-lactams, bis-biguanides, carbacephems, carbapenems, cationic polypeptides, cephalosporins, fluoroquinolones, glycopeptides, iron-sequestering glycoproteins, linosamides, lipopeptides, macrolides, monobactams, nitrofurans, oxazolidinones, penicillins, polypeptides, quaternary ammonium compounds, quinolones, silver compounds, sulfonamides, tetracyclines, and any combinations thereof. In some embodiments of any of the aspects, the antimicrobial agent can comprise an antibiotic.

Some exemplary specific antimicrobial agents include broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefmetazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin W Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

In some embodiments of any of the aspects, the antifungal is selected from the group consisting of: polyene antifungals, Amphotericin B, Candicidin, Filipin, Hamycin, Natamycin, Nystatin, Rimocidin, imidazole antifungals, triazole antifungals, thiazole antifungals, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Triazoles[edit], Albaconazole, Efinaconazole, Epoxiconazole, Fluconazole, Isavuconazole, Itraconazole, Posaconazole, Propiconazole, Ravuconazole, Terconazole, Voriconazole, Abafungin, Allylamines, amorolfin, butenafine, naftifine, terbinafine, Echinocandins, Anidulafungin, Caspofungin, Micafungin, Aurones, Benzoic acid, Ciclopirox, Flucytosine, 5-fluorocytosin, Griseofulvin, Haloprogin, Tolnaftate, Undecylenic acid, Triacetin, Crystal violet, Castellani's paint, Orotomide, Miltefosine, Potassium iodide, Coal tar, Copper(II) sulfate, Selenium disulfide, Sodium thiosulfate, Piroctone olamine, Iodoquinol, clioquinol, Acrisorcin, Zinc pyrithione, and Sulfur. Additional antifungals known in the art can also be used.

In some embodiments of any of the aspects, the antiviral is selected from the group consisting of: Abacavir, Acyclovir, Adefovir, Amantadine, Ampligen, Amprenavir, antiretroviral, Arbidol, Atazanavir, Atripla, Cidofovir, Combivir, Darunavir, Delavirdine, Didanosine, Docosanol, Dolutegravir, Ecoliever, Edoxudine, Efavirenz, Emtricitabine, Enfuvirtide, Entecavir, Famciclovir, Fomivirsen, Fosamprenavir, Foscarnet, Fosfonet, Fusion inhibitor, Ibacitabine, Idoxuridine, Imiquimod, Imunovir, Indinavir, Inosine, Integrase inhibitor, Interferon, Interferon type I, Interferon type II, Interferon type III, Lamivudine, Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Penciclovir, Peramivir, Pleconaril, Podophyllotoxin, Protease inhibitor, Pyramidine, Raltegravir, Reverse transcriptase inhibitor, Ribavirin, Rimantadine, Ritonavir, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine. Additional antivirals known in the art can also be used.

Without limitations, incubation of microbes present in the sample with one or more antimicrobial agents can be at any desired temperature and for any desired duration. In some embodiments of any of the aspects, the incubation can be performed at room temperature or at an elevated temperature. In some embodiments of any of the aspects, incubation can be performed at a temperature of about 30° C. to about 45° C. In one embodiment, incubation can be performed at a temperature of about 37° C.

As indicated above, incubation of microbes present in a sample can be performed for any desired time period, which can vary with a number of factors, including but not limited to, temperature of incubation, concentration of microbes in the sample, and/or potency and/or concentrations of antimicrobial agents used. In some embodiments of any of the aspects, incubation can be for about at least one minute (e.g. one, five, ten, fifteen, twenty, twenty-five, thirty, thirty-five, forty, forty-five, fifty-five, sixty, ninety minutes or more). In some embodiments of any of the aspects, incubation can be for at least about one hour, at least about two hours, at least about three hours, at least about four hours, at least about five hours, at least about six hours, at least about seven hours, at least about eight hours, at least about nine hours, at least about ten hours or more. In some embodiments of any of the aspects, incubation can be for a period of about fifteen minutes to about ninety minutes. In one embodiment, incubation can be for a period of about thirty minutes to about sixty minutes. In another embodiment, incubation can be for a period of about thirty minutes to about twenty-four hours. In one embodiment, incubation can be for a period of at least about four hours.

Amount of one or more antimicrobial agent added to a sample can be any desired amount and vary with a number of factors, including but not limited to, types of microbes in the sample, and/or potency of antimicrobial agents used. For example, one or more antimicrobial agents added to sample can have a concentration ranging from nanomolars to millimolars. In some embodiments of any of the aspects, one or more antimicrobial agents added to a sample can have a concentration ranging from 0.01 nM to about 100 mM, from about 0.01 nM to about 10 mM, or from about 0.1 nM to about 1 mM.

In some embodiments of any of the aspects, one or more antimicrobial agents added to a sample can have a concentration ranging from nanograms per milliliters to micrograms per milliliters. In some embodiments of any of the aspects, one or more antimicrobial agents added to a sample can have a concentration ranging from about 1 ng/mL to about 1000 μg/mL, from about 10 ng/mL to about 750 μg/mL, or from about 100 ng/mL to about 500 μg/mL. In some embodiments of any of the aspects, one or more antimicrobial agents added to a sample can have a concentration ranging from about 10 μg/mL to about 500 μg/mL or from about 100 μg/mL to about 500 μg/mL.

In some embodiments of any of the aspects, the pre-treatment can comprise incubating the sample with at least one or more degradative enzymes. For example, in some embodiments of any of the aspects, a degradative enzyme can be selected to cleave at least some of the cell wall carbohydrates, thus restoring detection of carbohydrates that are otherwise not recognized by PRRs. In some embodiments of any of the aspects, a degradative enzyme can be selected to cause call wall degradation and thus release or expose MAMPs that are otherwise unable bind to the PRRs. Other examples of degradative enzymes include, but are not limited to, proteases, lipases such as phospholipases, neuraminidase, and/or sialidase, or any other enzyme modifying the presentation of any MAMP to any PRR leveraged for detection of the MAMP. For instance, an exemplary PRR can comprise MBL or recombinant human MBL or engineered FcMBL, which binds mannose containing carbohydrates such as the core of LPS, the Wall Teichoic Acid from Staphylococcus aureus, PIM6 or Mannose-capped LipoArabinoMannan from M. tuberculosis whereas CRP binds phosphocholine found in Streptococcus pneumonia (Brundish and Baddiley, 1968), Haemophilus influenzae (Weiser et al., 1997), Pseudomonas aeruginosa, Neisseria meningitides, Neisseria gonorrhoeae (Serino and Virji, 2000), Morganella morganii (Potter, 1971), and Aspergillus fumigatus (Volanakis, “Human C-reactive protein: expression, structure, and function, “Molecular Immunology,” 2001, 38(2-3): 189-197). Other PRR can be equally leveraged to recognize MAMPs such as NODs or PGRP.

Described herein are methods of detecting at least one microbe and/or at least microbe component in a sample. In some embodiments of any of the aspects, the sample comprises at least 1, at least 2, at least 3, at least 4, or at least 5 types of microbes, as described further herein (e.g., a coinfection). In some embodiments of any of the aspects, the sample comprises at least 1, at least 2, at least 3, at least 4, or at least 5 types of microbe components, as described further herein (e.g., multiple components from at least 1 microbe). In some embodiments of any of the aspects, the sample comprises blood, serum, plasma, sputum, urine, joint fluid, or any other tissue or biological sample.

In some embodiments of any of the aspects, such methods can be applied to detect a microbial infection in a sample, including not limited to a patient sample, an animal model sample, an environmental sample, or a non-biological sample.

The term “sample” or “test sample” as used herein can denote a sample taken or isolated from a biological organism, e.g., a blood or plasma sample from a subject. In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

The test sample can be obtained by removing a sample from a subject, but can also be accomplished by using a previously isolated sample (e.g. isolated at a prior time point and isolated by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods, assays, and systems described herein can further comprise a step of obtaining or having obtained a test sample from a subject. In some embodiments of any of the aspects, the subject can be a human subject. In some embodiments of any of the aspects, the subject can be a subject in need of treatment for (e.g. having or diagnosed as having) a microbial infection or a subject at risk of or at increased risk of developing a microbial infection as described elsewhere herein.

In some embodiments of any of the aspects, the sample obtained from a subject can be a biopsy sample. In some embodiments of any of the aspects, the sample obtained from a subject can be a blood or serum sample. In some embodiments of any of the aspects, the sample obtained from a subject can be a sample of tissue, material, or fluid which exhibits signs of microbial infection, e.g., inflammation, swelling, pus, necrosis, etc.

Without limitations a sample for use in the various aspects disclosed herein can be a liquid, supercritical fluid, solutions, suspensions, gases, gels, slurries, and combinations thereof. The test sample or fluid can be aqueous or non-aqueous. In some embodiments of any of the aspects, the sample can be an aqueous fluid. As used herein, the term “aqueous fluid” refers to any flowable water-containing material that is suspected of comprising a pathogen.

The sample can be collected from any source, including, e.g., human, animal, plants, environment, or organic or inorganic materials, suspected of being infected or contaminated by microbe(s).

In some embodiments of any of the aspects, the sample can be a biological sample. As used herein, the term “biological sample” denotes all materials that are produced by biological organisms or can be isolated or obtained from them. The term “biological sample” includes untreated or pretreated samples. Pretreated biological samples can be, for example, heat treated (frozen, dried, etc.) or chemically treated (e.g., fixed in suitable chemicals such as formalin, alcohol, etc.)

In some embodiments of any of the aspects, the biological sample can be a biological fluid. Exemplary biological fluids can include, but are not limited to, blood (including whole blood, plasma, cord blood and serum), lactation products (e.g., milk), amniotic fluids, sputum, saliva, urine, semen, joint fluid, cerebrospinal fluid, bronchial aspirate, perspiration, mucus, liquefied feces, synovial fluid, lymphatic fluid, tears, tracheal aspirate, and fractions thereof. In some embodiments of any of the aspects, a biological fluid can include a homogenate of a tissue specimen (e.g., biopsy) from a subject. In some embodiments of any of the aspects, a sample can comprise a suspension obtained from homogenization of a solid sample obtained from a solid organ or a fragment thereof.

In some embodiments of the methods disclosed herein, the method comprises obtaining a sample from the subject. Methods of obtaining a sample from a subject are well known in the art and easily available to one of skill in the art.

In some embodiments of any of the aspects, the sample can include a fluid or specimen obtained from an environmental source, e.g., but not limited to, food products or industrial food products, food produce, poultry, meat, fish, beverages, dairy products, water supplies (including wastewater), surfaces, ponds, rivers, reservoirs, swimming pools, soils, food processing and/or packaging plants, agricultural places, hydrocultures (including hydroponic food farms), pharmaceutical manufacturing plants, animal colony facilities, and any combinations thereof.

In some embodiments of any of the aspects, the sample can include a fluid (e.g., culture medium) from a biological culture. Examples of a fluid (e.g., culture medium) obtained from a biological culture includes the one obtained from culturing or fermentation, for example, of single- or multi-cell organisms, including prokaryotes (e.g., bacteria) and eukaryotes (e.g., animal cells, plant cells, yeasts, fungi), and including fractions thereof. In some embodiments of any of the aspects, the test sample can include a fluid from a blood culture. In some embodiments of any of the aspects, the culture medium can be obtained from any source, e.g., without limitations, research laboratories, pharmaceutical manufacturing plants, hydrocultures (e.g., hydroponic food farms), diagnostic testing facilities, clinical settings, and any combinations thereof.

In some embodiments of any of the aspects, the test sample can include a media or reagent solution used in a laboratory or clinical setting, such as for biomedical and molecular biology applications. As used herein, the term “media” refers to a medium for maintaining a tissue, an organism, or a cell population, or refers to a medium for culturing a tissue, an organism, or a cell population, which contains nutrients that maintain viability of the tissue, organism, or cell population, and support proliferation and growth. As used herein, the term “reagent” refers to any solution used in a laboratory or clinical setting for biomedical and molecular biology applications. Reagents include, but are not limited to, saline solutions; PBS solutions; buffered solutions, such as phosphate buffers, EDTA, Tris solutions; and any combinations thereof.

In some embodiments of any of the aspects, the sample can a non-biological sample. Non-limiting examples of non-biological samples include a fomite (i.e., an object or material which is likely to carry infection), clothing, housing materials, food utensils, or any non-biological material that is suspected to have come into contact with a microbe or a microbe component. In some embodiments of any of the aspects, the sample can be a non-biological fluid. As used herein, the term “non-biological fluid” refers to any fluid that is not a biological fluid as the term is defined herein. Non-limiting examples of a non-biological fluid include water (e.g., in the environment or in a plumbing device), food liquids, or any non-biological fluid that is suspected to have come into contact with a microbe or a microbe component.

Described herein are methods of detecting at least one microbe or at least one microbe component. As used herein, the term “microbes” or “microbe” generally refers to microorganism(s), including bacteria, virus, fungi, parasites, protozoan, archaea, protists, e.g., algae, and a combination thereof. The term “microbes” encompasses both live and dead microbes. The term “microbes” also includes pathogenic microbes or pathogens, e.g., bacteria causing diseases such as plague, tuberculosis and anthrax; protozoa causing diseases such as malaria, sleeping sickness and toxoplasmosis; fungi causing diseases such as ringworm, candidiasis or histoplasmosis; and bacteria causing diseases such as sepsis.

In some embodiments of any of the aspects, the microbe is a human pathogen, in other words a microbe that causes at least one disease in a human. In some embodiments of any of the aspects, the sample contains at least one pathogen. In some embodiments of any of the aspects, the sample contains more than one pathogen. As a non-limiting example, the sample can comprise at least 1, at least 2, at least 3, at least 4, or at least 5 pathogens (e.g., a co-infection).

In some embodiments of any of the aspects, the at least one microbe comprises a Gram-positive bacterial species, a Gram-negative bacterial species, a mycobacterium, a fungus, a parasite, and/or a virus. In some embodiments of any of the aspects, the Gram-positive bacterial species comprises bacteria from the class Bacilli. In some embodiments of any of the aspects, the Gram-negative bacterial species comprises bacteria from the class Gammaproteobacteria. In some embodiments of any of the aspects, the mycobacterium comprises bacteria from the class Actinobacteria. In some embodiments of any of the aspects, the fungus comprises fungus from the class Saccharomycetes.

In some embodiments of any of the aspects, the at least one microbe is selected from the group consisting of Staphylococcus aureus, Streptococcus pyogenes, Klebsiella pneumoniae, Pseudomonas aeruginosa, Mycobacterium tuberculosis, Candida albicans, or Escherichia coli. In some embodiments of any of the aspects, the at least one microbe is selected from the group consisting of S. aureus strain 3518, S. pyogenes strain 011014, K. pneumoniae strain 631, E. coli strain 41949, P. aeruginosa strain 41504, C. albicans strain 1311, M. tuberculosis strain H37Rv.

In some embodiments of any of the aspects, the following microbes that causes diseases and/or associated microbial matter can be amendable to the methods of various aspects described herein: Bartonella henselae, Borrelia burgdorferi, Campylobacter jejuni, Campylobacter fetus, Chlamydia trachomatis, Chlamydia pneumoniae, Chylamydia psittaci, Simkania negevensis, Escherichia coli (e.g., 0157:H7 and K88), Ehrlichia chafeensis, Clostridium botulinum, Clostridium perfringens, Clostridium tetani, Enterococcus faecalis, Haemophilius influenzae, Haemophilius ducreyi, Coccidioides immitis, Bordetella pertussis, Coxiella burnetii, Ureaplasma urealyticum, Mycoplasma genitalium, Trichomatis vaginalis, Helicobacter pylori, Helicobacter hepaticus, Legionella pneumophila, Mycobacterium tuberculosis, Mycobacterium bovis, Mycobacterium africanum, Mycobacterium leprae, Mycobacterium asiaticum, Mycobacterium avium, Mycobacterium celatum, Mycobacterium celonae, Mycobacterium fortuitum, Mycobacterium genavense, Mycobacterium haemophilum, Mycobacterium intracellulare, Mycobacterium kansasii, Mycobacterium malmoense, Mycobacterium marinum, Mycobacterium scrofulaceum, Mycobacterium simiae, Mycobacterium szulgai, Mycobacterium ulcerans, Mycobacterium xenopi, Corynebacterium diptheriae, Rhodococcus equi, Rickettsia aeschlimannii, Rickettsia africae, Rickettsia conorii, Arcanobacterium haemolyticum, Bacillus anthracia, Bacillus cereus, Lysteria monocytogenes, Yersinia pestis, Yersinia enterocolitica, Shigella dysenteriae, Neisseria meningitides, Neisseria gonorrhoeae, Streptococcus bovis, Streptococcus hemolyticus, Streptococcus mutans, Streptococcus pyogenes, Streptococcus pneumoniae, Staphylococcus aureus, Staphylococcus epidermidis, Staphylococcus pneumoniae, Staphylococcus saprophyticus, Vibrio cholerae, Vibrio parahaemolyticus, Salmonella typhi, Salmonella paratyphi, Salmonella enteritidis, Treponema pallidum, Human rhinovirus, Human coronavirus (e.g., SARS-CoV, SARS-CoV-2), Dengue virus, Filoviruses (e.g., Marburg and Ebola viruses), Hantavirus, Rift Valley virus, Hepatitis B, C, and E, Human Immunodeficiency Virus (e.g., HIV-1, HIV-2), HHV-8, Human papillomavirus, Herpes virus (e.g., HV-I and HV-II), Human T-cell lymphotrophic viruses (e.g., HTLV-I and HTLV-II), Bovine leukemia virus, Influenza virus, Guanarito virus, Lassa virus, Measles virus, Rubella virus, Mumps virus, Chickenpox (Varicella virus), Monkey pox, Epstein Bahr virus, Norwalk (and Norwalk-like) viruses, Rotavirus, Parvovirus B19, Hantaan virus, Sin Nombre virus, Venezuelan equine encephalitis, Sabia virus, West Nile virus, Yellow Fever virus, causative agents of transmissible spongiform encephalopathies, Creutzfeldt-Jakob disease agent, variant Creutzfeldt-Jakob disease agent, Candida, Cryptcooccus, Cryptosporidium, Giardia lamblia, Microsporidia, Plasmodium vivax, Pneumocystis carinii, Toxoplasma gondii, Trichophyton mentagrophytes, Enterocytozoon bieneusi, Cyclospora cayetanensis, Encephalitozoon hellem, Encephalitozoon cuniculi, among other viruses, bacteria, archaea, protozoa, and fungi. Microbes disclosed in the Examples are also amenable to the methods of various aspects described herein. In yet other embodiments, bioterror agents (e.g., B. anthracis, and smallpox) can be amendable to the methods of various aspects described herein.

In some embodiments of any of the aspects, the microbe is a coronavirus. The scientific name for coronavirus is Orthocoronavirinae or Coronavirinae. Coronaviruses belong to the family of Coronaviridae, order Nidovirales, and realm Riboviria. They are divided into alphacoronaviruses and betacoronaviruses which infect mammals—and gammacoronaviruses and deltacoronaviruses which primarily infect birds. Non limiting examples of alphacoronaviruses include: Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, and Feline Infectious Peritonitis Virus (FIPV, also referred to as Feline Infectious Hepatitis Virus). Non limiting examples of betacoronaviruses include: Betacoronavirus 1 (e.g., Bovine Coronavirus, Human coronavirus 0C43), Human coronavirus HKU1, Murine coronavirus (also known as Mouse hepatitis virus (MHV)), Pipistrellus bat coronavirus HKU5, Rousettus bat coronavirus HKU9, Severe acute respiratory syndrome-related coronavirus (e.g., SARS-CoV, SARS-CoV-2), Tylonycteris bat coronavirus HKU4, Middle East respiratory syndrome (MERS)-related coronavirus, and Hedgehog coronavirus 1 (EriCoV). Non limiting examples of gammacoronaviruses include: Beluga whale coronavirus SW1, and Infectious bronchitis virus. Non limiting examples of deltacoronaviruses include: Bulbul coronavirus HKU11, and Porcine coronavirus HKU15. In some embodiments of any of the aspects, the microbe is severe acute respiratory syndrome coronavirus 2 (SARS-CoV-2), which causes coronavirus disease of 2019 (COVID19 or simply COVID); see e.g., FIG. 16.

In some embodiments of any of the aspects, microbe components can be detected with a method as described herein. As used herein, “microbe component” refers to any part of a microbe such as cell wall components, cell membrane components, cytosolic components, intracellular components, nucleic acid, or organelles in the case of eukaryotic microbes. In some embodiments of any of the aspects, the microbial component comprises a component from a Gram-positive bacterial species, a Gram-negative bacterial species, a mycobacterium, a fungus, a parasite, a virus, or any microbe described herein or known in the art.

In some embodiments of any of the aspects, the microbe components comprise microbe-associated molecular patterns (MAMPs). As used herein and throughout the specification, the term “microbe associated molecular patterns” or “MAMPs” refers to molecules, components or motifs associated with or secreted or released by microbes or groups of microbes (whole and/or lysed and/or disrupted) that are generally recognized by corresponding pattern recognition receptors (PRRs). In some embodiments of any of the aspects, the MAMPs can also encompass molecules associated with cell components released during cell damage or lysis. Examples of MAMPs include, but are not limited to, microbial carbohydrates (e.g., lipopolysaccharide or LPS, mannose), endotoxins, microbial nucleic acids (e.g., bacterial or viral DNA or RNA; e.g., nucleic acid comprising a CpG site), microbial peptides (e.g., flagellin), peptidoglycans, lipoteichoic acids, N-formylmethionine, lipoproteins, lipids, phospholipids or their precursors (e.g., phosphochloline), and fungal glucans. In some embodiments of any of the aspects, microbe components comprise cell wall and membrane components known as pathogen-associated molecular patterns (PAMPs) including lipopolysaccharide (LPS) endotoxin, lipoteichoic acid, and attached or released outer membrane vesicles.

In some embodiments of any of the aspects, MAMPs include carbohydrate recognition domain (CRD)-binding motifs. As used herein, the term “carbohydrate recognition domain (CRD)-binding motifs” refers to molecules or motifs that bind to a molecule or composition comprising a CRD. As used herein, the term “carbohydrate recognition domain” or “CRD” refers to one or more regions, at least a portion of which, can bind to carbohydrates on a surface of microbes or pathogens. In some embodiments of any of the aspects, the CRD can be derived from a lectin described herein. In some embodiments of any of the aspects, the CRD can be derived from a mannan-binding lectin (MBL). Accordingly, in some embodiments of any of the aspects, MAMPs are molecules, components or motifs associated with microbes or groups of microbes that are recognized by lectin-based PRRs described herein. In one embodiment, MAMPs are molecules, components, or motifs associated with microbes or groups of microbes that are recognized by mannan-binding lectin (MBL).

In some embodiments of any of the aspects, MAMPs are molecules, components or motifs associated with microbes or groups of microbes that are recognized by a C-reactive protein (CRP)-based PRR.

As used herein and throughout the specification, the term “pattern recognition receptors” or “PRRs” refer to microbe-binding domains, molecules, proteins or peptides that bind to at least one or more (including, at least two, at least three, at least four, at least five, or more) MAMPs described herein. In some embodiments of any of the aspects, a PRR can be a naturally occurring or synthetic molecule. In some embodiments of any of the aspects, a PRR can be a recombinant molecule. In some embodiments of any of the aspects, a PRR can be a fusion protein. For example, PRR can also be fusion protein comprising at least a part of a lectin and at least a part of a second protein or peptide, e.g., but not limited to an Fc portion of an immunoglobulin or another microbe-binding molecule. An exemplary lectin can be mannan binding lectin (MBL) or other mannan binding molecules. Non-limiting examples of PRR include at least a microbe-binding domain selected from lectins (e.g., C-type lectins such as mannan binding lectin (MBL)), toll-like receptors, NODs, complement receptors, collectins, ficolins, pentraxins such as serum amyloid and C-reactive protein, lipid transferases, peptidoglycan recognition proteins (PGRs), and any combinations thereof. In some embodiments of any of the aspects, PRRs can be microbe-binding molecules described in the International Patent Application No. WO 2013/012924, the content of which is incorporated by reference in its entirety.

The MAMPs can be exposed, released or generated from microbes in a sample by various sample pretreatment methods. In some embodiments of any of the aspects, the MAMPs can be exposed, released or generated by lysing or killing at least a portion of the microbes in the sample. Without limitations, any means known or available to the practitioner for lysing or killing microbe cells can be used. Exemplary methods for lysing or killing the cells include, but are not limited to, physical, mechanical, chemical, radiation, biological, and the like. Accordingly, pre-treatment for lysing and/or killing the microbe cells can include application of one or more of ultrasound waves, vortexing, centrifugation, vibration, magnetic field, radiation (e.g., light, UV, Vis, IR, X-ray, and the like), change in temperature, flash-freezing, change in ionic strength, change in pH, incubation with chemicals (e.g. antimicrobial agents), enzymatic degradation, and the like.

In some embodiments of any of the aspects, the drug sensitivity of the pathogen is identified. Accordingly, in some embodiments of any of the aspects, the detection method further comprises determining whether at least one of the microbes detected in the sample is sensitive or resistant to an antimicrobial therapeutic. As a non-limiting example, the detection methods described herein can be used to determine the presence or absence of an antimicrobial resistance marker or an antimicrobial susceptibility marker. In some embodiments of any of the aspects, the presence of an antimicrobial resistance marker and/or the absence of an antimicrobial susceptibility marker can indicate that the at least one microbe in a sample is resistant to that specific antimicrobial. In some embodiments of any of the aspects, the absence of an antimicrobial resistance marker and/or the presence of an antimicrobial susceptibility marker can indicate that the at least one microbe in a sample is susceptible to that specific antimicrobial.

As used herein “antibiotic resistance marker” refers to a gene product, mRNA, polypeptide, polypeptide variant, or other macromolecule that confers resistance to a specific antimicrobial, such as by enzymatically cleaving the antimicrobial or specifically effluxing the antimicrobial. In some embodiments of any of the aspects, non-limiting examples of antimicrobial resistance markers include Aminocoumarin-resistant DNA topoisomerases (e.g., Aminocoumarin-resistant GyrB, ParE, ParY); Aminoglycoside acetyltransferases (e.g., AAC(1), AAC(2′), AAC(3), AAC(6′)); Aminoglycoside nucleotidyltransferases (e.g., ANT(2″), ANT(3″), ANT(4′), ANT(6), ANT(9)); Aminoglycoside phosphotransferases (e.g., APH(2″), APH(3″), APH(3′), APH(4), APH(6), APH(7″), APH(9)); 16S rRNA methyltransferases (e.g., ArmA, RmtA, RmtB, RmtC, Sgm); Class A β-lactamases (e.g., AER, BLA1, CTX-M, KPC, SHV, TEM, etc.); Class B (metallo-)β-lactamases (e.g., BlaB, CcrA, IMP, NDM, VIM, etc.); Class C β-lactamases (e.g., ACT, AmpC, CMY, LAT, PDC, etc.); Class D β-lactamases (e.g., OXA β-lactamase); mecA (methicillin-resistant PBP2); mutant porin proteins conferring antibiotic resistance; antibiotic-resistant Omp36, antibiotic-resistant OmpF, antibiotic-resistant PIB (por); genes modulating β-lactam resistance (e.g., bla (blaI, blaR1) and mec (mecI, mecR1) operons); Chloramphenicol acetyltransferase (CAT); Chloramphenicol phosphotransferase; Ethambutol-resistant arabinosyltransferase (EmbB); Mupirocin-resistant isoleucyl-tRNA synthetases (e.g., MupA, MupB); resistance markers for peptide antibiotics, including but not limited to integral membrane protein MprF; resistnace markers for phenicol, including but not limited to Cfr 23S rRNA methyltransferase; Rifampin ADP-ribosyltransferase (Arr); Rifampin glycosyltransferase; Rifampin monooxygenase; Rifampin phosphotransferase; Rifampin resistance RNA polymerase-binding proteins (e.g., DnaA, RbpA); Rifampin-resistant beta-subunit of RNA polymerase (RpoB); resistance markers against Streptogramins; Cfr 23S rRNA methyltransferase; Erm 23S rRNA methyltransferases (e.g., ErmA, ErmB, Erm(31), etc.); Streptogramin resistance ATP-binding cassette (ABC) efflux pumps (e.g., Lsa, MsrA, Vga, VgaB); Streptogramin Vgb lyase; Vat acetyltransferase; Fluoroquinolone acetyltransferase; Fluoroquinolone-resistant DNA topoisomerases; Fluoroquinolone-resistant GyrA, Fluoroquinolone-resistant GyrB, Fluoroquinolone-resistant ParC; Quinolone resistance protein (Qnr); Fosfomycin phosphotransferases (e.g., FomA, FomB, FosC); Fosfomycin thiol transferases (e.g., FosA, FosB, FosX); resistance markers against Glycopeptides, including not limited to VanA, VanB, VanD, VanR, VanS, etc.; resistance markers against Lincosamides; Cfr 23S rRNA methyltransferase; Erm 23S rRNA methyltransferases (e.g., ErmA, ErmB, Erm(31), etc.); Lincosamide nucleotidyltransferase (Lin); resistance markers against Linezolid; Cfr 23S rRNA methyltransferase; resistance markers against Macrolides, such as Cfr 23S rRNA methyltransferase, Erm 23S rRNA methyltransferases (e.g., ErmA, ErmB, Erm(31), etc.); Macrolide esterases (e.g., EreA, EreB); Macrolide glycosyltransferases (e.g., GimA, Mgt, Ole); Macrolide phosphotransferases (MPH) (e.g., MPH(2′)-I, MPH(2′)-II); Macrolide resistance efflux pumps (e.g., MefA, MefE, Mel); Streptothricin acetyltransferase (sat); Sulfonamide-resistant dihydropteroate synthases (e.g., Sul1, Sul2, Sul3, sulfonamide-resistant FolP); resistance markers against Tetracyclines; mutant porin PIB (por) with reduced permeability; Tetracycline inactivation enzyme TetX; Tetracycline resistance major facilitator superfamily (MFS) efflux pumps (e.g., TetA, TetB, TetC, Tet30, Tet31, etc.); Tetracycline resistance ribosomal protection proteins (e.g., TetM, TetO, TetQ, Tet32, Tet36, etc.); efflux pumps conferring antibiotic resistance: ABC antibiotic efflux pump (e.g., MacAB-TolC, MsbA, MsrA, VgaB, etc.); MFS antibiotic efflux pump (e.g., EmrD, EmrAB-TolC, NorB, GepA, etc.); multidrug and toxic compound extrusion (MATE) transporter (e.g., MepA); resistance-nodulation-cell division (RND) efflux pump (e.g., AdeABC, AcrD, MexAB-OprM, mtrCDE, etc.); small multidrug resistance (SMR) antibiotic efflux pump (e.g., EmrE); genes modulating antibiotic efflux (e.g., adeR, acrR, baeSR, mexR, phoPQ, mtrR, etc.). See e.g., MacAuthur et al., Antimicrob Agents Chemother. 2013 July; 57(7):3348-57, which is incorporated herein by reference. In some embodiments of any of the aspects, an antimicrobial resistance marker can include any protein, polypeptide, polypeptide variant, or other macromolecule known in the art to confer resistance to a specific antimicrobial or family of antimicrobials.

As used herein “antibiotic susceptibility marker” refers to a gene product, mRNA, polypeptide, polypeptide variant, or other macromolecule that confers susceptibility to a specific antimicrobial, especially in a domain at fashion. In some embodiments of any of the aspects, an antibiotic susceptibility marker can include any mutant or variant of one of the aforementioned antibiotic resistance markers comprising a mutation that reduces or eliminates the antibiotic resistance. In some embodiments of any of the aspects, non-limiting examples of antimicrobial susceptibility markers include RpsL and GyrA conferring sensitivity in a dominant fashion to two antibiotics, streptomycin and nalidixic acid, respectively (see e.g., Edgar et al., Appl Environ Microbiol. 2012 February; 78(3): 744-751). In some embodiments of any of the aspects, an antimicrobial susceptibility marker can include any protein, polypeptide, polypeptide variant, or other macromolecule known in the art to confer susceptibly to a specific antimicrobial or family of antimicrobials.

In some embodiments of any of the aspects, a therapy model can be provided to the patient and/or physician based on the infection category assigned to the patient. In some embodiments of any of the aspects, a therapy model can be provided to the patient and/or physician based on at least one identified microbe or at least one identified pathogen assigned to the patient. In some embodiments of any of the aspects, the therapy model can comprise treatment with a therapeutic agent specific to the identified microbe or identified pathogen. Any therapeutic agent known in the art to be specific to the identified microbe or identified pathogen can be provided. As used herein “specific to the identified microbe or identified pathogen” refers to an agent that exhibits a killing, growth-halting, or otherwise disabling effect on a specific microbe or group of microbes with no or minimal effect on the patient. Examples of a therapeutic agent specific to the microbe or pathogen include an antibacterial for an identified bacterial species or strain, an antifungal for an identified fungal species or strain, an antiviral for an identified viral species or strain, and/or an anti-parasitic for an identified parasite species or strains. Specific examples of antibiotics, antimicrobials, antibacterials, antivirals, anti-parasitics are well known in the art and are described further herein.

In some embodiments of any of the aspects, the therapy model can comprise at least one therapeutic agent specific to the identified microbe or identified pathogen. In some embodiments of any of the aspects, multiple therapeutic agents can be effective or specific against a microbe. In some embodiments of any of the aspects, the therapy model can comprise 1 therapeutic agent, 2 therapeutic agents, 3 therapeutic agents, 4 therapeutic agents, 5 therapeutic agents, 6 therapeutic agents, 7 therapeutic agents, 8 therapeutic agents, 9 therapeutic agents, or at least 10 therapeutic agents specific to the identified microbe or identified pathogen. In some embodiments of any of the aspects, the therapy model can comprise cycling multiple therapeutic agents.

In some embodiments of any of the aspects, the therapeutic agent specific to the identified microbe or identified pathogen provided to the patient can be the same therapeutic agent administered to the patent prior to the isolation, detection, and/or identification of the microbe by the methods as described herein. In such cases, the dosage of the therapeutic agent can be decreased or increased, and/or the frequency of administration of the therapeutic agent can be decreased or increased. In some embodiments of any of the aspects, the therapeutic agent specific to the identified microbe or identified pathogen provided to the patient can be different than the therapeutic agent(s) administered to the patent prior to the isolation, detection, and/or identification of the microbe by the methods as described herein. As such, the therapy model can comprise adding, removing, or substituting therapeutic agents that were previously administered to the patient.

In some embodiments of any of the aspects, at least two microbes or at least two pathogens can be identified in a patient sample (e.g., a coinfection). A therapy model can comprise administering at least one therapeutic agent for each identified microbe or for each identified pathogen. In some embodiments of any of the aspects, one therapeutic agent can be effective or specific against multiple microbes and can be administered to the patient.

In some embodiments of any of the aspects, the antimicrobial sensitivity of a specific microbe can be determined by methods as described herein, and the therapy model can be adjusted accordingly. As a non-limiting example, if a microbe is determined to be sensitive to a specific therapeutic agent, then the patient can be administered the specific therapeutic agent and/or the level or administration frequency of the specific therapeutic can be decreased. As a non-limiting example, if a microbe is determined to be resistant to a specific therapeutic agent, then the patient can be administered a different therapeutic agent and/or the level or administration frequency of the specific therapeutic can be increased.

As described herein, levels of at least one microbe or microbe component can be increased in a microbial infection (e.g., sepsis) and/or in subjects with a microbial infection. Accordingly, in one aspect of any of the embodiments, described herein is a method of treating a microbial infection in a subject in need thereof, the method comprising administering at least one therapeutic agent specific to the microbe to a subject determined to have a level of at least one microbe or microbe component that is increased relative to a reference. In one aspect of any of the embodiments, described herein is a method of treating a microbial infection in a subject in need thereof, the method comprising: a) determining the level of at least one microbe or microbe component in a sample obtained from a subject; and b) administering at least one therapeutic agent specific to the microbe to the subject if the level of at least one microbe or microbe component is increased relative to a reference.

In some embodiments of any of the aspects, the method comprises administering at least one therapeutic agent specific to the microbe to a subject previously determined to have a level of at least one microbe or microbe component that is increased relative to a reference. In some embodiments of any of the aspects, described herein is a method of treating a microbial infection in a subject in need thereof, the method comprising: a) first determining the level of at least one microbe or microbe component in a sample obtained from a subject; and b) then administering at least one therapeutic agent specific to the microbe to the subject if the level of at least one microbe or microbe component is increased relative to a reference.

In one aspect of any of the embodiments, described herein is a method of treating a microbial infection in a subject in need thereof, the method comprising: a) determining if the subject has an increased level of at least one microbe or microbe component; and b) administering at least one therapeutic agent specific to the microbe to the subject if the level of at least one microbe or microbe component is increased relative to a reference. In some embodiments of any of the aspects, the step of determining if the subject has an increased level of at least one microbe or microbe component can comprise i) obtaining or having obtained a sample from the subject and ii) performing or having performed an assay on the sample obtained from the subject to determine/measure the level of at least one microbe or microbe component in the subject. In some embodiments of any of the aspects, the step of determining if the subject has an increased level of at least one microbe or microbe component can comprise performing or having performed an assay on a sample obtained from the subject to determine/measure the level of at least one microbe or microbe component in the subject. In some embodiments of any of the aspects, the step of determining if the subject has an increased level of at least one microbe or microbe component can comprise ordering or requesting an assay on a sample obtained from the subject to determine/measure the level of at least one microbe or microbe component in the subject. In some embodiments of any of the aspects, the step of determining if the subject has an increased level of at least one microbe or microbe component can comprise receiving the results of an assay on a sample obtained from the subject to determine/measure the level of at least one microbe or microbe component in the subject. In some embodiments of any of the aspects, the step of determining if the subject has an increased level of at least one microbe or microbe component can comprise receiving a report, results, or other means of identifying the subject as a subject with an increased level of at least one microbe or microbe component.

In one aspect of any of the embodiments, described herein is a method of treating a microbial infection in a subject in need thereof, the method comprising: a) determining if the subject has an increased level of at least one microbe or microbe component; and b) instructing or directing that the subject be administered at least one therapeutic agent specific to the microbe if the level of at least one microbe or microbe component is increased relative to a reference. In some embodiments of any of the aspects, the step of determining if the subject has an increased level of at least one microbe or microbe component can comprise i) obtaining or having obtained a sample from the subject and ii) performing or having performed an assay on the sample obtained from the subject to determine/measure the level of at least one microbe or microbe component in the subject. In some embodiments of any of the aspects, the step of determining if the subject has an increased level of at least one microbe or microbe component can comprise performing or having performed an assay on a sample obtained from the subject to determine/measure the level of at least one microbe or microbe component in the subject. In some embodiments of any of the aspects, the step of determining if the subject has an increased level of at least one microbe or microbe component can comprise ordering or requesting an assay on a sample obtained from the subject to determine/measure the level of at least one microbe or microbe component in the subject. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

Another aspect of the technology described herein relates to kits for detecting at least one microbe or at least one microbe component, among others. Described herein are kit components that can be included in one or more of the kits described herein.

In some embodiments of any of the aspects, the kit comprises an effective amount of an engineered microbe targeting molecule. As will be appreciated by one of skill in the art, the engineered microbe targeting molecule can be supplied in a lyophilized form or a concentrated form that can diluted prior to use with samples, and it can be supplied in aliquots or in unit doses. Preferred formulations include those that are non-toxic to the cells and/or does not affect growth rate or viability etc. In some embodiments of any of the aspects, the engineered microbe targeting molecule is supplied linked to a solid support. In some embodiments of any of the aspects, the engineered microbe targeting molecule and support are supplied separately, optionally with reagents to link the engineered microbe targeting molecule to the support.

In some embodiments of any of the aspects, the components described herein can be provided singularly or in any combination as a kit. The kit includes the components described herein, e.g., a composition comprising an engineered microbe targeting molecule. Such kits can optionally include one or more agents that permit the detection of at least one microbe or at least one microbe component or a set thereof. In addition, the kit optionally comprises informational material. In some embodiments of any of the aspects, the kits provided herein optionally comprise an aliquot of at least one reagent for sample preparation, including but not limited to a protease, a composition that is more acidic than the than the digested microbe or microbe components, or a matrix solution, as described further herein. The kit can also contain a substrate for coating culture dishes, such as laminin, fibronectin, Poly-L-Lysine, or methylcellulose.

In some embodiments of any of the aspects, the compositions in the kit can be provided in a watertight or gas tight container which in some embodiments is substantially free of other components of the kit. For example, an engineered microbe targeting composition can be supplied in more than one container, e.g., it can be supplied in a container having sufficient reagent for a predetermined number of microbe detection reactions, e.g., 1, 2, 3 or greater. One or more components as described herein can be provided in any form, e.g., liquid, dried or lyophilized form. It is preferred that the components described herein are substantially pure and/or sterile. When the components described herein are provided in a liquid solution, the liquid solution preferably is an aqueous solution, with a sterile aqueous solution being preferred.

The informational material can be descriptive, instructional, marketing or other material that relates to the methods described herein. The informational material of the kits is not limited in its form. In one embodiment, the informational material can include information about production of the engineered microbe targeting molecule, concentration, date of expiration, batch or production site information, and so forth. In one embodiment, the informational material relates to methods for using or administering the components of the kit.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

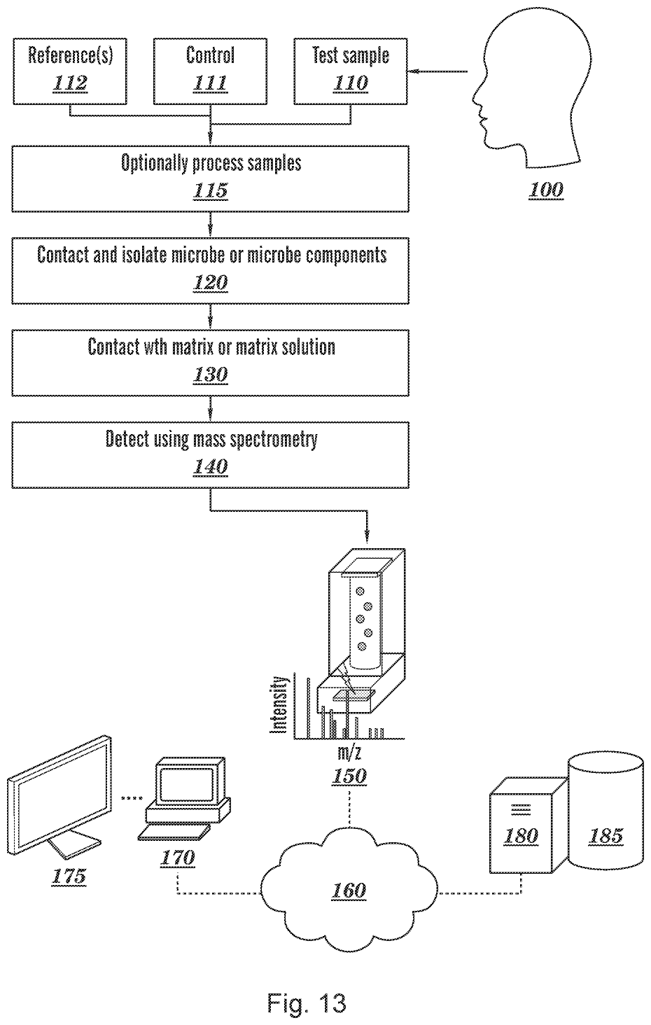

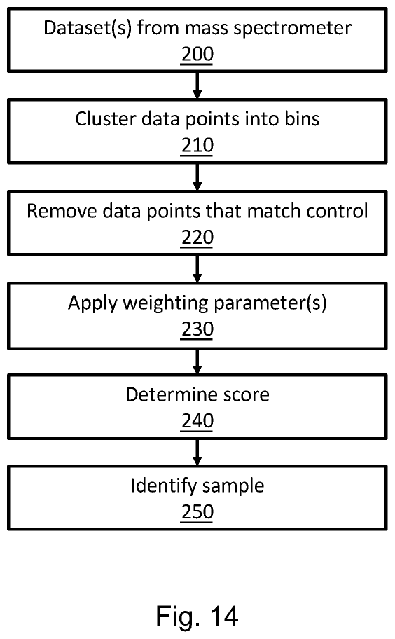

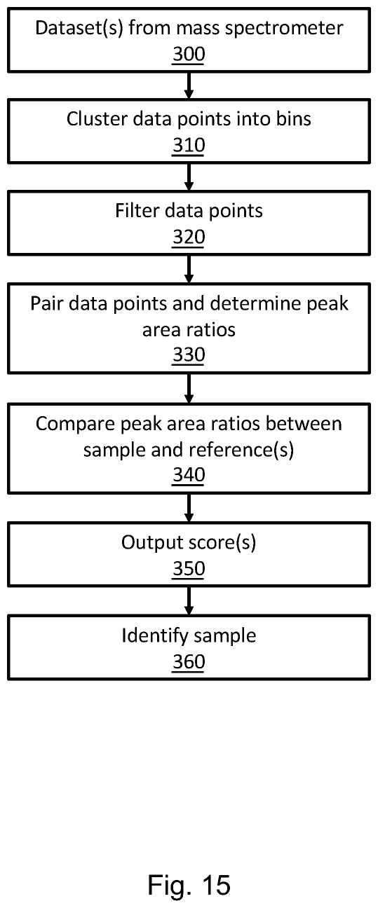

FIG. 13 illustrates an example overview of a system for implementing the current disclosure. The system includes a mass spectrometer 150 into which isolated microbes and microbe components are input. Data output from the mass spectrometer 150 can be input into a program that may be stored in a database 185.

The computing device 170 and server 180 may be connected by a network 160 and the network 160 may be connected to various other devices, servers, or network equipment for implementing the present disclosure. A computing device 170 may be connected to a display 175. Computing device 170 may be any suitable computing device, including a desktop computer, server (including remote servers), mobile device, or other suitable computing device. In some examples, algorithm(s) as described herein and other software may be stored in database 185 and run on server 180. Additionally, mass spectrometer data (e.g., mass spectra) and data processed or produced by said algorithms or programs (e.g., processed profiles, scores, output tables, etc.), may be stored in database 185.

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present technology as disclosed herein, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” (also referred to herein as a processor) encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments of any of the aspects, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments of any of the aspects, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

A level which is less than a reference level can be a level which is less by at least about 10%, at least about 20%, at least about 50%, at least about 60%, at least about 80%, at least about 90%, or less relative to the reference level. In some embodiments of any of the aspects, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

In some embodiments of any of the aspects, the reference can be a level of the target molecule in a population of subjects who do not have or are not diagnosed as having, and/or do not exhibit signs or symptoms of a microbial infection. In some embodiments of any of the aspects, the reference can also be a level of expression of the target molecule in a control sample, a pooled sample of control individuals or a numeric value or range of values based on the same. In some embodiments of any of the aspects, the reference can be the level of a target molecule in a sample obtained from the same subject at an earlier point in time, e.g., the methods described herein can be used to determine if a subject's sensitivity or response to a given therapy is changing over time.

In some embodiments of the foregoing aspects, the expression level of a given gene can be normalized relative to the expression level of one or more reference genes or reference proteins.

In some embodiments of any of the aspects, the reference level can be the level in a sample of similar cell type, sample type, sample processing, and/or obtained from a subject of similar age, sex and other demographic parameters as the sample/subject for which the level of a microbial infection is to be determined. In some embodiments of any of the aspects, the test sample and control reference sample are of the same type, that is, obtained from the same biological source, and comprising the same composition, e.g. the same number and type of cells.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments of any of the aspects, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of microbial infection. A subject can be male or female.

A subject can be one who has been previously diagnosed with or identified as suffering from or having a condition in need of treatment (e.g. for a microbial infection) or one or more complications related to such a condition, and optionally, have already undergone treatment for a microbial infection or the one or more complications related to a microbial infection. Alternatively, a subject can also be one who has not been previously diagnosed as having a microbial infection or one or more complications related to a microbial infection. For example, a subject can be one who exhibits one or more risk factors for a microbial infection or one or more complications related to a microbial infection or a subject who does not exhibit risk factors.

A “subject in need” of treatment for a particular condition can be a subject having that condition, diagnosed as having that condition, or at risk of developing that condition.

The term “effective amount” as used herein refers to the amount of for example an antimicrobial therapeutic agent needed to alleviate at least one or more symptom of the disease or disorder, and relates to a sufficient amount of pharmacological composition to provide the desired effect. The term “therapeutically effective amount” therefore refers to an amount of an antimicrobial therapeutic agent that is sufficient to provide a particular anti-microbial effect (e.g., microbial killing or decreased microbial growth) when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of an antimicrobial therapeutic agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for an antimicrobial therapeutic agent, among others. The dosage can be determined by a physician and adjusted, as necessary, to suit observed effects of the treatment.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments of any of the aspects, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wild-type reference polypeptide's activity according to the assays described below herein. A functional fragment can comprise conservative substitutions of the sequences disclosed herein.

In some embodiments of any of the aspects, the polypeptide described herein can be a variant of a sequence described herein. In some embodiments of any of the aspects, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA, cDNA, microbial DNA. Suitable RNA can include, e.g., mRNA, microbial RNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments of any of the aspects, the expression of a biomarker(s), target(s), or gene/polypeptide described herein is/are tissue-specific. In some embodiments of any of the aspects, the expression of a biomarker(s), target(s), or gene/polypeptide described herein is/are global. In some embodiments of any of the aspects, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from subjects having a microbial infection, as compared to a comparable sample taken from control subjects (e.g., a healthy subject). The term “biomarker” is used interchangeably with the term “marker.”

In some embodiments of any of the aspects, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectrometric, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the engineered microbe targeting molecule described herein is exogenous. In some embodiments of any of the aspects, the engineered microbe targeting molecule described herein is ectopic. In some embodiments of any of the aspects, the engineered microbe targeting molecule described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a microbial infection. The term “treating” includes reducing or alleviating at least one adverse effect or symptom of a condition, disease or disorder associated with a microbial infection. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments of any of the aspects, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments of any of the aspects, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments of any of the aspects, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of detecting a microbe or microbe component, the method comprising the following steps:
   i) contacting a sample with an engineered microbe-targeting molecule linked to a support;
   ii) isolating the microbe or microbe components bound to the engineered microbe-targeting molecule;
   iii) contacting the microbe or microbe components with a matrix or matrix solution on a target substrate; and
   iv) detecting the microbe or microbe components using a mass spectrometric method.
2. A method of detecting a microbial infection in a patient, the method comprising the following steps:
   i) contacting a patient sample with an engineered microbe-targeting molecule linked to a support;
   ii) isolating the microbe or microbe components bound to the engineered microbe-targeting molecule;
   iii) contacting the microbe or microbe components with a matrix or matrix solution on a target substrate; and
   iv) detecting the microbe or microbe components using a mass spectrometric method.
3. The method of any of paragraphs 1-2, wherein the microbe components comprise microbe-associated molecular patterns (MAMPs).
4. The method of any of paragraphs 1-3, wherein the microbe components comprise pathogen-associated molecular patterns (PAMPs).
5. The method of any of paragraphs 1-4, wherein the detecting of step iv outputs mass spectrometric data obtained from the sample as a sample library.
6. The method of any of paragraphs 1-5, wherein the detecting of step iv comprises comparing the sample library with at least one control library of mass spectrometric data.
7. The method of paragraph 6, wherein the at least one control library of mass spectrometric data comprises data obtained from at least one control sample not comprising any known microbes or microbe components.
8. The method of any of paragraphs 1-7, wherein the detecting of step iv comprises comparing the sample library with at least one reference library of mass spectrometric data.
9. The method of paragraph 8, wherein the at least one reference library of mass spectrometric data comprises data obtained from at least one sample comprising a known microbe or components of at least one known microbe.
10. The method of any of paragraphs 1-9, wherein the detecting of step iv comprises analyzing the sample library with a control system comprising one or more processors, the control system configured to execute machine executable code using a clustering process, wherein each cluster comprises a cluster of data points from a single molecular signal of interest.
11. The method of any of paragraphs 10, wherein each cluster of data points is at least 1 Dalton (Da) wide.
12. The method of any of paragraphs 10-11, wherein the cluster of data points is based on m/z peaks identified by the maximum intensity of that cluster.
13. The method of any of paragraphs 10-11, wherein the cluster of data points is based on m/z peaks identified by the mean m/z value of that cluster.
14. The method of any of paragraphs 5-13, wherein the detection process further comprises removing at least one data point of the sample library or the control library, wherein the at least one data point comprises a repeatability value at or below a pre-determined threshold.
15. The method of any of paragraphs 10-14, wherein the clustering process further comprises removing at least one data point of the sample library that matches a data point in a control library within +/−0.3 Da.
16. The method of any of paragraphs 10-15, wherein the clustering process further comprises removing at least one data point of the sample library that does not match a data point in at least one reference library within +/−0.3 Da.
17. The method of any of paragraphs 1-17, wherein the detecting of step iv comprises determining a peak area ratio of at least one pair of data points within the sample library and at least one pair of data points in at least one reference library.
18. The method of paragraph 18, wherein at least one peak area ratio of the sample library is compared to at least one corresponding peak area ratio of at least one reference library.
19. The method of paragraphs 19, wherein a score is calculated based on the comparison of at least one peak area ratio of the sample library and at least one corresponding peak area ratio of at least one reference library.
20. The method of any of paragraphs 10-19, wherein the clustering process further comprises applying a weighting parameter, comprising a frequency weighting parameter and/or a trustworthiness weighting parameter, wherein the weighting parameter identifies the proportion of data points that are unique to the sample library or common with a reference library or a control library.
21. The method of paragraph 20, wherein the frequency weighting parameter increases the weight of a data point if the cluster containing the data point comprises additional other data points.
22. The method of paragraph 20, wherein the trustworthiness weighting parameter decreases the weight of a data point if the data point is found within multiple clusters in the sample library, reference library, and/or control library.
23. The method of any of paragraphs 1-22, further comprising:
   i) assigning a score to the sample based on similarity with a reference library; and/or
   ii) identifying the microbe in the sample as belonging to a reference library based on the score being above a predetermined threshold.
24. The method of any of paragraphs 1-23, further comprising identifying the species of the microbe detected in the sample according to the data points analyzed and outputting said species on a display.
25. The method of any of paragraphs 1-24, further comprising identifying the strain of the microbe detected in the sample according to the data points analyzed and outputting said strain on a display.
26. The method of any of paragraphs 1-25, further comprising determining whether the microbe detected in the sample is sensitive or resistant to an antimicrobial therapeutic according to the data points analyzed and outputting said sensitivity on a display.
27. The method of any of paragraphs 1-26, further comprising assigning the patient to an infection category according to the data points analyzed and outputting the infection category on a display.
28. The method of any of paragraphs 1-27, wherein the results of step iv comprise a profile, wherein said profile indicates the presence or absence of at least one microbe or microbe component.
29. The method of paragraph 28, wherein the profile is specific to at least one microbe or microbe component.
30. The method of any of paragraphs 28-29, wherein the profile comprises a set of data points for a specific microbe or specific set of microbes, and wherein each profile comprises a set of m/z peaks clustered for a single molecular signal of interest.
31. The method of any of paragraphs 28-30, wherein the profile for the specific microbe does not include any of the set of data points associated with a control library.
32. The method of any of paragraphs 28-31, wherein the profile is distinguishable from the profiles of other microbes or microbe components or sets thereof
33. The method of any of paragraphs 28-32, wherein the profile is set forth in any one of FIG. 4A-4B, FIG. 5A-5G, FIG. 8B-8J, or FIG. 9A-9B.
34. The method of any of paragraphs 1-33, wherein the support is a magnetic support.
35. The method of any of paragraphs 1-34, wherein the step of isolating comprises applying a magnet to the sample.
36. The method of any of paragraphs 1-35, wherein the step of isolating comprises washing the support with a buffer to remove unbound cells or biomolecules.
37. The method of any of paragraphs 1-36, wherein the step of isolating further comprises eluting the microbe or microbe components from the support.
38. The method of paragraph 37, wherein the step of eluting comprises heating to a temperature of at least 70° C. and/or shaking at a speed of at least 950 rpm for no longer than 30 minutes.
39. The method of paragraph 38, wherein the heating to a temperature of at least 70° C. is performed in calcium-free water.
40. The method of paragraph 38, wherein the step of eluting comprises treatment with ethylenediaminetetraacetic acid (EDTA).
41. The method of any of paragraphs 1-40, wherein the step of isolating comprises concentrating the microbe or microbe components into a smaller volume from a larger volume of the sample.
42. The method of paragraph 41, wherein the isolated volume is less than the volume of the sample.
43. The method of any of paragraphs 1-42, wherein the target substrate is evenly sprayed with matrix solution prior to step iii to generate a homogenous layer of crystallized matrix on top of the target substrate.
44. The method of any of paragraphs 1-43, wherein the matrix solution comprises a matrix selected from the group consisting of 2′,6′-dihydroxyacetophenone (DHAP), α-Cyano-4-hydroxycinnamic acid (CHCA), sinapic acid (SA), super DHB, 2′,4′,6′-trihydroxyacetophenone monohydrate (THAP), and 9-aminoacridine (9-AA), and the matrix is dissolved in an organic, aqueous solution.
45. The method of any of paragraphs 1-44, wherein the matrix solution is 40 mg/mL 2,5-Dihydroxybenzoic acid (DHB) in 50% methanol, 50% water, 0.1% formic acid.
46. The method of any of paragraphs 1-45, wherein the mass spectrometric method is Matrix-Assisted Laser Desorption Ionization (MALDI-TOF).
47. The method of any of paragraphs 1-46, wherein the mass spectrometric method is automated.
48. The method of any of paragraphs 1-47, wherein the sample comprises blood, serum, plasma, sputum, urine, joint fluid, or any other tissue or biological sample.
49. The method of any of paragraphs 2-48, wherein the patient has been treated with antibiotics.
50. The method of any of paragraphs 1-49, wherein the sample contains at least one antibiotic.
51. The method of any of paragraphs 1-50, wherein the sample contains at least two antibiotics.
52. The method of any of paragraphs 2-51, wherein the patient has been treated with antifungals.
53. The method of any of paragraphs 1-52, wherein the sample contains antifungals.
54. The method of any of paragraphs 2-53, wherein the patient has been treated with antivirals.
55. The method of any of paragraphs 1-54, wherein the sample contains antivirals.
56. The method of any of paragraphs 1-55, wherein the sample has not been cultured.
57. The method of any of paragraphs 1-56, wherein the time from the step of collecting the sample to the end of detecting takes equal to or less than 90 minutes.
58. The method of any of paragraphs 1-57, wherein the engineered microbe-targeting molecule comprises a microbe surface-binding domain.
59. The method of any of paragraphs 1-58, wherein the microbe surface-binding domain comprises a mannose-binding lectin (MBL).
60. The method of any of paragraphs 1-59, wherein the microbe surface-binding domain comprises a human mannose-binding lectin (MBL).
61. The method of any of paragraphs 1-60, wherein the microbe surface-binding domain comprises a carbohydrate recognition domain (CRD) of MBL.
62. The method of paragraph 61, wherein the CRD is linked to an immunoglobulin or fragment thereof
63. The method of any of paragraphs 61-62, wherein the CRD is linked to an Fc component of human IgG1 (FcMBL).
64. The method of paragraph 34, wherein the magnetic support is a superparamagnetic support.
65. The method of paragraph 34, wherein the magnetic support comprises a magnetic bead, a superparamagnetic bead, or a magnetic microbead.
66. The method of any of paragraphs 1-65, wherein the engineered microbe-targeting molecule comprises FcMBL streptavidin linked to superparamagnetic beads.
67. The method of any of paragraphs 1-66, wherein the engineered microbe-targeting molecule is linked to an ELISA plate.
68. The method of any of paragraphs 1-67, wherein the microbe comprises a Gram-positive bacterial species, a Gram-negative bacterial species, a mycobacterium, a fungus, a parasite, or a virus.
69. The method of any of paragraphs 1-68, wherein the microbial component comprises a component from a Gram-positive bacterial species, a Gram-negative bacterial species, a mycobacterium, a fungus, a parasite, or a virus.
70. The method of paragraph 69, wherein the Gram-positive bacterial species comprises bacteria from the class Bacilli.
71. The method of paragraph 69, wherein the Gram-negative bacterial species comprises bacteria from the class Gammaproteobacteria.
72. The method of paragraph 69, wherein the mycobacterium comprises bacteria from the class Actinobacteria.
73. The method of paragraph 69, wherein the fungus comprises fungus from the class Saccharomycetes.
74. The method of any of paragraphs 1-73, wherein the microbe is selected from the group consisting of Staphylococcus aureus, Streptococcus pyogenes, Klebsiella pneumoniae, Pseudomonas aeruginosa, Mycobacterium tuberculosis, Candida albicans, or Escherichia coli.
75. The method of any of paragraphs 1-74, wherein the microbe is a human pathogen.
76. The method of any of paragraphs 1-75, wherein the sample contains at least one pathogen.
77. The method of any of paragraphs 1-76, wherein the sample contains more than one pathogen.
78. The method of any of paragraphs 1-77, wherein the species of the pathogen is identified.
79. The method of any of paragraphs 1-78, wherein the strain of the pathogen is identified.
80. The method of any of paragraphs 1-79, wherein the drug sensitivity of the pathogen is identified.
81. The method of any of paragraphs 1-80, further comprising providing a therapy model to the patient based on the infection category assigned to the patient.
82. The method of any of paragraphs 1-81, further comprising providing a therapy model to the patient based on the identified pathogen assigned to the patient.
83. The method of any of paragraphs 81-82, wherein the therapy model comprises treatment with a therapeutic agent specific to the pathogen.

Some embodiments of the technology described herein can be defined according to any of the following numbered paragraphs:
1. A method of detecting a microbe or microbe component, the method comprising the following steps:
   i) contacting a sample with an engineered microbe-targeting molecule linked to a support;
   ii) isolating the microbe or microbe components bound to the engineered microbe-targeting molecule;
   iii) contacting the microbe or microbe components with a matrix or matrix solution on a target substrate; and
   iv) detecting the microbe or microbe components using a mass spectrometric method.
2. A method of detecting a microbial infection in a patient, the method comprising the following steps:
   i) contacting a patient sample with an engineered microbe-targeting molecule linked to a support;
   ii) isolating the microbe or microbe components bound to the engineered microbe-targeting molecule;
   iii) contacting the microbe or microbe components with a matrix or matrix solution on a target substrate; and
   iv) detecting the microbe or microbe components using a mass spectrometric method.
3. The method of any of paragraphs 1-2, wherein the microbe components comprise microbe-associated molecular patterns (MAMPs).
4. The method of any of paragraphs 1-3, wherein the microbe components comprise pathogen-associated molecular patterns (PAMPs).
5. The method of any of paragraphs 1-4, wherein the detecting of step iv outputs mass spectrometric data obtained from the sample as a sample library.
6. The method of any of paragraphs 1-5, wherein the detecting of step iv comprises comparing the sample library with at least one control library of mass spectrometric data.
7. The method of paragraph 6, wherein the at least one control library of mass spectrometric data comprises data obtained from at least one control sample not comprising any known microbes or microbe components.
8. The method of any of paragraphs 1-7, wherein the detecting of step iv comprises comparing the sample library with at least one reference library of mass spectrometric data.
9. The method of paragraph 8, wherein the at least one reference library of mass spectrometric data comprises data obtained from at least one sample comprising a known microbe or components of at least one known microbe.
10. The method of any of paragraphs 1-9, wherein the detecting of step iv comprises analyzing the sample library with a control system comprising one or more processors, the control system configured to execute machine executable code using a clustering process, wherein each cluster comprises a cluster of data points from a single molecular signal of interest.
11. The method of paragraph 10, wherein each cluster of data points is at least 1 Dalton (Da) wide.
12. The method of any of paragraphs 10-11, wherein the cluster of data points is based on m/z peaks identified by the maximum intensity of that cluster.
13. The method of any of paragraphs 10-11, wherein the cluster of data points is based on m/z peaks identified by the mean m/z value of that cluster.
14. The method of any of paragraphs 5-13, wherein the detection process further comprises removing at least one data point of the sample library or the control library, wherein the at least one data point comprises a repeatability value at or below a pre-determined threshold.
15. The method of any of paragraphs 10-14, wherein the clustering process further comprises removing at least one data point of the sample library that matches a data point in a control library within +/−0.3 Da.
16. The method of any of paragraphs 10-15, wherein the clustering process further comprises removing at least one data point of the sample library that does not match a data point in at least one reference library within +/−0.3 Da.
17. The method of any of paragraphs 1-16, wherein the detecting of step iv comprises determining a peak area ratio of at least one pair of data points within the sample library and at least one pair of data points in at least one reference library.
18. The method of paragraph 17, wherein at least one peak area ratio of the sample library is compared to at least one corresponding peak area ratio of at least one reference library.
19. The method of paragraphs 18, wherein a score is calculated based on the comparison of at least one peak area ratio of the sample library and at least one corresponding peak area ratio of at least one reference library.
20. The method of any of paragraphs 10-19, wherein the clustering process further comprises applying a weighting parameter, comprising a frequency weighting parameter and/or a trustworthiness weighting parameter, wherein the weighting parameter identifies the proportion of data points that are unique to the sample library or common with a reference library or a control library.
21. The method of paragraph 20, wherein the frequency weighting parameter increases the weight of a data point if the cluster containing the data point comprises additional other data points.
22. The method of paragraph 20, wherein the trustworthiness weighting parameter decreases the weight of a data point if the data point is found within multiple clusters in the sample library, reference library, and/or control library.
23. The method of any of paragraphs 1-22, further comprising:
   i) assigning a score to the sample based on similarity with a reference library; and/or
   ii) identifying the microbe in the sample as belonging to a reference library based on the score being above a predetermined threshold.
24. The method of any of paragraphs 1-23, further comprising identifying the species of the microbe detected in the sample according to the data points analyzed and outputting said species on a display.
25. The method of any of paragraphs 1-24, further comprising identifying the strain of the microbe detected in the sample according to the data points analyzed and outputting said strain on a display.
26. The method of any of paragraphs 1-25, further comprising determining whether the microbe detected in the sample is sensitive or resistant to an antimicrobial therapeutic according to the data points analyzed and outputting said sensitivity on a display.
27. The method of any of paragraphs 1-26, further comprising assigning the patient to an infection category according to the data points analyzed and outputting the infection category on a display.
28. The method of any of paragraphs 1-27, wherein the results of step iv comprise a profile, wherein said profile indicates the presence or absence of at least one microbe or microbe component.
29. The method of paragraph 28, wherein the profile is specific to at least one microbe or microbe component.
30. The method of any of paragraphs 28-29, wherein the profile comprises a set of data points for a specific microbe or specific set of microbes, and wherein each profile comprises a set of m/z peaks clustered for a single molecular signal of interest.
31. The method of any of paragraphs 28-30, wherein the profile for the specific microbe does not include any of the set of data points associated with a control library.
32. The method of any of paragraphs 28-31, wherein the profile is distinguishable from the profiles of other microbes or microbe components or sets thereof
33. The method of any of paragraphs 28-32, wherein the profile is set forth in any one of FIG. 4A-4B, FIG. 5A-5G, FIG. 8B-8J, or FIG. 9A-9B.
34. The method of any of paragraphs 1-33, wherein the support is a magnetic support.
35. The method of any of paragraphs 1-34, wherein the support is a non-magnetic support.
36. The method of any of paragraphs 1-35, wherein the support is a non-magnetic nanoparticle.
37. The method of any of paragraphs 1-36, wherein the support is a mesoporous nanoparticle.
38. The method of any of paragraphs 1-37, wherein the support is mesoporous silica.
39. The method of any of paragraphs 1-38, wherein the step of isolating comprises applying a magnet to the sample.
40. The method of any of paragraphs 1-39, wherein the step of isolating comprises washing the support with a buffer to remove unbound cells or biomolecules.
41. The method of any of paragraphs 1-40, wherein the step of isolating further comprises eluting the microbe or microbe components from the support.
42. The method of paragraph 41, wherein the step of eluting comprises heating to a temperature of at least 70° C. and/or shaking at a speed of at least 950 rpm for no longer than 30 minutes.
43. The method of paragraph 42, wherein the heating to a temperature of at least 70° C. is performed in calcium-free water.
44. The method of paragraph 43, wherein the step of eluting comprises treatment with ethylenediaminetetraacetic acid (EDTA).
45. The method of any of paragraphs 1-44, wherein the step of isolating does not comprise eluting the microbe or microbe components from the support.
46. The method of any of paragraphs 1-45, wherein the step of isolating comprises concentrating the microbe or microbe components into a smaller volume from a larger volume of the sample.
47. The method of paragraph 46, wherein the isolated volume is less than the volume of the sample.
48. The method of any of paragraphs 1-47, wherein the target substrate is evenly sprayed with matrix solution prior to step iii to generate a homogenous layer of crystallized matrix on top of the target substrate.
49. The method of any of paragraphs 1-48, wherein the matrix solution comprises a matrix selected from the group consisting of 2′,6′-dihydroxyacetophenone (DHAP), α-Cyano-4-hydroxycinnamic acid (CHCA), sinapic acid (SA), super DHB, 2′,4′,6′-trihydroxyacetophenone monohydrate (THAP), and 9-aminoacridine (9-AA), and the matrix is dissolved in an organic, aqueous solution.
50. The method of any of paragraphs 1-49, wherein the matrix solution is 40 mg/mL 2,5-Dihydroxybenzoic acid (DHB) in 50% methanol, 50% water, 0.1% formic acid.
51. The method of any of paragraphs 1-50, wherein the mass spectrometric method is Matrix-Assisted Laser Desorption Ionization (MALDI-TOF).
52. The method of any of paragraphs 1-51, wherein the mass spectrometric method is automated.
53. The method of any of paragraphs 1-52, wherein the sample comprises blood, serum, plasma, sputum, urine, joint fluid, or any other tissue or biological sample.
54. The method of any of paragraphs 2-53, wherein the patient has been treated with antibiotics.
55. The method of any of paragraphs 1-54, wherein the sample contains at least one antibiotic.
56. The method of any of paragraphs 1-55, wherein the sample contains at least two antibiotics.
57. The method of any of paragraphs 2-56, wherein the patient has been treated with antifungals.
58. The method of any of paragraphs 1-57, wherein the sample contains antifungals.
59. The method of any of paragraphs 2-58, wherein the patient has been treated with antivirals.
60. The method of any of paragraphs 1-59, wherein the sample contains antivirals.
61. The method of any of paragraphs 1-60, wherein the sample has not been cultured.
62. The method of any of paragraphs 1-61, wherein the time from the step of collecting the sample to the end of detecting takes equal to or less than 90 minutes.
63. The method of any of paragraphs 1-62, wherein the engineered microbe-targeting molecule comprises a microbe surface-binding domain.
64. The method of any of paragraphs 1-63, wherein the microbe surface-binding domain comprises a mannose-binding lectin (MBL).
65. The method of any of paragraphs 1-64, wherein the microbe surface-binding domain comprises a human mannose-binding lectin (MBL).
66. The method of any of paragraphs 1-65, wherein the microbe surface-binding domain comprises a carbohydrate recognition domain (CRD) of MBL.
67. The method of paragraph 66, wherein the CRD is linked to an immunoglobulin or fragment thereof
68. The method of any of paragraphs 66-67, wherein the CRD is linked to an Fc component of human IgG1 (FcMBL).
69. The method of paragraph 68, wherein the magnetic support is a superparamagnetic support.
70. The method of paragraph 68, wherein the magnetic support comprises a magnetic bead, a superparamagnetic bead, or a magnetic microbead.
71. The method of any of paragraphs 1-70, wherein the engineered microbe-targeting molecule comprises FcMBL streptavidin linked to superparamagnetic beads.
72. The method of any of paragraphs 1-71, wherein the engineered microbe-targeting molecule comprises FcMBL linked to mesoporous silica particles.
73. The method of any of paragraphs 1-72, wherein the engineered microbe-targeting molecule is
linked to an ELISA plate. 74. The method of any of paragraphs 1-73, wherein the microbe comprises a Gram-positive bacterial species, a Gram-negative bacterial species, a mycobacterium, a fungus, a parasite, or a virus.
75. The method of any of paragraphs 1-74, wherein the microbial component comprises a component from a Gram-positive bacterial species, a Gram-negative bacterial species, a mycobacterium, a fungus, a parasite, or a virus.
76. The method of paragraph 75, wherein the virus is a coronavirus.
77. The method of paragraph 75, wherein the virus is a severe acute respiratory syndrome coronavirus 2 (SARS-CoV-2).
78. The method of paragraph 75, wherein the Gram-positive bacterial species comprises bacteria from the class Bacilli.
79. The method of paragraph 75, wherein the Gram-negative bacterial species comprises bacteria from the class Gammaproteobacteria.
80. The method of paragraph 75, wherein the mycobacterium comprises bacteria from the class Actinobacteria.
81. The method of paragraph 75, wherein the fungus comprises fungus from the class Saccharomycetes.
82. The method of any of paragraphs 1-81, wherein the microbe is selected from the group consisting of Staphylococcus aureus, Streptococcus pyogenes, Klebsiella pneumoniae, Pseudomonas aeruginosa, Mycobacterium tuberculosis, Candida albicans, or Escherichia coli.
83. The method of any of paragraphs 1-82, wherein the microbe is a human pathogen.
84. The method of any of paragraphs 1-83, wherein the sample contains at least one pathogen.
85. The method of any of paragraphs 1-84, wherein the sample contains more than one pathogen.
86. The method of any of paragraphs 1-85, wherein the species of the pathogen is identified.
87. The method of any of paragraphs 1-86, wherein the strain of the pathogen is identified.
88. The method of any of paragraphs 1-87, wherein the drug sensitivity of the pathogen is identified.
89. The method of any of paragraphs 1-88, further comprising providing a therapy model to the patient based on the infection category assigned to the patient.
90. The method of any of paragraphs 1-89, further comprising providing a therapy model to the patient based on the identified pathogen assigned to the patient.
91. The method of any of paragraphs 1-90, wherein the therapy model comprises treatment with a therapeutic agent specific to the pathogen.

### EXAMPLES

### Example 1

Application of MALDI-TOF Mass Spectrometry for the Rapid Identification of PAMPs in Blood Culture Negative Septic Patients Using FcMBL-Coated Magnetic Beads

Described herein are methods using superparamagnetic beads which have been streptavidin linked with an engineered version of the naturally occurring human opsonin Mannose Binding Lectin (MBL). This protein comprises the carbohydrate recognition domain of MBL linked with the Fc component of human IgG1, and is known as FcMBL (see e.g., Kang et al., 2014, Nat. Med. 20 (10), 1211-1216; Cartwright et al., 2016, EBioMedicine 9, 217-227; Didar et al., 2015, Biomaterials 67, 387-392; Dommett et al., 2006, HLA 68 (3), 193-209; Seiler et al., 2019 Jan. 25, F1000Res. 8:108; each of which is incorporated by reference herein in its entirety). FcMBL binds to a wide variety of microbial cell wall and membrane components known as pathogen-associated molecular patterns (PAMPs) including lipopolysaccharide (LPS) endotoxin, lipoteichoic acid, and attached or released outer membrane vesicles, among others.

Immunomagnetic separation (i.e., an antibody linked to a magnetic bead) has been applied previously as a method of extracting and concentrating low-abundance live microorganisms from complex biological fluids to be analyzed via MALDI-TOF MS (see e.g., Ho and Reddy, 2010, Clin Chem. 56(4), 525-536; Madonna et al., 2003, Rapid Commun Mass Spectrom. 17(3): 257-263; each of which is incorporated by reference herein in its entirety). One key advantage to using FcMBL beads is the ability to capture and concentrate fragmented microbial components in addition to live, whole cells, thus increasing the sensitivity of FcMBL methods. FcMBL beads are capable of binding more than 190 different isolates from over 95 different bacterial, viral, fungal, and parasitic species directly from infected blood, thus bypassing the cultivation step and improving upon current diagnostic methods (see e.g., Seiler et al., 2019, supra). PAMPs can be quantified directly from whole blood using a modified ELISA method that was developed for use with FcMBL beads called the Enzyme-Linked Lectin-Sorbent Assay (ELLecSA). This assay has been developed as a broad-spectrum infection diagnostic capable of classifying sepsis in blood culture positive and blood culture negative clinical samples with a diagnostic accuracy of 87% (18).

Described herein is a diagnostic tool which provides rapid pathogen identification within the framework of standard-of-care antibiotic use by combining the capture of PAMPs using FcMBL-coated magnetic beads with MALDI-TOF MS fingerprint identification. Described herein is a method of extracting and concentrating PAMPs captured directly out of spiked whole blood using FcMBL-coated beads. The captured material was used to create unique genera-specific fingerprint libraries built from the m/z spectra produced by MALDI-TOF MS. This information was applied to develop a proprietary clustering algorithm and probabilistic model capable of identifying infection from blood spiked with as little as 10 CFU/mL equivalent of unknown antibiotic-treated pathogen. This system correctly identified clinical samples from 7 out of 13 (53.84%) septic patients at the genus level and 10/13 (76.92%) at the group level in only 90 minutes from blood draw. This diagnostic system can be applied as a rapid, culture-free diagnostic tool for characterizing infection in septic patients for whom current diagnostics are limited.

### Microbiology Methods

One species from 7 genera were selected for development of reference pathogen fingerprint libraries: 3 Gram-negative bacteria Pseudomonas aeruginosa (internal strain ID: Crimson 41504), Klebsiella pneumoniae (internal strain ID: Boston Children's Hospital 631), and Escherichia coli (internal strain ID: Crimson 41949), 2 Gram-positive bacteria Staphylococcus aureus (internal strain ID: Crimson 3518) and Streptococcus pyogenes (internal strain ID: Crimson 011014), a fungus Candida albicans (internal strain ID: Hospital Joseph-Ducuing 1311), and a mycobacterium, Mycobacterium tuberculosis (BEI Resources H37Rv).

Isolates were first cultured overnight on 5% sheep blood agar in 5% CO₂ at 37° C. Liquid cultures were prepared by the addition of a fresh colony into RPMI+10 mM glucose and growing the pathogen to log phase (0.5 McFarland). An appropriate antibiotic or antibiotic combination was administered overnight and cellular death was confirmed by plating. Gram-negative bacteria were treated with cefepime (1 mg/mL) and amikacin (0.5 mg/mL). Gram-positive bacteria were treated with vancomycin (0.5 mg/mL). C. albicans was treated with amphotericin B (40 μg/mL). Due to high levels of LPS, E. coli cultures were centrifuged at 3000 rpm at 4° C. for 8 minutes, the supernatant was discarded, and the resulting pellet was re-suspended in half the volume of RPMI+10 mM glucose. Note that M. tuberculosis samples were not cultured and were instead purchased as inactivated via 2.4 mRads of gamma irradiation rather than antibiotics. M. tuberculosis cell lysate was sonicated for 3 minutes prior to sample preparation.

### Sample Preparation

A capture solution containing 7.3 mL of tris-buffered saline-tween 20+5 mM calcium (TBST Ca²⁺), 500 μl of 1M heparin, 100 μl of 1M glucose, 125 μl of FcMBL beads in 1% BSA/PBS pH 7.0+10 mM EDTA, 1.8 mL healthy donor whole blood, and 200 μl of microbial lysate was mixed on a HulaMixer™ for 30 minutes. For pathogen-free control samples, the capture solution was prepared with 2 mL of blood and without the addition of microbial lysate. The beads carrying the captured PAMPs were isolated and concentrated into a 2 mL Eppendorf tube using a neodymium magnet tube rack. The beads were then washed 3 times in 2 mL of tris-buffered-saline+5 mM octyl-13-D-glucopyranoside+5 mM calcium (TBSG CO followed by a final wash in 2 mL of HPLC-grade H₂O. The beads were then re-suspended in 125 μl of HPLC-grade H₂O. The captured PAMPs were eluted off of the beads by subjecting the solution to 70° C. heat for 30 minutes while mixing at 950 rpm on a bench top shaker. The supernatant containing the eluted material was separated from the beads using a neodymium magnet tube rack and then removed and stored at −80° C.

### Enzyme-Linked Lectin-Sorbent Assay (ELLecSA)

A modified ELLecSA assay was performed to quantify the amount of PAMPs found in each sample. This procedure is as outlined previously with one minor modification (Cartwright et al., 2016, supra). During the capture step, each well contained 10 μl of sample, 890 μl of TBST Ca²⁺, and 100 μl of FcMBL bead solution. This modification was made due to the high concentration of PAMPs found in each sample and to preserve sample volume for mass spectrometry analysis.

### MALDI-TOF MS

Matrix was prepared by addition of 10 μg of 2,5-dihydroxybenzoic acid (DHB) to 500 μl of TA30 (30:70 acetonitrile:1% trifluoroacetic acid (TFA) in water). The matrix was layered underneath the experimental sample on a ground steel MS plate. All spots were analyzed using the Bruker Ultraflextreme MALDI-TOF MS. Sample spots were analyzed using both reflector positive (RP) mode, with a m/z ratio range of 700-5,000 Da, and linear positive (LP) mode with a m/z ratio range of 4,000-20,000 Da. M. tuberculosis-specific peaks could only be visualized in RP mode, whereas E. coli-specific peaks could only be visualized in LP mode. All other species could be visualized in both modes. Each pathogen fingerprint reference library was built from 5 independent sample preparations which were used to produce 50 spectra in both modes, where applicable.

### Algorithm and Spectrum Analysis

The proprietary algorithm was designed to analyze pathogen fingerprint libraries by accomplishing two tasks. First, the m/z peaks from all spectra were clustered into bins, where each bin was defined as the collection of peaks corresponding to a single molecule of interest and was identified by the mean m/z value of that bin. All m/z bins found to be common with the control library were removed from each pathogen fingerprint library. Second, the algorithm was designed to predict the identity of an unknown sample by identifying the proportion of m/z peaks that were unique to the unknown sample and those that were common with exactly one of the known n pathogen libraries or the control library. By keeping a count of these n+2 cluster types and normalizing them, the probability that the unknown sample corresponds to one of the known pathogen libraries can be calculated. This estimate is “frequency weighted”, in other words, the greater the number of peaks that occupy a cluster, the more often the corresponding molecule is encountered during mass spectrometry, and the greater it contributes to the probability estimate. Additionally, another weighting parameter called “trustworthiness” was introduced that indicates how much the spectra can be trusted by providing a likelihood of spuriousness of an m/z peak, given that the cluster it belongs to is occupied by peaks from two libraries. As an example, considering a cluster of peaks from two libraries, a well-balanced cluster proportion (e.g., approximately 50:50) is more likely to indicate peaks belonging to both libraries, while a lopsided cluster proportion (e.g., 10:90 or 90:10) is more likely to indicate peaks belonging to the single dominating library. Therefore, weighting both by cluster frequencies and cluster proportions safeguards the algorithm from noisy spectra. As shown herein, all unknown sample spectra were frequency weighted and analyzed, where cluster proportions from 15:85 to 85:15 are considered trustworthy. Moreover, obtaining a point estimate of the probability is not entirely satisfactory, especially if there are very few peaks in the spectra in the first place. To that end, p-value adjusted thresholds that a probability estimate must satisfy in order to be deemed significant at a certain confidence level can be defined. As an example, if there are n libraries, 1 of which is the correct identification of the unknown sample, and k clusters, then one can treat every cluster contribution to the correct library as a “success” and to all others as “failures”, leading to a binomial model with k trials and probability of success 1/n. If α is the significance level, then the quantile function of this binomial distribution with target probability (1-α), divided by n, returns the adjusted threshold that the probability estimate must satisfy. (Note that α=0.5 corresponds to a threshold of 1/n, which is the threshold to perform better than a random guess.) This algorithm was adapted into a user-friendly application format. A detailed schematic of the algorithm is summarized in FIG. 6 and FIG. 7A-7C.

### Unknown Sample Identification

A blinded study was conducted to identify unknown spiked blood sample preparations. An individual to whom the sample identification was unknown collected a total of 5 spectra in the desired m/z range from each sample. The unknown spectra were input into the algorithm where it was compared with that of the existing pathogen and control libraries, and an identity prediction was made. The species prediction with the highest assigned probability value that lies above the p-value adjusted threshold (where α=0.005) was selected as the identification output. If the species prediction with the highest assigned probability was less than the p-value adjusted threshold, the prediction was considered insignificant (which can be denoted herein by the asterisk symbol: *; e.g., as shown in Tables 2 and 4).

### Clinical Sample Collection

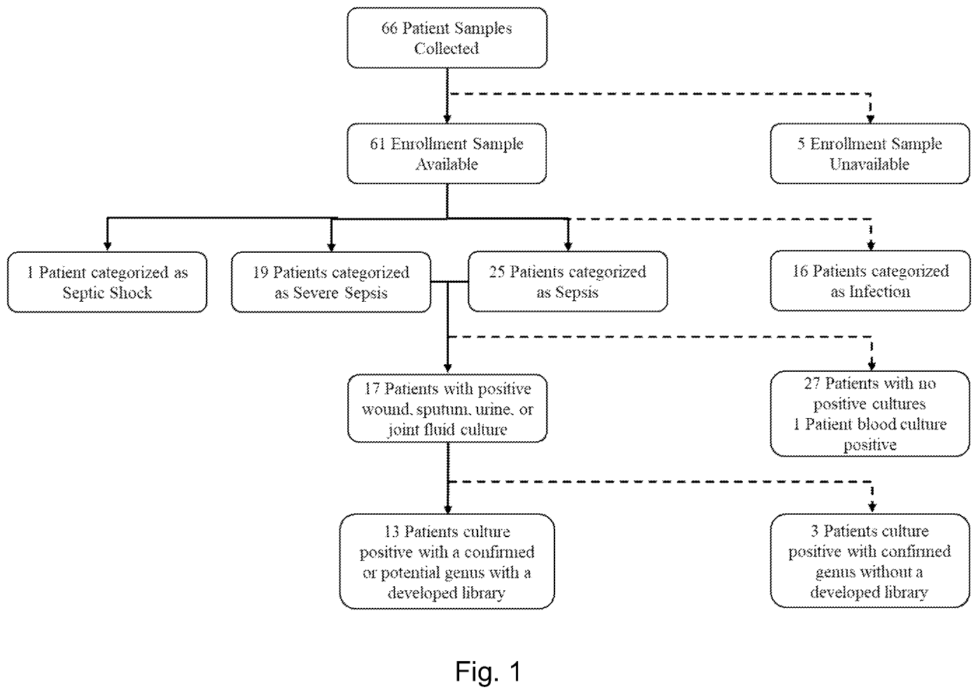

To assess the capabilities of the system as a diagnostic tool in the clinic, blood samples collected from patients with suspected septic infection were analyzed. This data was collected as a part of a secondary analysis to a multicenter study of early detection of inflammatory biomarkers in infection, which collected patient blood samples from four regional emergency departments. The analysis included collection and sample preparation from patients within the suspected infection study arm. The inclusion criteria for patients with suspected infection were the following: 1) the patient is at least 18 years old, 2) the patient has a clinically confirmed or suspected infection as determined by the emergency department, 3) the patient is set to be admitted to the hospital, and 4) the patient has been in the hospital for 24 hours or less. In order to validate whether the diagnostic system could accurately identify the infection within the constraints of the number of developed pathogen fingerprint libraries, the following criteria for patient samples to be analyzed were set as follows: 1) an at-enrollment sample (time point: 0 hr) has been prepared; 2) the patient has been categorized within sepsis (infection+2 or more systemic inflammatory response syndrome (SIRS) criteria), severe sepsis (infection+1 or more organ dysfunctions), or septic shock (infection+1 or more hypoperfusion criteria); 3) the patient is blood culture negative but has a positive secondary culture (wound, sputum, urine, or joint fluid) to validate species identity; and 4) the positive culture must be identified as a confirmed or potential genus for which a library has been developed. The SIRS criteria included: temperature >100.4° F. or <96.8° F., heart rate ≥90 beats/minute, respiratory rate >20 breaths/minute or saturation <90% on room air or PaCO₂≤32 mm Hg or the use of mechanical ventilation, and white blood cell count ≥12,000 or ≤4,000 cells/μL or >10% bands. The patient sample selection for the analysis is summarized in FIG. 1.

FcMBL Bead Capture and Concentration of PAMPs from Whole Blood

FcMBL-coated magnetic beads were used to prepare samples by capture of PAMPs from 7 clinically relevant pathogens (S. aureus, S. pyogenes, K. pneumoniae, P. aeruginosa, E. coli, C. albicans, and M. tuberculosis) which were fragmented by administration of appropriate antibiotic(s). These 7 pathogens were chosen to demonstrate the ability of the system to identify a diverse set of pathogen genera. All selected, apart from M. tuberculosis, are within the top most predominant causes of septic infection. A proof-of-concept study was first conducted with samples prepared in buffer to validate the capacity of the system to develop robust fingerprint libraries to be used as a reference for positive microbial identification (see e.g., FIG. 8A-8J). These results established this method as a diagnostic tool and authorized sample preparation in pathogen-spiked blood.

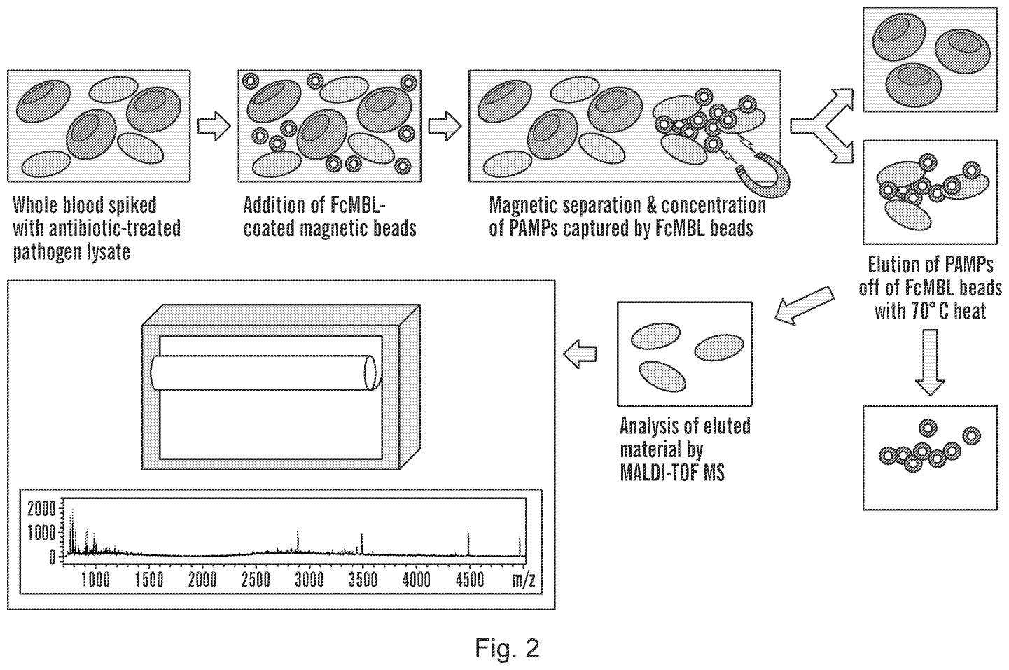

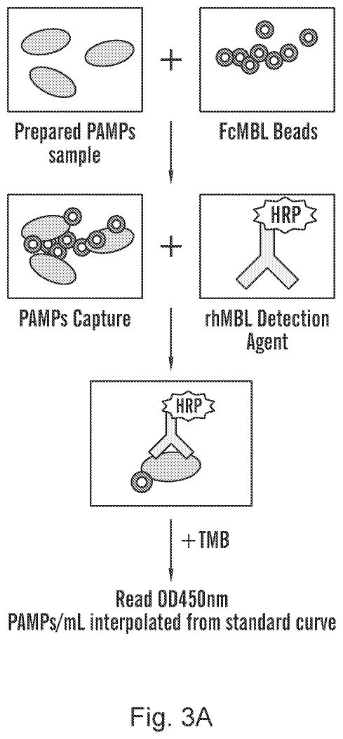

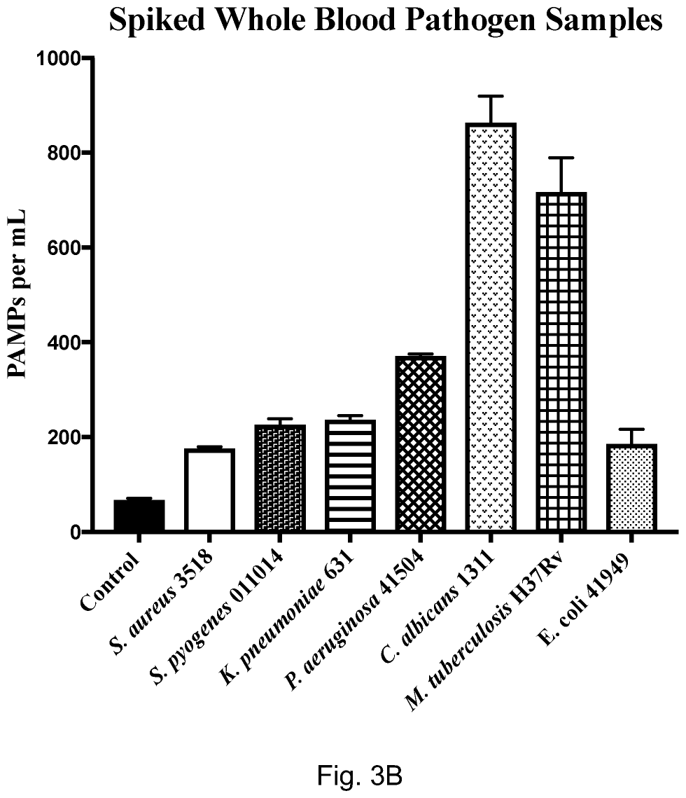

Samples were prepared for mass spectrometry analysis by the addition of FcMBL-coated beads to whole blood spiked with 1×10⁸ CFU/mL equivalent of pathogen lysate. The PAMPs captured by the beads were concentrated onto a magnet, washed, and eluted off with heat (see e.g., FIG. 2). The samples were then quantified by ELLecSA to determine the concentration of eluted PAMPs present for MALDI-TOF MS analysis (see e.g., FIG. 3A). Pathogen samples were found to contain 2 to 8-fold greater concentration of PAMPs/mL than pathogen-free controls (see e.g., FIG. 3B). Due to the differences in composition of microbial lysate and FcMBL binding affinities, the concentration of collected PAMPs varied between species. As demonstrated herein, FcMBL beads can rapidly capture and isolate high concentrations of PAMPs directly out of whole blood.

### Development of Genera-Specific Reproducible Pathogen Fingerprint Libraries

Sample analysis was conducted using the Bruker Ultraflextreme mass spectrometer using 2,5-dihydroxybenzoic acid matrix. Preliminary analysis of pathogen samples prepared in buffer were conducted to rapidly evaluate the optimal MALDI-TOF MS mode and range of analysis for each pathogen when spiked blood, where increased background may obscure observation of pathogen-specific peaks by visual appearance alone (see e.g., FIG. 8A-8B). In buffer, changing the class of antibiotic administered only modestly altered the resulting spectra in both a Gram-negative and Gram-positive species (see e.g., Table 2, Table 5, FIG. 9A-9B). S. aureus, S. pyogenes, K. pneumoniae, P. aeruginosa, C. albicans, and M. tuberculosis can be visualized using reflector positive mode with an m/z range of 700-5,000 Da (see e.g., FIG. 4A). E. coli samples cannot be visualized within this size range. However, E. coli can be visualized when using linear positive mode with an m/z range of 4,000-20,000 Da (see e.g., FIG. 4B). All other species can also be evaluated in this mode except forts. tuberculosis. To develop libraries sufficient to be applied to identify any one of 7 species in blood, a collective library from both size ranges was established for all organisms, where applicable. Some noticeable variation among organisms can be visually observed in respect to individual peak m/z values and the total number of peaks in both size ranges, however algorithmic analysis is necessary to evaluate the unique nature of each species' spectra and its reproducibility across independent sample preparations.

The collected spectra of each species were assembled into pathogen fingerprint libraries using the following workflow: five independent sample preparations were prepared for each species spiked into whole blood, then 10 spectra were collected from each sample preparation in both MALDI-TOF MS modes (where applicable) to assemble a total of 50 spectra in the appropriate size range. Each of the pathogen fingerprint libraries were input into the algorithm, which organized all the spectra by clustering the m/z peaks into bins, where each bin corresponded to a single molecule of interest and all bins found to be associated with the control library were removed. The percent frequency of each bin within the resulting fingerprint library was calculated to evaluate the reproducibility of all spectra contained within the total library (see e.g., FIG. 5A-5G). There was a sufficient amount of high frequency, pathogen-specific peaks found for all fingerprint libraries, although it varied between species. Each of the fingerprint libraries contained 5 to 28 peaks occurring in at least 70% of the total spectra. P. aeruginosa and K. pneumoniae had the greatest number of high frequency peaks, totaling 19 and 28 peaks appearing in at least 70% of the spectra respectively, whereas C. albicans and M. tuberculosis were on the lower end totaling 5 peaks of that frequency each. Collectively, this system produced robust pathogen fingerprint libraries for 7 relevant species spiked into whole blood.

TABLE 1
Baseline Patient Characteristics

| | Patients with Suspected Infection |
|---|---|
| Number of patients | 13 |
| Age | 60.35 ± 17.71 |
| Female gender (%) | 57.14 |
| Sepsis (%) | 46.15 |
| Severe sepsis (%) | 46.15 |
| Septic shock (%) | 7.69 |
| SOFA score | 1.54 ± 1.22 |
| Mortality (%) | 7.69 |

Clinical blood samples were collected from 13 patients from four emergency departments as a part of a secondary analysis multicenter study of early detection of inflammatory biomarkers in infection. The baseline characteristics for these patients are as outlined above in Table 1.

### Positive Identification of Unknown Pathogen Samples at Clinically Relevant Infection Levels

To evaluate whether the developed pathogen fingerprint libraries were sufficient to be applied to correctly identify a species, a blinded study was conducted to identify unknown spiked blood samples. An individual to whom the sample identity was unknown collected 5 spectra for each sample and input the corresponding peak lists into the algorithm. When samples were analyzed using reflector positive mode, the algorithm correctly identified 11 out of 12 (91.67%) unknown samples (see e.g., Table 2). In the one remaining sample, the correct species was assigned the highest probability, but this value was 0.02 less than the p-value adjusted threshold. Each prediction was assigned an overall high probability assignment with an average of 0.84±0.16 (standard deviation).

TABLE 2
Identification of unknown spiked blood samples

| Sample | Identification | Prediction Probability |
|---|---|---|
| Identity | RP Mode (m/z: 700-5K) | Average |
| C. albicans | + | 0.94 ± 0.01 |
| | + | |
| K. pneumoniae | + | 0.84 ± 0.17 |
| | + | |
| P. aeruginosa | + | 0.95 ± 0.06 |
| | + | |
| M. tuberculosis | + | 0.90 ± 0.11 |
| | + | |
| S. aureus | + | 0.66 ± 0.03 |
| | + | |
| E. coli | n/a | n/a |
| S. pyogenes | + | 1.00 ± 0.00 |
| | —* | |
| Totals: | 11/12 (91.6%) | 0.84 ± 0.16 |

In Table 2, + indicates correct identification; − indicates incorrect identification; +/− indicates more than one species met identification criteria, one of which is correct; and * indicates a non-significant prediction. Probability values include +/− predictions.

As shown in Table 2, two samples of each species were prepared from spiked blood as outlined in FIG. 2. An individual to whom the sample identity was unknown collected 5 spectra for each sample via MALDI-TOF MS in the desired m/z range. The species prediction whose assigned probability value lies above the p-value adjusted threshold (α=0.005) was selected as the identification output. Using reflector positive mode (m/z range: 700-5,000 Da) 11 out of 12 (91.6%) of unknown samples were correctly identified with a total average for positive identification being 0.84±0.16. The asterisk symbol (*) indicates that the second S. pyogenes sample was correctly identified, but this value was 0.02 less than the p-value adjusted threshold.

In order to determine the system sensitivity for sample identification, spiked blood samples were prepared at each ten-fold dilution of microbial lysate ranging from 1×10⁸ CFU/mL to 10 CFU/mL and tested for positive identification (see e.g., Table 3). Sensitivity was defined by the minimum concentration of pathogen lysate required for an accurate identification falling above the p-value adjusted threshold to be made. In order to more acutely evaluate the maximum sensitivity of the system, all samples were evaluated using reflector positive mode except for E. coli which was evaluated using linear positive mode. Samples were correctly identified as C. albicans, K pneumoniae, S. aureus, and E. coli for all sample concentrations as low as 10 CFU/mL equivalent of antibiotic-treated pathogen. The sensitivity varied with the remaining species with correct identification at 1×10⁴ CFU/mL of M. tuberculosis, 1×10⁷ CFU/mL of P. aeruginosa, and 1×10⁸ CFU/mL of S. pyogenes.

Overall, these results demonstrate the capacity of this system as a method of positive microbial identification directly from whole blood at clinically relevant infection levels.

TABLE 3
Sensitivity determination for the identification of
unknown spiked blood samples

| | Minimum | | |
|---|---|---|---|
| | concentration | | P-value |
| | (CFU/mL) of | | Adjusted |
| | antibiotic-treated | | Threshold |
| | pathogen | Probability | (α = 0.005) |
| C. albicans | 1 × 10¹ | 1.00 | 0.60 |
| K. pneumoniae | 1 × 10¹ | 0.89 | 0.38 |
| S. aureus | 1 × 10¹ | 0.45 | 0.44 |
| E. coli | 1 × 10¹ | 0.63 | 0.30 |
| M. tuberculosis | 1 × 10⁴ | 0.54 | 0.40 |
| S. pyogenes | 1 × 10⁸ | 0.88 | 0.37 |
| P. aeruginosa | 1 × 10⁷ | 0.61 | 0.31 |

As shown in Table 3, a titer was produced for each pathogen lysate by diluting down each culture ten-fold from 1×10⁸ to 1×10¹ CFU/mL equivalent of antibiotic-treated pathogen. Spiked blood samples were prepared at each concentration as outlined in FIG. 2. In order to determine the maximum sensitivity of the system, 5 spectra were collected for each species using reflector positive mode for all species except for E. coli samples which were evaluated under linear positive mode. The correct identification was consistently predicted for all samples as low as 1×10¹ CFU/mL for C. albicans, K. pneumoniae, S. aureus and E. coli spiked blood samples. A more limited sensitivity was observed with the remaining species with correct identification predicted down to 1×10⁴ CFU/mL for M. tuberculosis, 1×10⁷ CFU/mL for P. aeruginosa, and 1×10⁸ CFU/mL for S. pyogenes. Note that M. tuberculosis was purchased as inactivated via 2.4 mRads of gamma irradiation and bacterial concentrations were instead calculated by weight.

Assessment of Diagnostic Potential with Patient Blood Samples

To evaluate whether this system could be applied to accurately characterize infection in septic patients, clinical blood samples were collected from patients with suspected sepsis as a part of a secondary analysis to a multicenter study of early detection of inflammatory biomarkers in infection. Selected patients were clinically characterized as septic and were blood culture negative but had a positive secondary culture (e.g., wound, sputum, urine, or joint fluid) indicating infection with a confirmed or potential pathogen genus for which a fingerprint library had been developed. Of the 66 patient samples collected, 13 patients fit these criteria (see e.g., FIG. 1). The culture requirement was necessary to confirm the validity of the prediction output. Including multiple time points, a total of 18 blood samples were collected and analyzed. The baseline characteristics for these patients are outlined in Table 1. In order to more accurately recapitulate a clinical setting, the libraries of both reflector positive and linear positive mode were combined in order to simultaneously compare all 7 pathogen fingerprint libraries in one identification output while maintaining accuracy.

The diagnostic system correctly identified at least one blood sample of 7 out of 13 patients (53.84%) or 9 out of 18 (50%) of total blood samples at the genus level. At the group level, 10 out of 13 patients (76.92%) or 12 out of 18 (66.67%) blood samples were correctly identified (see e.g., Table 4). The culture information from 3 patients indicated only a Gram-positive cocci species. This could indicate infection with Staphylococcus or Streptococcus species and were thus included in the study, but the genus was not confirmed. An increase in inaccuracy was observed with blood samples containing multiple infections. Interestingly, all 4 patients who were co-infected with a species without an established pathogen library were incorrectly identified, indicating that the presence of non-specified peaks may have interfered with identification. If one were to select blood samples containing only a genus with a developed library, 6 out of 9 (66.67%) of samples were correctly identified at the genus level. Similarly, when selecting blood samples with only a singular infection, 4 out of 6 (66.67%) of total blood samples were correctly identified at the genus level. Taken together, this system can correctly identify clinical samples from blood culture negative septic patients, and it can be used as a clinical diagnostic tool.

TABLE 4
Clinical Sample Identification

| Patient | | | | Culture | | Genus | Group |
|---|---|---|---|---|---|---|---|
| ID | Time | Severity | PAMPs/mL | Site | Culture ID | Level | Level |
| 1079 | 0 | Sepsis | 90.65 ± 9.78 | Wound | S. aureus and | + | + |
| | | | | | Strep Group B | | |
| 1122 | 0 | Severe | 19.83 ± 0.31 | Sputum | Gram-positive | + | + |
| | | Sepsis | | | cocci | | |
| 1122 | 24 | Severe | 37.16 ± 1.13 | Sputum | Gram-positive | − | − |
| | | Sepsis | | | cocci | | |
| 1122 | 48 | Severe | 60.66 ± 6.82 | Sputum | Gram-positive | + | + |
| | | Sepsis | | | cocci | | |
| 1184 | 0 | Septic | 67.27 ± 2.21 | Urine | S. | − | + |
| | | Shock | | | saprophyticus | | |
| 1191 | 0 | Sepsis | 94.69 ± 5.46 | Wound | S. aureus, C. | * | * |
| | | | | | diptheroids | | |
| 1191 | 24 | Sepsis | 114.06 ± 3.57 | Wound | S. aureus, C. | − | + |
| | | | | | diptheroids | | |
| 3259 | 0 | Severe | 77.51 ± 4.37 | Wound | Positive cocci | + | + |
| | | Sepsis | | | | | |
| 3260 | 0 | Sepsis | 71.23 ± 2.11 | Sputum | Gram-positive | − | − |
| | | | | | cocci, Gram- | | |
| | | | | | positive rods | | |
| 3260 | 48 | Sepsis | 161.37 ± 18.23 | Sputum | Gram-positive | +/− | +/− |
| | | | | | cocci, Gram- | | |
| | | | | | positive rods | | |
| 3276 | 0 | Sepsis | 147.35 ± 0.00 | Urine | E. coli | + | + |
| 3276 | 24 | Sepsis | 129.84 ± 2.23 | Urine | E. coli | + | + |
| 5015 | 0 | Sepsis | 160.07 ± 11.08 | Urine | K. pneumoniae | + | + |
| 5059 | 0 | Severe | 63.39 ± 0.35 | Urine | P. aeruginosa, E. | − | n/a |
| | | Sepsis | | | faecalis | | |
| 5060 | 0 | Severe | 60.26 ± 0.70 | Urine | K. pneumoniae, A. | − | + |
| | | Sepsis | | | faecalis | | |
| 5064 | 0 | Severe | 230.7 ± 9.30 | Urine | E. coli, Enteric | + | + |
| | | Sepsis | | | Gram-negative | | |
| | | | | | rods | | |
| 5073 | 0 | Severe | 90.21 ± 0.00 | Urine | E. coli, K. | − | − |
| | | Sepsis | | | pneumoniae, P. | | |
| | | | | | aeruginosa | | |
| 7092 | 0 | Sepsis | 109.09 ± 8.37 | Joint Fluid | Strep Group G | − | − |

| | | |
|---|---|---|
| Total for Patients | 7/13 | 10/13 |
| | (53.84%) | (76.92%) |
| Total for Blood Samples | 9/18 | 12/18 |
| | (50.00%) | (66.67%) |

In Table 4, + indicates correct identification; − indicates incorrect identification; +/− indicates two species predictions were assigned equal probability, one of which was correct; and * indicates a non-significant prediction. In Table 4, group level is indicated as “n/a” if patient was infected with multiple bacteria of different groups.

As shown in Table 4, clinical blood samples were collected as a part of a secondary analysis to a multicenter study of early detection of inflammatory biomarkers in infection. This study included the collection of patient blood from four emergency departments. A total of 13 patients with suspected sepsis were included in the analysis. All patients were blood culture negative, but culture information from additional sites including wound, sputum, urine, or joint fluid indicated infection with a genus with a developed fingerprint library. A total of 18 blood samples were analyzed when including multiple time points. The algorithm correctly identified at least one blood sample from 7/13 (53.84%) patients or 9/18 (50.00%) of the total blood samples at the genus level and 10/13 (76.92%) patients and 12/18 (66.67%) of total blood samples at the group level.

As shown in Table 5, in order to investigate how treatment with different classes of antibiotics may affect the spectra of each species, a Gram-negative, P. aeruginosa, and a Gram-positive, S. pyogenes, were treated with 4 different classes of antibiotics. Bacterial cultures were grown to log phase and then treated with the antibiotic as indicated overnight. Bacterial death was confirmed by plating.

TABLE 5
Antibiotics administered to evaluate how differing mechanisms
of action may affect PAMPs spectra

| | | | | Bactericidal or | Mechanism |
|---|---|---|---|---|---|
| Species | Antibiotic Class | Antibiotic | Concentration | Bacteriostatic | of Action |
| S. pyogenes | Glycopeptide | Vancomycin | 0.5 mg/mL | Bactericidal | Inhibits |
| Gram- | | | | | peptidoglycan |
| positive | | | | | synthesis |
| | Cephalosporin | Cefepime | 1 mg/mL | Bactericidal | Disrupts |
| | | | | | peptidoglycan |
| | | | | | crosslinking |
| | Lipopeptide | Daptomycin | 1 mg/mL | Bactericidal | Depolarizes |
| | | | | | the bacterial |
| | | | | | membrane |
| | Lincosamide | Lincomycin | 1 mg/mL | Bacteriostatic | Binds to the |
| | | | | | 50S ribosomal |
| | | | | | subunit |
| | | | | | Inhibits |
| | | | | | translocation |
| P. | Aminoglycoside | Amikacin | 0.5 mg/mL | Bactericidal | Binds to the |
| aeruginosa | | | | | 30S ribosomal |
| Gram- | | | | | subunit |
| negative | | | | | Inhibits |
| | | | | | translocation |
| | Cephalosporin | Cefepime | 1 mg/mL | Bactericidal | Disrupts |
| | | | | | peptidoglycan |
| | | | | | crosslinking |
| | Carbapenem | Meropenem | 1 mg/mL | Bactericidal | Inhibits cell |
| | | | | | wall synthesis |
| | | | | | by binding to |
| | | | | | penicillin- |
| | | | | | binding |
| | | | | | protein (PBP) |
| | | | | | targets |
| | Quinolone | Ciprofloxacin | 1 mg/mL | Bacteriostatic | Binds to DNA |
| | | | | | gyrase and |
| | | | | | topoisomerase |
| | | | | | IV |
| | | | | | Inhibits cell |
| | | | | | division |

### Discussion

All currently available MALDI-TOF MS systems require a positive blood culture for the diagnosis of sepsis, which is time-consuming and only applicable to a small subset of patients (see e.g., Bacconi et al., 2014, supra; Christner et al., 2010, supra; Biswas and Rolain, 2013, supra; Cherkaoui et al., 2010, supra; Cartwright et al., 2016, supra; Ho and Reddy, 2010, supra; Idelevich et al, 2014, supra; Gille-Johnson et al., 2013, supra; Tsalik et al., 2012, supra; Didar et al., 2015, supra; van den Beld et al, 2019, supra). The current gold standard for patients suspected of sepsis includes immediate treatment with empirical broad-spectrum antibiotics, which may be insufficient or ineffective (see e.g., Bacconi et al., 2014, supra; Prost et al., 2013, supra). Without a positive culture, physicians are prevented from prescribing more targeted and efficacious therapies. There is a vital need for a diagnostic tool capable of identifying a pathogen directly from blood of infected patients within the framework of standard-of-care antibiotic use. Leveraging FcMBL superparamagnetic bead capture and MALDI-TOF MS analysis, described herein are methods of identification for antibiotic-treated pathogens requiring only 10 CFU/mL equivalent of pathogen lysate. This system can correctly identify infection in blood culture negative septic patients in only 90 minutes from blood draw. This system can be established as a clinical diagnostic tool to rapidly identify infection in blood culture negative septic patients.

In this study, method was developed to produce highly concentrated samples of PAMPs captured directly from whole blood using FcMBL-coated magnetic beads. A technique was developed to produce concentrated solutions of PAMPs directly from whole blood to be used for accurate pathogen identification via mass spectrometry and algorithmic analysis in only 90 minutes (see e.g., FIG. 2). This is a significant improvement from the 1-5 days required by current blood culture-dependent identification methods for the cultivation of live bacteria prior to analysis by mass spectrometry. Unique and consistent mass spectra were collected using two different m/z ranges for a diverse set of species from 7 clinically relevant genera: S. aureus, S. pyogenes, K. pneumoniae, P. aeruginosa, E. coli, C. albicans, and M. tuberculosis (see e.g., FIG. 4A-4B and FIG. 5A-5G). The Extended Prevalence of Infection in Intensive Care (EPIC II) study, which collected data from over 14,000 patients, identified S. aureus (20.5%), Streptococcus species (S. epidermidis (10.8%), S. pneumoniae (4.1%)) Klebsiella species (12.7%), Pseudomonas species (19.9%), and Candida species (17%) as the top most prevalent types of organisms in culture-positive infected patients (see e.g., Vincent et al. JAMA. 2009; 302: 2323-2329). M. tuberculosis was included within this study to demonstrate the ability to recognize mycobacteria in addition to other types of bacteria and fungi. Spectra were collected from multiple size ranges in order to include species whose sample content contained PAMPs of varying sizes, such as E. coli which could not be visualized in a smaller m/z range (see e.g., FIG. 4A-4B).

Marinach-Patrice et al. demonstrated that species identification with MALDI-TOF MS can be improved by using blood-spiked fingerprint libraries as the reference spectra in difficult-to-identify species (see e.g., Marinach-Patrice et al. 2010, PLoS One, 5(1), e8862). This was increasingly necessary in this system in order to identify a collection of PAMPs extracted out of whole blood. The algorithm was designed to evaluate each spectrum at a “granular” peak-clustered level, rather than evaluation of spectra similarity as a whole, managing to independently treat signal from noise in the spectra. Frequency weighting was performed to take care of cluster sizes, and the trustworthiness parameter was introduced to allow cluster composition to be accounted for when evaluating a sample identity. These parameters helped ignore the presence of noise-related, low frequency peaks in order to provide a more robust and accurate identification output. In a blinded study, the developed fingerprint libraries were applied as a reference to correctly identify 11 out of 12 (91.6%) unknown spiked blood samples when run in the smaller m/z range (see e.g., Table 4). The rate of successful identification seen with current MALDI-TOF MS systems is directly related to the amount of microorganism available on the plate. The ability of this system to extract and concentrate large quantities of PAMPs directly out of whole blood provides the advantage of identifying a pathogen at low levels of infection without the need for cultivation or enrichment. To more acutely evaluate the maximum sensitivity of the system identification capabilities, E. coli samples were evaluated with LP mode and the remaining species were evaluated using RP mode. The concentration of PAMPs with FcMBL-coated magnetic beads allowed for the correct identification of species at concentrations as low as 10 CFU/mL equivalent of antibiotic-treated pathogen for 4 out of the 7 species (see e.g., Table 3). A more limited sensitivity was observed with the remaining species with sensitivity to 1×10⁷ CFU/mL for P. aeruginosa and 1×10⁸ CFU/mL for S. pyogenes. However, there are limitations to testing sensitivity by spiking bacteria into blood from healthy donors, whose blood does not entirely represent the environment of septic patients. The decreased sensitivity could in part be related to uptake of PAMPs by red blood cells prior to FcMBL capture.

Blood samples were collected from 13 septic patients who were blood culture negative but who had a secondary culture indicating infection with a confirmed or potential genus for which a reference fingerprint library had been developed. The diagnostic system correctly identified at least one blood sample from 53.84% of patients or 50% of the total blood samples at the genus level and 76.92% of patients and 66.67% of total blood samples at the group level. Notably, 3 of the patients were culture positive for a Gram-positive cocci-shaped species which indicated infection with a Streptococcus or Staphylococcus species, but the specific genus was not confirmed. Additionally, an increase in accuracy to 66.67% total blood samples at the genus was observed when only selecting samples with a singular infection. Collectively, this system can identify infection in blood culture negative septic patients in only 90 minutes, and this technique can be applied as a rapid clinical diagnostic tool.

The system also accurately identified clinical samples in a proof-of-concept study. The accuracy and sensitivity of this assay is further increased by optimizing the quality of sample preparation and spectra generation, using isotopes to enhance differentiation between signal and noise peaks, and including mass corrections with internal standards in order to narrow the bin size during spectra analysis.

There are also several variations of the probabilistic algorithm applicable within a clinical setting. When using a limited number of developed pathogen fingerprint libraries as described herein, the algorithm implemented a strict method of peak elimination when analyzing unknowns, where clusters containing m/z peaks from more than two libraries are entirely eliminated. Variations of the algorithm require a less stringent approach to noise exclusion, especially with regards to expanded libraries. An expanded number of libraries and/or libraries with reduced noise levels also allow for an accurate “No Match” prediction of a species outside of the developed libraries or for multiple infections.

Overall, this method can be applied as a diagnostic tool to rapidly identify PAMPs directly from blood culture negative septic patients, which can be applied in the clinical laboratory. It is further contemplated that this system can also be used to determine antibiotic efficacy or bacterial resistance. As described herein, an expanded number of reference libraries allows for the identification of a greater number of organisms and the establishment of the scope and limitations of its use as a diagnostic. With an expanded number of libraries, variations of the algorithm used as a clinical diagnostic tool follow a less stringent approach to noise exclusion in order to predict a greater number of pathogens in a more robust manner.

### Example 2

A Variation of the Workflow that Increases Speed, Sensitivity, and the Number of Identified Microbes

Described herein is a variation of a MALDI-TOF sepsis diagnostic sample preparation procedure. The protocol below describes how to prepare and analyze samples with mass spectrometry via capture and concentration of PAMPs using FcMBL beads. New methods added to the procedure are indicated in steps 4, 5 and 7.

Step 1: Infected blood sample is incubated with FcMBL-coated beads for 30 minutes.

Step 2: Sample is washed and concentrated 16× in water using a magnetic tube rack for 5 minutes.

Step 3: Pathogen associated molecular particles are eluted from FcMBL-coated beads using heat for 30 minutes.

Step 4, the trypsin digest: 10% trypsin is added (2 uL trypsin to 20 uL sample), sample is placed in a 700 W microwave next to an open container of 100 mL of water and microwaved for 1 minute. The trypsin digest and microwave treatment of step 4 are new methods added to this procedure. 1 uL of sample is spotted on MALDI target plate.

Step 5: The target plate is evenly sprayed with matrix, which is a new method added to this procedure. For the spray, 2′,6′-Dihydroxyacetophenone in 50/50 MeOH/H₂O 0.1% formic acid is automatedly sprayed, which takes approximately 10 minutes using a HTX™-sprayer.

Step 6: The target plate is loaded on the MALDI mass spectrometer, and the mass spectra is obtained for the sample.

Sample masses are algorithmically matched to pathogenic mass libraries to determine infection identification. A new variation of the algorithm was used for analysis, wherein the range was extended to include smaller m/z values.

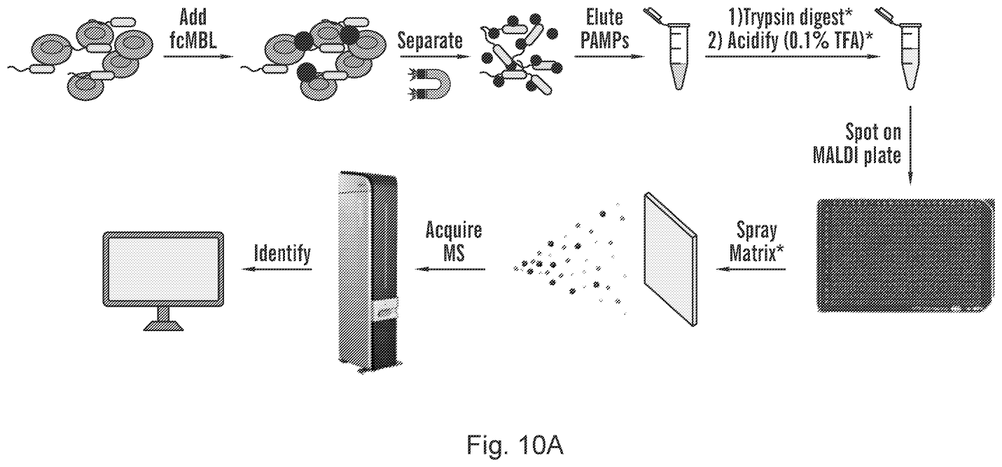

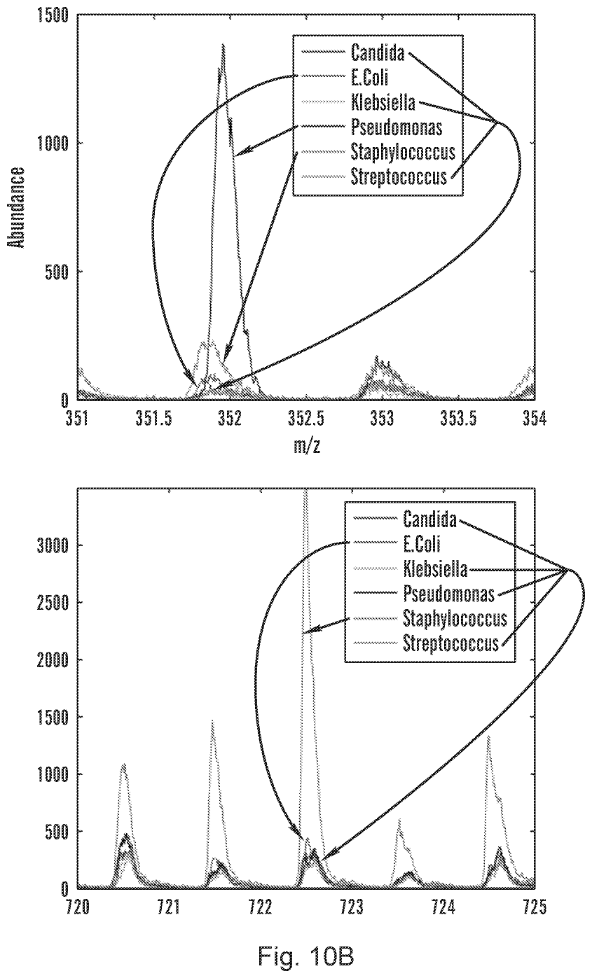

FIG. 10A is a schematic of the workflow variation, wherein an asterisk (*) indicates a newly integrated step that increased speed and sensitivity. FIG. 10B is a pair of graphs showing example MALDI-TOF spectra for C. albicans, E. coli, K. pneumoniae, P. aeruginosa, S. aureus, and S. pyogenes using the variation of the workflow described herein.

Improvements of this workflow variation compared to the method described in Example 1 include, but are not limited, to the following: (1) 100% (6/6) of the pathogens were correctly identified from whole blood samples; (2) matrix spray reduced sample preparation time 5-fold and increased throughput; (3) acidification and trypsin spray increased MS peak sensitivity and resolution; and (4) all analysis was performed under one mass spec method (reflector positive), and there was no need to acquire data multiple times for the same sample with different methods.

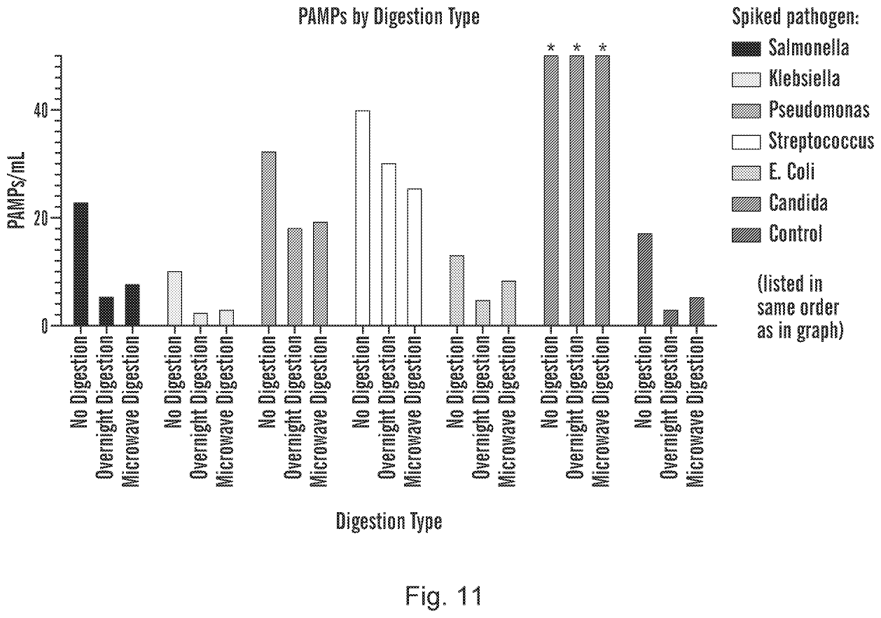

FIG. 11 is a bar graph comparing trypsin digestion methods. One-minute microwave trypsin digestion was compared to overnight trypsin digestion for S. aureus, K. pneumoniae, P. aeruginosa, S. pyogenes, C. albicans, E. coli, and a control. The concentration of PAMPs was quantified through ELLecSA. Note that 1 min microwave digestion was comparable to overnight digestion, and different microbes were digested at different efficiencies.

The identification rate was determined for 6 microbes (S. aureus, K. pneumoniae, P. aeruginosa, S. pyogenes, C. albicans, E. coli) using microwave trypsin digested samples or trypsin digested samples processed with a microwave trypsin library, a trypsin library, or a non-digested library; the non-digested library was obtained as described in Example 1. Note that both the trypsin and microwave trypsin libraries included 1-2 samples and 5-10 spots analyzed by MALDI-TOF MS. In some embodiments of any of the aspects, a trypsin library and/or a microwave trypsin library can include greater than 2 samples and greater than 10 spots analyzed by MALDI-TOF MS. For microwave trypsin digested samples, 6 of 6 pathogens were identified when processed with a microwave trypsin reference library, thus identifying at 100% probability. For non-digested samples, 1 of 6 pathogens were identified when processed with a non-digested reference library. For trypsin digested samples, 1 of 6 pathogens were identified when processed with a non-digested reference library. For trypsin digested samples, 5 of 6 pathogens were identified when processed with a trypsin reference library. For microwave trypsin digested samples, 2 of 6 pathogens were identified when processed with a trypsin reference library. These results are summarized in Table 6 below.

TABLE 6
Identification of microbes using different treatments
for sample and reference libraries

| | | | Percent |
|---|---|---|---|
| | | Pathogens | Pathogens |
| Sample Type | Reference Type | Identified | Identified |
| Microwave | Microwave | 6/6 | 100% |
| trypsin-digested | trypsin-digested | | |
| Non-digested | Non-digested | 1/6 | 16.67% |
| Trypsin-digested | Non-digested | 1/6 | 16.67% |
| Trypsin-digested | Trypsin-digested | 5/6 | 83.33% |
| Microwave | Trypsin-digested | 2/6 | 33.33% |
| trypsin-digested | | | |

### Example 3

Alternative Algorithm for MALDI-TOF Pathogen Identification

Described herein is an alternative method for identifying microbes using MALDI-TOF. Said method comprises a peak matching algorithm and comprises at least one of the following five features. (1) m/z values and peak areas are used to identify a pathogen. (2) bins m/z values in mass spectrum vary by 1 Da. m/z values vary slightly between runs, or within the same run, due to instrument drift Binning m/z peaks by 1 Da prevents the same peak from being treated as two different m/z values due to instrument drift. (3) The method removes m/z peaks that are associated with blood (control). (4) The method outputs the number of experimental replicates that contain a unique m/z value. This allows the assessment of repeatability between experimental replicates. (5) The method outputs an “Area Score” for peak areas that are significantly different between experimental and library datasets.

First datasets are acquired (see e.g., FIG. 12A). The workflow of the peak matching algorithm includes the following five steps: (1) upload, (2) filter, (3) pair m/z, (4) compare peak areas, and (5) output (see e.g., FIG. 12B).

Step 1: Datasets from the mass spectrometer (e.g., a Bruker™ machine) are uploaded. The input can comprise raw data (e.g., raw Bruker™ data) in an Excel™ spreadsheet. Data from each MALDI spectrum is located in a different tab. The output comprises an Excel™ spreadsheet with one tab, wherein all MALDI spectra are compiled under one tab (see e.g., FIG. 12B-12C). The m/z values are assigned to bins by 1 Da increments. A bin can be defined by the peak of highest intensity within the bin, not by the average m/z.

Step 2: The m/z values are then filtered (see e.g., FIG. 12B-12D). Step 2a: Five experimental replicates are run for each sample. This includes five experimental replicates for each library entry and for each experimental (i.e. unknown) sample. Not all experimental replicates for a given sample will contain the same m/z value (due to experimental variability). So, the fraction of experimental replicates from each sample that contain an m/z value to assess repeatability is determined. The “number of times that a matched m/z value appeared in the experimental replicate” can be called a “repeatability value”, and it is calculated by the following equation: “repeatability value”=(number of experimental replicates of a sample that contain a particular m/z value)/(number of experimental replicates). m/z values in the experimental dataset that have a “repeatability value” less than or equal to 0.4 are removed from the experimental dataset. m/z values in the control dataset that have a “repeatability value” less than or equal to 0.4 are removed from the control dataset. m/z values in the experimental dataset that match to the control dataset within +/−0.3 Da are removed from the experimental dataset. Output comprises m/z values unique to the experimental dataset (see e.g., FIG. 12B, 12D).

Step 2b: m/z values in the experimental dataset that do not match a m/z value in the library dataset within +/−0.3 Da are removed from the experimental dataset. Output comprises all m/z values in the experiment that match to at least one known microbe's library (see e.g., FIG. 12B, 12E).

Step 3: m/z values are combinatorially paired in a dataset. A list of all possible pairs of m/z values in the Library and Experimental datasets is generated. A peak area ratio is then calculated for each pair. FIG. 12F shows an exemplary experimental dataset, which was sorted against the Klebsiella library in Step 2b, and then all possible combinations of two peaks “A” and “B” is generated within the experimental dataset. A peak area ratio is then calculated between each A and B pair. Formula 4 below shows the calculation for the peak area ratio (%), wherein “AreaA” is the area of a first peak from a dataset and “AreaB” is the area of a second peak from the same dataset as AreaA. Step 3 thus outputs all possible m/z pairs in the experimental dataset and all possible m/z pairs in a microbe library and calculates the peak area ratio for each pair. Step 3 can be repeated for each microbe library and an experimental dataset sorted against that microbe library (see e.g., FIG. 12B, 12F).

$$= \frac{{Area}_{B}}{{Area}_{B} + {Area}_{A}} \times 100 \qquad ( 4 )$$

Step 4: The peak area ratios are then compared for each matching peak area ratio (i.e., same m/z pair) between the experimental dataset and microbe library. The percent difference between each matching peak area ratio is calculated. FIG. 12G shows an exemplary comparison of matching peak area ratios between the experimental dataset and a sorted Klebsiella library, where each row corresponds to a unique combination of m/z A and m/z B. If the percent difference between the matching peak area ratios is at or below a specific threshold (e.g., 15% or less), then a conclusion is output as true. If the percent difference between the matching peak area ratios is greater than the specific threshold (e.g., 15%), then the conclusion is output as false. If the conclusion is output as false, then the difference between the matching peak area ratios is output and designated as the “Area Score” (also referred to herein as a “Difference Score” or a “Peak Area Difference Score”). If the conclusion is output as true, then the Area Score is zero (0). Note that not all peak area ratios calculated are shown in FIG. 12G, just a representative selection (see e.g., FIG. 12B, 12G).

Step 5: An output table is generated that compares the experimental sample to all pathogens in the library. The output table of the peak matching algorithm comprises at least one of the following three features: (i) a “difference score”, corresponding to the difference between the peak area ratios in the library and experimental datasets if the ratio difference exceeded a cutoff or threshold (e.g., 15%); (ii) number of m/z values in the experimental sample that matched to a microbe's library (e.g., within +/−0.3 Da); and (iii) the percent of area pairs in the experimental dataset that matched to a specific microbe's library. The “Peak Area Difference Score” in FIG. 12H is the sum of all “Area Scores” from the “Area Scores” column in FIG. 12G (Step 4). The sum of all “Area Scores” generates one “Peak Area Difference Score” for each microbe. The “percent of area pairs in the experiment that matched to this microbe's library” refers to the percent of Peak Area Ratios that match between an experimental sample and a microbe in the library. If the Peak Area Ratio of AreaA and AreaB in the experimental dataset lies within 15% of the Peak Area Ratio of the same AreaA and AreaB in the library dataset, it is considered a match and the number of matches is used to calculate the percent value. The output table contains n rows, corresponding to n number of pathogens in the library (see e.g., FIG. 12B, 12H).

The microbe in the experimental sample is identified as belonging to a specific microbe's library based on minimizing the value of (i) above, and maximizing the value of (ii) and (iii) above. As a non-limiting example in FIG. 12G, the microbe in the experimental dataset is identified as belonging to Candida because: (i) the peak area difference score is the minimum value in the output table (e.g., 0); (ii) the number of m/z values in the experimental dataset that matched to the Candida library was the maximum value in the output table (e.g., 10); and (iii) the percent of area pairs in the experimental dataset that matched to the Candida library was the maximum value in the output table (e.g., 1 or 100%).

### Example 4

In some embodiments of any the aspects, the methods described herein use FcMBL coated, non-magnetic beads, e.g., based on mesoporous silica (MPS). In some embodiments of any of the aspects, the mesoporous silica comprises a slurry of silica rods that forms a scaffold material. The advantage is that the total FcMBL-MPS bead with captured PAMPs can be mixed with the matrix and introduced directly into the MALDI-MS. This avoids the step of elution from the magnetic bead at 70° C.; the elution step is necessary to avoid contaminating the MALDI machine with the magnetic particles.

### EDC Coupling of FcMBL to Carboxyl Functionalized Mesoporous Silica

Purpose: This is a protocol for the covalent coupling of FcMBL protein to carboxyl functionalized mesoporous silica (MPS).

NOTE: Prepare or buy all buffers in low endotoxin water (if possible). FcMBL binds endotoxin

Equipment: ThermoFisher™ HulaMixer™ sample mixer (Thermo Fisher™ 15920D); Magnetic rack for 50 ml tube; 14×KJ Magnetics™ BY044+3D printed holder; 50 ml conical tubes (VWR 21008-178); 10 ml Serological pipettes (Becton Dickinson™ 356551); Pipettors including Pipet-Lite™ LTS L-1000 (Rainin™ 17014382), Pipet-Lite™ LTS L-200 (Rainin™ 17014391), Pipet-Lite™ LTS L-20 (Rainin™ 17014392), Pipet-Lite™ LTS L-10 (Rainin™ 17014388); Pipette tips including 1000 ul filter tips (Rainin™ RT-L1000F), 200 ul filter tips (Rainin™ RT-L200F), 20 ul filter tips (Rainin™ RT-L10F); Lab vortex (Scientific Industries™ SI-0236); Rocking lab shaker (Benchmark Benchrocker™ BR2000); Probe sonicator (Qsonica™ Q700-110 with ⅛″ microtip).

Materials (as listed or equivalent): FcMBL protein (a specific mg/ml stock, as indicated on tube); Carboxylated MPS (e.g., prepared in house); EDC (1-Ethyl-3-(3 dimethylaminopropyl) carbodiimide, ThermoFisher™ 22980); NHS (N-hydroxysuccinimide, ThermoFisher™ 24500); 25 mM MES pH 5 (dilute MES buffer pH 5.0 (Boston BioProducts™ BB-99) to 25 mM); 1 mg/ml BSA (item 2.3.10) in 25 mM MES pH 5 (item 2.3.5); PBST buffer (add 0.05% TWEEN-20 to DPBS); Glycerol (Sigma™ 65516); BSA (Bovine Serum Albumin, Sigma™ A3803); 1M Glycine solution (dissolve 3.75 g glycine (Amresco™ 0167) in 50 ml water); DPBS buffer (Alfa Aesar™ J61917); 0.5M EDTA (Thermo™ 15575020).

Procedures:

Weigh out and resuspend Carboxylated MPS to 5 mg/ml in 25 mM MES buffer.

Perform wash: Place tube in rack and allow to settle for 2-3 minutes. All beads should be out of solution. Carefully pipette off supernatant. Add original buffer volume and vortex to resuspend. Repeat wash once more with original volume of 25 mM MES. Resuspend beads at 16.67 mg/ml in 25 mM MES (should be 30% of original volume).

Dissolve EDC at 50 mg/ml in 25 mM MES buffer. This must be done immediately before use. If EDC was stored in freezer, allow to warm to room temperature before opening to prevent condensation inside bottle. Moisture will degrade EDC.

Dissolve NHS at 50 mg/ml in 25 mM MES buffer. Add NHS, 12.5% of total volume, and EDC, 12.5% of total volume, to beads in tube (total volume should be 40% of original volume) (i.e., 800 ul for original volume of 2 ml, of which 100 ul would be NHS and 100 ul would be EDC). Mix for 30 min on hula mixer at 10 rpm. Perform 2× washes, as described above, with 25 mM MES.

Resuspend beads in 50% original volume: add 25 mM MES buffer to final volume. For 1 ml: 1000 ul-400 ul of 1 mg/ml BSA−100 ul of 1 mg/ml FcMBL=500 ul 25 mM MES. Add BSA to make 400 μg/mL BSA. Add FcMBL to make 100 μg/mL FcMBL. Mix for 30 min on hula mixer at 10 rpm. Perform 3× washes, as described above, with original volume PBST. Add Glycine to the beads in PBST to 30 mM. Mix for 30 min on hula mixer at 10 rpm. Perform wash with original volume of PBS and resuspend in original volume of PBS with EDTA to 10 mM. Store at 4 C.

### MPS Bead FcMBL Conjugation Protocol

Weigh dry carboxylated-MPS beads and dispense into conical tubes. Resuspend the dry powder in 25 mM MES pH 5.0 buffer (i.e., 2-(N-morpholino)ethanesulfonic acid) to a concentration of 5.0 mg/mL. Wash the MPS beads in MES, place the tubes in a rack, and allow the beads to settle for several minutes. Aspirate the supernatant with a pipette and resuspend the beads in MES buffer. Repeat the wash one more time. After the second wash, resuspend the MPS beads in MES to 30% of the original volume or 16.67 mg/mL.

Prior to FcMBL conjugation, prepare 50 mg/mL solutions of 41-ethyl-3-(3-dimethylamino) propyl carbodiimide (EDC) and N-hydroxysuccinimide (NHS) in 25 mM MES pH 5.0. Add NHS, 12.5% of total volume, and EDC, 12.5% of total volume, to beads in tube (total volume should be 40% of original volume) (e.g., 800 ul for original volume of 2 ml, of which 100 ul would be NHS and 100 ul would be EDC). Mix for 30 minutes on a hula shaker. Perform two washes as in the previous step.

Resuspend the MPS beads to 50% of the original pre-wash volume in 25 mM MES with Bovine Serum Albumin (BSA) and FcMBL. The new MPS bead concentration is 10 mg/mL. Half of this resuspended volume is 25 mM MES pH 5.0. Four-tenths of the volume is a 1 mg/mL solution of BSA in 25 mM MES pH 5.0. One-tenth of the volume is a 1 mg/mL solution of FcMBL. Mix for 30 minutes on a Hula shaker at 10 RPM.

After mixing, perform three washes with PBS-T (phosphate buffered saline with detergent such as TWEEN 20 or TRITON X-100). Allow the MPS beads to settle and replace the supernatant. Return the beads to the original concentration (5 mg/mL). Add 1M Glycine to the beads in PBS-T, so that the mixture is 30 mM in Glycine. Mix on the Hula shaker for 10 minutes at 10 rotations per minute (RPM).

Perform washes with PBS at the initial starting volume. To complete the washes, resuspend the MPS beads in PBS with 10 mM ethylenediaminetetraacetic acid (EDTA). The final bead concentration should be 5 mg/mL. Store the beads at 4° C.

The MPS-FcMBL beads can be used to detect microbes or microbe components using a mass spectrometric method as described further herein (see e.g., FIG. 16).

What is claimed herein is:

1. A method of detecting a microbe or microbe component, the method comprising the following steps:
i) contacting a sample with an engineered microbe-targeting molecule linked to a support;
ii) isolating the microbe or microbe components bound to the engineered microbe-targeting molecule;
iii) contacting the microbe or microbe components with a matrix or matrix solution on a target substrate; and
iv) detecting the microbe or microbe components using a mass spectrometric method.

2. A method of detecting a microbial infection in a patient, the method comprising the following steps:
i) contacting a patient sample with an engineered microbe-targeting molecule linked to a support;
ii) isolating the microbe or microbe components bound to the engineered microbe-targeting molecule;
iii) contacting the microbe or microbe components with a matrix or matrix solution on a target substrate; and
iv) detecting the microbe or microbe components using a mass spectrometric method.

3. The method of any of claims 1-2, wherein the microbe components comprise microbe-associated molecular patterns (MAMPs).

4. The method of any of claims 1-3, wherein the microbe components comprise pathogen-associated molecular patterns (PAMPs).

5. The method of any of claims 1-4, wherein the detecting of step iv outputs mass spectrometric data obtained from the sample as a sample library.

6. The method of any of claims 1-5, wherein the detecting of step iv comprises comparing the sample library with at least one control library of mass spectrometric data.

7. The method of claim 6, wherein the at least one control library of mass spectrometric data comprises data obtained from at least one control sample not comprising any known microbes or microbe components.

8. The method of any of claims 1-7, wherein the detecting of step iv comprises comparing the sample library with at least one reference library of mass spectrometric data.

9. The method of claim 8, wherein the at least one reference library of mass spectrometric data comprises data obtained from at least one sample comprising a known microbe or components of at least one known microbe.

10. The method of any of claims 1-9, wherein the detecting of step iv comprises analyzing the sample library with a control system comprising one or more processors, the control system configured to execute machine executable code using a clustering process, wherein each cluster comprises a cluster of data points from a single molecular signal of interest.

11. The method of claim 10, wherein each cluster of data points is at least 1 Dalton (Da) wide.

12. The method of any of claims 10-11, wherein the cluster of data points is based on m/z peaks identified by the maximum intensity of that cluster.

13. The method of any of claims 10-11, wherein the cluster of data points is based on m/z peaks identified by the mean m/z value of that cluster.

14. The method of any of claims 5-13, wherein the detection process further comprises removing at least one data point of the sample library or the control library, wherein the at least one data point comprises a repeatability value at or below a pre-determined threshold.

15. The method of any of claims 10-14, wherein the clustering process further comprises removing at least one data point of the sample library that matches a data point in a control library within +/−0.3 Da.

16. The method of any of claims 10-15, wherein the clustering process further comprises removing at least one data point of the sample library that does not match a data point in at least one reference library within +/−0.3 Da.

17. The method of any of claims 1-16, wherein the detecting of step iv comprises determining a peak area ratio of at least one pair of data points within the sample library and at least one pair of data points in at least one reference library.

18. The method of claim 17, wherein at least one peak area ratio of the sample library is compared to at least one corresponding peak area ratio of at least one reference library.

19. The method of claim 18, wherein a score is calculated based on the comparison of at least one peak area ratio of the sample library and at least one corresponding peak area ratio of at least one reference library.

20. The method of any of claims 10-19, wherein the clustering process further comprises applying a weighting parameter, comprising a frequency weighting parameter and/or a trustworthiness weighting parameter, wherein the weighting parameter identifies the proportion of data points that are unique to the sample library or common with a reference library or a control library.

21. The method of claim 20, wherein the frequency weighting parameter increases the weight of a data point if the cluster containing the data point comprises additional other data points.

22. The method of claim 20, wherein the trustworthiness weighting parameter decreases the weight of a data point if the data point is found within multiple clusters in the sample library, reference library, and/or control library.

23. The method of any of claims 1-22, further comprising:
i) assigning a score to the sample based on similarity with a reference library; and/or
ii) identifying the microbe in the sample as belonging to a reference library based on the score being above a predetermined threshold.

24. The method of any of claims 1-23, further comprising identifying the species of the microbe detected in the sample according to the data points analyzed and outputting said species on a display.

25. The method of any of claims 1-24, further comprising identifying the strain of the microbe detected in the sample according to the data points analyzed and outputting said strain on a display.

26. The method of any of claims 1-25, further comprising determining whether the microbe detected in the sample is sensitive or resistant to an antimicrobial therapeutic according to the data points analyzed and outputting said sensitivity on a display.

27. The method of any of claims 1-26, further comprising assigning the patient to an infection category according to the data points analyzed and outputting the infection category on a display.

28. The method of any of claims 1-27, wherein the results of step iv comprise a profile, wherein said profile indicates the presence or absence of at least one microbe or microbe component.

29. The method of claim 28, wherein the profile is specific to at least one microbe or microbe component.

30. The method of any of claims 28-29, wherein the profile comprises a set of data points for a specific microbe or specific set of microbes, and wherein each profile comprises a set of m/z peaks clustered for a single molecular signal of interest.

31. The method of any of claims 28-30, wherein the profile for the specific microbe does not include any of the set of data points associated with a control library.

32. The method of any of claims 28-31, wherein the profile is distinguishable from the profiles of other microbes or microbe components or sets thereof.

33. The method of any of claims 28-32, wherein the profile is set forth in any one of FIG. 4A-4B, FIG. 5A-5G, FIG. 8B-8J, or FIG. 9A-9B.

34. The method of any of claims 1-33, wherein the support is a magnetic support.

35. The method of any of claims 1-34, wherein the support is a non-magnetic support.

36. The method of any of claims 1-35, wherein the support is a non-magnetic nanoparticle.

37. The method of any of claims 1-36, wherein the support is a mesoporous nanoparticle.

38. The method of any of claims 1-37, wherein the support is mesoporous silica.

39. The method of any of claims 1-38, wherein the step of isolating comprises applying a magnet to the sample.

40. The method of any of claims 1-39, wherein the step of isolating comprises washing the support with a buffer to remove unbound cells or biomolecules.

41. The method of any of claims 1-40, wherein the step of isolating further comprises eluting the microbe or microbe components from the support.

42. The method of claim 41, wherein the step of eluting comprises heating to a temperature of at least 70° C. and/or shaking at a speed of at least 950 rpm for no longer than 30 minutes.

43. The method of claim 42, wherein the heating to a temperature of at least 70° C. is performed in calcium-free water.

44. The method of claim 43, wherein the step of eluting comprises treatment with ethylenediaminetetraacetic acid (EDTA).

45. The method of any of claims 1-44, wherein the step of isolating does not comprise eluting the microbe or microbe components from the support.

46. The method of any of claims 1-45, wherein the step of isolating comprises concentrating the microbe or microbe components into a smaller volume from a larger volume of the sample.

47. The method of claim 46, wherein the isolated volume is less than the volume of the sample.

48. The method of any of claims 1-47, wherein the target substrate is evenly sprayed with matrix solution prior to step iii to generate a homogenous layer of crystallized matrix on top of the target substrate.

49. The method of any of claims 1-48, wherein the matrix solution comprises a matrix selected from the group consisting of 2′,6′-dihydroxyacetophenone (DHAP), α-Cyano-4-hydroxycinnamic acid (CHCA), sinapic acid (SA), super DHB, 2′,4′,6′-trihydroxyacetophenone monohydrate (THAP), and 9-aminoacridine (9-AA), and the matrix is dissolved in an organic, aqueous solution.

50. The method of any of claims 1-49, wherein the matrix solution is 40 mg/mL 2,5-Dihydroxybenzoic acid (DHB) in 50% methanol, 50% water, 0.1% formic acid.

51. The method of any of claims 1-50, wherein the mass spectrometric method is Matrix-Assisted Laser Desorption Ionization (MALDI-TOF).

52. The method of any of claims 1-51, wherein the mass spectrometric method is automated.

53. The method of any of claims 1-52, wherein the sample comprises blood, serum, plasma, sputum, urine, joint fluid, or any other tissue or biological sample.

54. The method of any of claims 2-53, wherein the patient has been treated with antibiotics.

55. The method of any of claims 1-54, wherein the sample contains at least one antibiotic.

56. The method of any of claims 1-55, wherein the sample contains at least two antibiotics.

57. The method of any of claims 2-56, wherein the patient has been treated with antifungals.

58. The method of any of claims 1-57, wherein the sample contains antifungals.

59. The method of any of claims 2-58, wherein the patient has been treated with antivirals.

60. The method of any of claims 1-59, wherein the sample contains antivirals.

61. The method of any of claims 1-60, wherein the sample has not been cultured.

62. The method of any of claims 1-61, wherein the time from the step of collecting the sample to the end of detecting takes equal to or less than 90 minutes.

63. The method of any of claims 1-62, wherein the engineered microbe-targeting molecule comprises a microbe surface-binding domain.

64. The method of any of claims 1-63, wherein the microbe surface-binding domain comprises a mannose-binding lectin (MBL).

65. The method of any of claims 1-64, wherein the microbe surface-binding domain comprises a human mannose-binding lectin (MBL).

66. The method of any of claims 1-65, wherein the microbe surface-binding domain comprises a carbohydrate recognition domain (CRD) of MBL.

67. The method of claim 66, wherein the CRD is linked to an immunoglobulin or fragment thereof.

68. The method of any of claims 66-67, wherein the CRD is linked to an Fc component of human IgG1 (FcMBL).

69. The method of claim 68, wherein the magnetic support is a superparamagnetic support.

70. The method of claim 68, wherein the magnetic support comprises a magnetic bead, a superparamagnetic bead, or a magnetic microbead.

71. The method of any of claims 1-70, wherein the engineered microbe-targeting molecule comprises FcMBL streptavidin linked to superparamagnetic beads.

72. The method of any of claims 1-71, wherein the engineered microbe-targeting molecule comprises FcMBL linked to mesoporous silica particles.

73. The method of any of claims 1-72, wherein the engineered microbe-targeting molecule is linked to an ELISA plate.

74. The method of any of claims 1-73, wherein the microbe comprises a Gram-positive bacterial species, a Gram-negative bacterial species, a mycobacterium, a fungus, a parasite, or a virus.

75. The method of any of claims 1-74, wherein the microbial component comprises a component from a Gram-positive bacterial species, a Gram-negative bacterial species, a mycobacterium, a fungus, a parasite, or a virus.

76. The method of claim 75, wherein the virus is a coronavirus.

77. The method of claim 75, wherein the virus is a severe acute respiratory syndrome coronavirus 2 (SARS-CoV-2).

78. The method of claim 75, wherein the Gram-positive bacterial species comprises bacteria from the class Bacilli.

79. The method of claim 75, wherein the Gram-negative bacterial species comprises bacteria from the class Gammaproteobacteria.

80. The method of claim 75, wherein the mycobacterium comprises bacteria from the class Actinobacteria.

81. The method of claim 75, wherein the fungus comprises fungus from the class Saccharomycetes.

82. The method of any of claims 1-81, wherein the microbe is selected from the group consisting of Staphylococcus aureus, Streptococcus pyogenes, Klebsiella pneumoniae, Pseudomonas aeruginosa, Mycobacterium tuberculosis, Candida albicans, or Escherichia coli.

83. The method of any of claims 1-82, wherein the microbe is a human pathogen.

84. The method of any of claims 1-83, wherein the sample contains at least one pathogen.

85. The method of any of claims 1-84, wherein the sample contains more than one pathogen.

86. The method of any of claims 1-85, wherein the species of the pathogen is identified.

87. The method of any of claims 1-86, wherein the strain of the pathogen is identified.

88. The method of any of claims 1-87, wherein the drug sensitivity of the pathogen is identified.

89. The method of any of claims 1-88, further comprising providing a therapy model to the patient based on the infection category assigned to the patient.

90. The method of any of claims 1-89, further comprising providing a therapy model to the patient based on the identified pathogen assigned to the patient.

91. The method of any of claims 1-90, wherein the therapy model comprises treatment with a therapeutic agent specific to the pathogen.


SEQUENCE LISTING (17 sequences)

SEQ ID NO: 1 - protein, 248 aa, Homo sapiens
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 2 - protein, 228 aa, Homo sapiens
ETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 3 - protein, 141 aa, Homo sapiens
AASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 4 - protein, 115 aa, Homo sapiens
VGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 5 - protein, 148 aa, Homo sapiens
PDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 6 - protein, 380 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 7 - protein, 383 aa, Artificial Sequence
  synthetic polypeptide
AKTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 8 - protein, 351 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATSKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 9 - protein, 232 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 10 - protein, 2 aa, Homo sapiens
ND

SEQ ID NO: 11 - protein, 3 aa, Homo sapiens
  misc_feature (2)..(2): Xaa can be any naturally occurring amino acid
EXN

SEQ ID NO: 12 - protein, 10 aa, Homo sapiens
NEGEPNNAGS

SEQ ID NO: 13 - protein, 9 aa, Homo sapiens
GSDEDCVLL

SEQ ID NO: 14 - protein, 15 aa, Homo sapiens
LLLKNGQWNDVPCST

SEQ ID NO: 15 - protein, 247 aa, Homo sapiens
SLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 16 - protein, 381 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAPDGDSSLAASERKALQTEMARIKKWLTFSLGKQVGNKFFLTNGEINITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 17 - protein, 352 aa, Artificial Sequence
  synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGATSKQVGNKFFLTNGEEVITFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI